(19) United States
(12) Patent Application Publication
(10) Pub. No.: US 2011/0039893 A1
(43) Pub. Date: Feb. 17, 2011

(54) GSK-3BETA INHIBITOR

(75) Inventors: Masakuni Kori, Osaka (JP); Hideyuki Oki, Osaka (JP); Tetsuya Tsukamoto, Osaka (JP); Masashi Takahashi, Osaka (JP); Masaki Setoh, Osaka (JP); Takehiro Hirano, Osaka (JP)

Correspondence Address: WENDEROTH, LIND & PONACK, L.L.P., 1030 15th Street, N.W.,, Suite 400 East, Washington, DC, 20005-1503, US

(73) Assignee: Takeda Pharmaceutical Company Limited

(21) Appl. No.: 12/311,653

(22) PCT Filed: Oct. 10, 2007
(86) PCT No.: PCT/JP2007/069738

§ 371 Date: Apr. 8, 2009

(30) Foreign Application Priority Data
Oct. 11, 2006 (JP) 2006 278026

(51) Int. Cl. A61K 31/444; C07D 401/14; A61P 25/00; A61P 3/10

(52) U.S. Cl. 514/333; 546/256

(57) ABSTRACT

For the purpose of providing a GSK-3β inhibitor containing a 2-aminopyridine compound or a salt thereof or a prodrug thereof useful as an agent for the prophylaxis or treatment of a GSK-3β-related pathology or disease, the present invention provides a GSK-3β inhibitor containing a compound represented by the formula (IA):

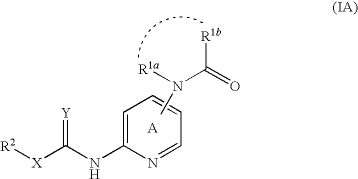

wherein each symbol is as defined in the specification.
or a salt thereof or a prodrug thereof.

### TECHNICAL FIELD

The present invention relates to 2-aminopyridine compounds having a Glycogen Synthase Kinase 3 (GSK-3) inhibitory activity, which are useful as pharmaceutical agents, and use thereof.

### BACKGROUND ART

GSK-3 was found to be a kinase that phosphorylates and deactivates glycogen synthase. It has been clarified at present that it is involved in the oxidation and synthesis of fatty acid, or abnormality in insulin signaling pathway via phosphates of various protein groups related to metabolism and signal transduction such as AcylCoA carboxylase, ATP-citrate lyase, Insulin receptor substrate-1 and the like. Moreover, GSK-3 is known to phosphorylate various structural proteins and regulate functions thereof. Particularly, phosphorylation of tau protein has been attracting attention in relation to the onset of Alzheimer's disease. In addition, GSK-3 is involved in phosphorylation of various transcription factors, and particularly, activates activator protein-1, cyclic AMP response element binding protein, nuclear factor of activated T cells, heat shock factor-1, b-catenin, Myc, C/EBP, NFκ-b or the like. Therefore, its inhibitor is expected to be a therapeutic drug for Alzheimer's disease, cerebral stroke, bipolar disorder, schizophrenia, cancer, bone disease, type II diabetes and obesity.

In insulin signaling pathway, GSK-3 is negatively regulated by phosphorylation via Akt (protein kinase B: also described as PKB). In diabetic patients, increased activity of GSK-3 and synthesis of fatty acids and/or insulin resistance are considered to be synergistically induced by the overlapped occurrence of promoted GSK-3 gene expression and insulin dysfunction. Since GSK-3 positively regulates the process of adipocyte differentiation and/or maturation via phosphorylation of C/EBP, increased GSK-3 activity triggers obesity, which in turn aggravates diabetes. In fact, it has been reported that administration of GSK-3 inhibitor improves insulin resistance of model animals of Type II diabetes. We have elucidated as our own findings that GSK-3 inhibitor suppresses adipocyte differentiation and/or maturation, expresses an antiobesity effect, and promotes sugar-dependent insulin secretory action of pancreatic P cells. Given these findings in combination, GSK-3 is considered to be additively and/or synergistically involved in the onset of diabetes in the insulin targeting tissues such as liver, skeletal muscle, fat, pancreas and the like, and GSK-3 inhibitor can be an effective therapeutic drug for obesity and/or diabetes because it eliminates these factors.

Activation of GSK-3 in Alzheimer's brain has been reported, and therefore, GSK-3 is considered to be involved in senile plaque and neurofibrillary tangle, which are the two major pathological findings in Alzheimer's disease. In the metabolism of amyloid precursor proteins, GSK-3 is linked to γ secretase to positively regulate the production of β amyloid protein, a main constituent component of senile plaque. As for tau protein, which is a main constituent component of neurofibrillary tangle, GSK-3 is considered to facilitate phosphorylation of the protein, prevent axonal transport, and finally induce neurodegeneration. It is also known that GSK-3 is located downstream of the PI3 kinase—Akt system signal transduction important for the neuronal cell survival, and activated during neuronal cell death. Accordingly, GSK-3 inhibitor is expected to not only suppress neurodegeneration but also suppress two major pathological findings of Alzheimer's disease. As our own findings, we have clarified that PI3 kinase—Akt system signal transduction plays a key role in neurogenesis and neuroregeneration and found that inhibition of GSK-3 located downstream thereof can facilitate neurogenesis. Considering our new findings in combination, there is a possibility that GSK-3 inhibitor suppresses two major pathological findings of Alzheimer's disease and additionally suppresses neurodegeneration, induces neurogenesis and achieves regeneration of function. It is assumed that GSK-3 inhibitor having the above-mentioned properties can be an ultimate therapeutic drug for Alzheimer's disease, and can also be effective as a therapeutic drug for neurodegenerative diseases such as Parkinson's disease and the like, cerebrovascular disorders and the like. Since a report has recently documented that Akt system signal transduction decreases in schizophrenia, GSK-3 inhibitor may become a completely new type of therapeutic drug for schizophrenia.

The following are known as regards the relationship between GSK-3 and diseases such as neurological disorder, diabetes, cancer, inflammatory disease (sepsis shock etc.), osteoporosis, alopecia and the like.

In neurological disorder, GSK-3 relates to neuronal cell death and nerve cell survival, and induces apoptosis by overexpression of GSK-3 (J. Biol. Chem. 273, 19929-19932 (1998)). In addition, GSK-3 phosphorylates tau protein which causes neurofibrillary tangle (Acta Neuropathology, 103, 91 (2002)).

In diabetes, GSK-3 phosphorylates glycogen synthase to decrease the activity, and inhibits glucose uptake in skeletal muscle to decrease the insulin reactivity (Diabetes 49, 263-271 (2000) and Diabetes 50, 937-946 (2001)).

In cancer, a GSK-3 inhibitor induced apoptosis in a certain kind of cancer cells (Mol. Cancer Ther. 2, 1215-1222 (2003)).

In inflammatory disease (sepsis shock etc.), GSK-3 acts on Toll-like receptor signal, and controls the production of inflammatory and anti-inflammatory cytokines. Since inhibition of GSK-3 suppresses production of inflammatory cytokine and increases production of anti-inflammatory cytokine, a GSK-3 inhibitor is useful for inflammatory diseases (Nature Immunology, 6, 777-784 (2005)).

In osteoporosis and alopecia, GSK-3 stabilizes β-catenin via Wnt signal, and is involved in bone mass increase and hair development (Journal of Bone Mineral Research, 21, 910-920 (2006) and Cell, 95, 605 (1998)).

As a compound having GSK-3β inhibitory activity, a compound represented by formula:

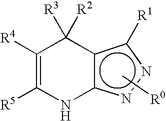

wherein
R⁰ is hydrogen, alkyl or the like;
R¹ is hydrogen;
R² is hydrogen, alkyl or the like;
R³ is (1) alkyl or haloalkyl, (2) cycloalkyl optionally having substituent(s), or the like;
R⁴ is alkoxycarbonyl, alkylcarbonyl or the like; and
R⁵ is alkyl, phenylaminoalkyl or the like, is known (WO04/014910).

Bioorg. Med. Chem. Lett. (2002), 12, 1525-1528 describes a compound having GSK-3β inhibitory activity which is represented by the following formula:

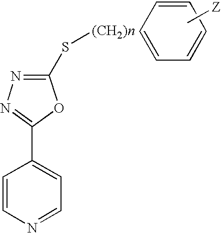

wherein
(1) n=1, Z═H; (2) n=2, Z═H; (3) n=1, Z=3-Cl; (4) n=1, Z=2-Cl; (5) n=1, Z=3-I; (6) n=1, Z=4-I; (7) n=1, Z=3-F; (8) n=1, Z=3-COOH; (9) n=1, Z=3-COOCH₃; (10) n=1, Z=3-(5-methyl-1,3,4-oxadiazol-2-yl); (11) n=1, Z=4-COOH; (12) n=1, Z=4-CH₂COOH; (13) n=1, Z=4-(2-fluorobenzylcarbamoylmethyl).

On the other hand, as a 2-aminopyridine compound, WO01/007411 describes the following compound (tert-butyl [2-({[(9-oxo-9H-fluoren-4-yl)amino]carbonyl}amino)pyridin-4-yl]carbamate).

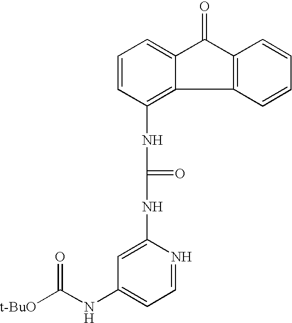

However, the above-mentioned 2-aminopyridine compound is a synthetic intermediate, and it has been known that the compound has a GSK-3 inhibitory action.

### DISCLOSURE OF THE INVENTION

### Problems to be Solved by the Invention

Conventional compounds having a GSK-3 inhibitory action have some problems to be solved, such as effectiveness (e.g., insufficient GSK-3 inhibitory action, insufficient selectivity to other kinase inhibitory action and the like), and safety (e.g., possible side effects and the like). In addition, since they are not sufficient in the property (stability, solubility and the like), oral absorbability, transferability to target organ and the like, practically satisfactory results as a pharmaceutical agent have not been achieved entirely. Thus, the development of a superior GSK-3 inhibitor effective as a pharmaceutical agent for GSK-3 related pathology or disease has been demanded.

The present invention aims at providing a safe GSK-3 inhibitor useful as an agent for the prophylaxis or treatment of GSK-3 related pathology or disease.

### Means of Solving the Problems

The present inventors have conducted intensive studies and found that the 2-aminopyridine compounds represented by the following formula (I) and salts thereof unexpectedly have a superior GSK-3 specific inhibitory activity based on their specific chemical structures, and further, superior properties of pharmaceutical product such as stability, solubility and the like, and can be safe and useful pharmaceutical agents for the prophylaxis or treatment of GSK-3 related pathology or disease in mammal, which resulted in the completion of the present invention.

Accordingly, the present invention provides the following.
[1] A compound represented by the formula (IA):

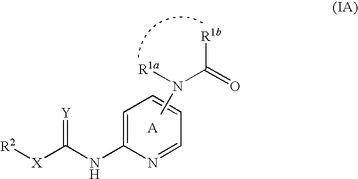

wherein
R^{1a} is a hydrogen atom, a hydrocarbon group optionally having substituent(s) or a heterocyclic group optionally having substituent(s);
R^{1b} is a hydrocarbon group optionally having substituent(s), a hydrocarbon-oxy group optionally having substituent(s) or a monocyclic heterocyclic group optionally having substituent(s); or,
R^{1a} and R^{1b} optionally form, together with the nitrogen atom and carbon atom they are bonded to, a monocyclic to tricyclic nitrogen-containing heterocycle having an oxo group and optionally having substituent(s) besides the oxo group;
R² is a hydrocarbon group optionally having substituent(s) or a heterocyclic group optionally having substituent(s);
X is an imino optionally having a substituent, —O—, —CO—NH— or a bond;
Y is an oxygen atom or a sulfur atom; and
ring A is a pyridine ring optionally further having 1 to 3 substituents selected from a halogen atom and a lower alkyl group,
or a salt thereof,
provided that tert-butyl [2-({[(9-oxo-9H-fluoren-4-yl)amino]carbonyl}amino)pyridin-4-yl]carbamate is excluded (hereinafter to be abbreviated as compound (IA));
[2] compound (IA) which is a compound represented by the formula (I):

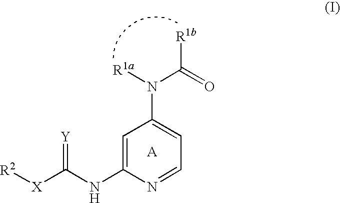

wherein each symbol is as defined in the above-mentioned [1], or a salt thereof
provided that tert-butyl [2-({[(9-oxo-9H-fluoren-4-yl)amino]carbonyl}amino)pyridin-4-yl]carbamate is excluded (hereinafter to be abbreviated as compound (I));
[3] compound (IA) wherein R^{1a} is a hydrogen atom or a hydrocarbon group optionally having substituent(s);
[4] compound (IA) wherein R^{1b} is a hydrocarbon group optionally having substituent(s) or a 5- or 6-membered aromatic heterocyclic group optionally having substituent(s);
[5] compound (IA) wherein
R^{1a} and R^{1b} optionally form, together with the nitrogen atom and carbon atom they are bonded to, a monocyclic to tricyclic nitrogen-containing heterocycle having an oxo group and optionally having substituent(s) besides the oxo group, wherein the nitrogen-containing heterocycle is
(a) a 5-membered nitrogen-containing heterocycle,
(b) a bicyclic nitrogen-containing heterocycle formed by condensation of a 5-membered nitrogen-containing heterocycle and a 6-membered aromatic ring or a C₅₋₆ cycloalkane,
(c) a bicyclic nitrogen-containing heterocycle which is a spiro ring formed by a 5-membered nitrogen-containing heterocycle and a 6-membered aromatic ring or a C₅₋₆ cycloalkane, or
(d) a tricyclic nitrogen-containing heterocycle wherein a 5-membered nitrogen-containing heterocycle and a benzene ring are condensed, and the 5-membered nitrogen-containing heterocycle and a C₅₋₆ cycloalkane form a spiro ring;
[6] compound (IA) wherein R² is a C₁₋₄ alkyl group substituted by 5- or 6-membered nitrogen-containing heterocyclic group(s);
[7] compound (IA) wherein X is an imino optionally having a substituent or a bond;
[8] compound (IA) wherein Y is an oxygen atom;
[9] compound (IA) wherein ring A is a pyridine ring without further substituent;
[10] compound (IA) which is
N-(4-(2-oxo-4-phenylpyrrolidin-1-yl)pyridin-2-yl)-N′-(pyridin-2-ylmethyl)urea,
N-(4-(2-oxo-5-phenyl-1,3-oxazolidin-3-yl)pyridin-2-yl)-N′-(pyridin-2-ylmethyl)urea,
1-(4-(6-methyl-2-oxo-1,3-benzoxazol-3(2H)-yl)pyridin-2-yl)-3-(pyridin-2-ylmethyl)urea, or
N-(2-(((pyridin-2-ylmethyl)carbamoyl)amino)pyridin-4-yl)pyridine-2-carboxamide;
[11] a prodrug of compound (IA);
[12] a pharmaceutical agent comprising compound (IA) or a prodrug thereof;
[13] the pharmaceutical agent of the above-mentioned [12], which is a GSK-3 inhibitor;
[14] the pharmaceutical agent of the above-mentioned [13], wherein the GSK-3 is GSK-3β;
[15] the pharmaceutical agent of the above-mentioned [12], which is a neural stem cell differentiation promoter;
[16] the pharmaceutical agent of the above-mentioned [12], which is an agent for the prophylaxis or treatment of neurodegenerative disease or diabetes;
[17] the pharmaceutical agent of the above-mentioned [12], which is a hypoglycemic agent;
[18] a method of inhibiting GSK-3β in a mammal, which comprises administering compound (IA) or a prodrug thereof to the mammal;
[19] the method of the above-mentioned [18], wherein the GSK-3 is GSK-3β;
[20] a method of promoting differentiation of neural stem cells in a mammal, which comprises administering compound (IA) or a prodrug thereof to the mammal;
[21] a method for the prophylaxis or treatment of neurodegenerative disease or diabetes in a mammal, which comprises administering compound (IA) or a prodrug thereof to the mammal;
[22] a method of decreasing blood glucose in a mammal, which comprises administering compound (IA) or a prodrug thereof to the mammal;
[23] use of compound (IA) or a prodrug thereof for the production of a GSK-3 inhibitor;
[24] the use of the above-mentioned [23], wherein the GSK-3 is GSK-3β;
[25] use of compound (IA) or a prodrug thereof for the production of a neural stem cell differentiation promoter;
[26] use of compound (IA) or a prodrug thereof for the production of an agent for the prophylaxis or treatment of neurodegenerative disease or diabetes;
[27] use of compound (IA) or a prodrug thereof for the production of a hypoglycemic agent;
[28] a compound represented by the formula (I″):

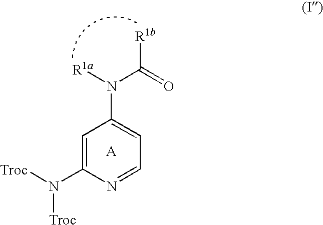

wherein
R^{1a} is a hydrogen atom or a hydrocarbon group optionally having substituent(s);
R^{1b} is a hydrocarbon group optionally having substituent(s), a hydrocarbon-oxy group optionally having substituent(s) or a 5- or 6-membered aromatic heterocyclic group optionally having substituent(s); or,
R^{1a} and R^{1b} optionally form, together with the nitrogen atom and carbon atom they are bonded to, a monocyclic to tricyclic nitrogen-containing heterocycle having an oxo group and optionally having substituent(s) besides the oxo group;
Troc is a 2,2,2-trichloroethoxycarbonyl group; and
ring A is a pyridine ring optionally further having 1 to 3 substituents selected from a halogen atom and a lower alkyl group,
or a salt thereof (hereinafter to be abbreviated as compound (I″));
and the like.

### DETAILED DESCRIPTION OF THE INVENTION

Unless otherwise specified, the term “lower” in the present specification means that the carbon number is 1 to 6.

Unless otherwise specified, examples of the “halogen atom” in the present specification include fluorine atom, chlorine atom, bromine atom, iodine atom.

Unless otherwise specified, examples of the “hydrocarbon group optionally having substituent(s)” in the present specification include a “C₁₋₆ alkyl group optionally having substituent(s)”, a “C₂₋₆ alkenyl group optionally having substituent(s)”, a “C₂₋₆ alkynyl group optionally having substituent(s)”, a “C₃₋₈ cycloalkyl group optionally having substituent(s)”, a “C₆₋₁₄ aryl group optionally having substituent(s)”, a “C₇₋₁₆ aralkyl group optionally having substituent(s)” and the like.

Unless otherwise specified, examples of the “C₁₋₆ alkyl group” in the present specification include methyl, ethyl, propyl, isopropyl, butyl, isobutyl, sec-butyl, tert-butyl, pentyl, isopentyl, neopentyl, hexyl and the like.

Unless otherwise specified, examples of the “C₂₋₆ alkenyl group” in the present specification include vinyl, propenyl, isopropenyl, 2-buten-1-yl, 4-penten-1-yl, 5-hexen-1-yl and the like.

Unless otherwise specified, examples of the “C₂₋₆ alkynyl group” in the present specification include 2-butyn-1-yl, 4-pentyn-1-yl, 5-hexyn-1-yl and the like.

Unless otherwise specified, examples of the “C₃₋₈ cycloalkyl group” in the present specification include cyclopropyl, cyclobutyl, cyclopentyl, cyclohexyl, cycloheptyl, cyclooctyl, bicyclo[2.2.1]heptyl, oxobicyclo[2.2.1]heptyl, bicyclo[2.2.2]octyl and the like.

Unless otherwise specified, examples of the “C₆₋₁₄ aryl group” in the present specification include phenyl, 1-naphthyl, 2-naphthyl, 2-biphenylyl, 3-biphenylyl, 4-biphenylyl, 2-anthryl and the like. The C₆₋₁₄ aryl group is optionally partially saturated. Examples of the partially saturated C₆₋₁₄ aryl group include indanyl, tetrahydronaphthyl and the like.

Unless otherwise specified, examples of the “C₇₋₁₆ aralkyl group” in the present specification include benzyl, 2-phenylethyl, 1-phenylethyl, diphenylmethyl, 1-naphthylmethyl, 2-naphthylmethyl, 2,2-diphenylethyl, 3-phenylpropyl, 3,3-diphenylpropyl, 4-phenylbutyl, 5-phenylpentyl, 2-biphenylylmethyl, 3-biphenylylmethyl, 4-biphenylylmethyl and the like.

Unless otherwise specified, examples of the “hydrocarbon-oxy group optionally having substituent(s)” in the present specification include a “C₁₋₆ alkoxy group optionally having substituent(s)”, a “C₂₋₆ alkenyloxy group optionally having substituent(s)”, a “C₂₋₆ alkynyloxy group optionally having substituent(s)”, a “C₃₋₈ cycloalkyloxy group optionally having substituent(s)”, a “C₆₋₁₄ aryloxy group optionally having substituent(s)”, a “C₇₋₁₆ aralkyloxy group optionally having substituent(s)” and the like.

Unless otherwise specified, examples of the “C₁₋₆ alkoxy group” in the present specification include methoxy, ethoxy, propoxy, isopropoxy, butoxy, isobutoxy, tert-butoxy, pentyloxy, hexyloxy and the like.

Unless otherwise specified, examples of the “C₂₋₆ alkenyloxy group” in the present specification include vinyloxy, propenyloxy, isopropenyloxy, 2-buten-1-yloxy, 4-penten-1-yloxy, 5-hexen-1-yloxy and the like.

Unless otherwise specified, examples of the “C₂₋₆ alkynyloxy group” in the present specification include 2-butyn-1-yloxy, 4-pentyn-1-yloxy, 5-hexyn-1-yloxy and the like.

Unless otherwise specified, examples of the “C₃₋₈ cycloalkyloxy group” in the present specification include cyclopropyloxy, cyclobutyloxy, cyclopentyloxy, cyclohexyloxy, cycloheptyloxy, cyclooctyloxy and the like.

Unless otherwise specified, examples of the “C₆₋₁₄ aryloxy group” in the present specification include phenoxy, 1-naphthyloxy, 2-naphthyloxy and the like.

Unless otherwise specified, examples of the “C₇₋₁₆ aralkyloxy group” in the present specification include benzyloxy, 2-phenylethyloxy, 1-phenylethyloxy and the like.

Unless otherwise specified, examples of the “hydroxy group optionally having a substituent” in the present specification include a “hydroxy group”, a “C₁₋₁₀ alkoxy group optionally having substituent(s)”, a “heterocyclyloxy group optionally having substituent(s)”, a “C₆₋₁₄ aryloxy group optionally having substituent(s)”, a “C₇₋₁₆ aralkyloxy group optionally having substituent(s)”, a “tri-C₁₋₆ alkyl-silyloxy group”, a “C₁₋₆ alkylsulfonyloxy group optionally having substituent(s)”, a “heterocyclylsulfonyloxy group optionally having substituent(s)” and the like.

Unless otherwise specified, examples of the “C₁₋₁₀ alkoxy group” in the present specification include heptyloxy, octyloxy, nonyloxy, decyloxy and the like, besides the above-mentioned “C₁₋₆ alkoxy group”.

Unless otherwise specified, examples of the “heterocyclyloxy group” in the present specification include a hydroxy group substituted by the below-mentioned “heterocyclic group”. Preferable examples of the heterocyclyloxy group include tetrahydropyranyloxy, thiazolyloxy, pyridyloxy, pyrazolyloxy, oxazolyloxy, thienyloxy, furyloxy, tetrahydrothiopyranyloxy, 1,1-dioxidotetrahydrothiopyranyloxy and the like.

Unless otherwise specified, examples of the “tri-C₁₋₆ alkyl-silyloxy group” in the present specification include trimethylsilyloxy, tert-butyl(dimethyl)silyloxy and the like.

Unless otherwise specified, examples of the “C₁₋₆ alkylsulfonyloxy group” in the present specification include methylsulfonyloxy, ethylsulfonyloxy and the like.

Unless otherwise specified, examples of the “heterocyclylsulfonyloxy group” in the present specification include a sulfonyloxy group to which the below-mentioned “heterocyclic group” is bonded. Preferable examples of the heterocyclylsulfonyloxy group include thienylsulfonyloxy, furylsulfonyloxy and the like.

Unless otherwise specified, examples of the “mercapto group optionally having a substituent” in the present specification include a “mercapto group”, a “C₁₋₁₀ alkylthio group optionally having substituent(s)”, a “heterocyclylthio group optionally having substituent(s)”, a “C₆₋₁₄ arylthio group optionally having substituent(s)”, a “C₇₋₁₆ aralkylthio group optionally having substituent(s)” and the like.

Unless otherwise specified, examples of the “C₁₋₆ alkylthio group” in the present specification include methylthio, ethylthio, propylthio, isopropylthio, butylthio, sec-butylthio, tert-butylthio and the like.

In addition, examples of the “C₁₋₁₀ alkylthio group” in the present specification include heptylthio, octylthio, nonylthio, decylthio and the like, besides the above-mentioned C₁₋₆ alkylthio group.

Unless otherwise specified, examples of the “heterocyclylthio group” in the present specification include a mercapto group substituted by the below-mentioned “heterocyclic group”. Preferable examples of the heterocyclylthio group include tetrahydropyranylthio, thiazolylthio, pyridylthio, pyrazolylthio, oxazolylthio, thienylthio, furylthio, tetrahydrothiopyranylthio, 1,1-dioxidotetrahydrothiopyranylthio and the like.

Unless otherwise specified, examples of the “C₆₋₁₄ arylthio group” in the present specification include phenylthio, 1-naphthylthio, 2-naphthylthio and the like.

Unless otherwise specified, examples of the “C₇₋₁₆ aralkylthio group” in the present specification include benzylthio, 2-phenylethylthio, 1-phenylethylthio and the like.

Unless otherwise specified, examples of the “heterocyclic group” in the present specification include a 5- to 14-membered (monocyclic, bicyclic or tricyclic) heterocyclic group containing, as a ring-constituting atom besides carbon atoms, one or two kind(s) of 1 to 4 hetero atoms selected from a nitrogen atom, a sulfur atom and an oxygen atom, preferably (i) a 5- to 14-membered (preferably 5- to 10-membered) aromatic heterocyclic group, (ii) a 5- to 10-membered non-aromatic heterocyclic group and the like. Of these, a 5- or 6-membered aromatic heterocyclic group is preferable.

Specific examples thereof include aromatic heterocyclic groups such as thienyl (e.g.: 2-thienyl, 3-thienyl), furyl (e.g.: 2-furyl, 3-furyl), pyridyl (e.g.: 2-pyridyl, 3-pyridyl, 4-pyridyl), thiazolyl (e.g.: 2-thiazolyl, 4-thiazolyl, 5-thiazolyl), oxazolyl (e.g.: 2-oxazolyl, 4-oxazolyl, 5-oxazolyl), oxadiazolyl (e.g.: 1,3,4-oxadiazol-2-yl), quinolyl (e.g.: 2-quinolyl, 3-quinolyl, 4-quinolyl, 5-quinolyl, 6-quinolyl, 8-quinolyl), isoquinolyl (e.g.: 1-isoquinolyl, 3-isoquinolyl, 4-isoquinolyl, 5-isoquinolyl), pyrazinyl, pyrimidinyl (e.g.: 2-pyrimidinyl, 4-pyrimidinyl), pyrrolyl (e.g.: 1-pyrrolyl, 2-pyrrolyl, 3-pyrrolyl), imidazolyl (e.g.: 1-imidazolyl, 2-imidazolyl, 4-imidazolyl), pyrazolyl (e.g.: 1-pyrazolyl, 3-pyrazolyl, 4-pyrazolyl), pyridazinyl (e.g.: 3-pyridazinyl, 4-pyridazinyl), isothiazolyl (e.g.: 3-isothiazolyl, 4-isothiazolyl, 5-isothiazolyl), isoxazolyl (e.g.: 3-isoxazolyl, 4-isoxazolyl, 5-isoxazolyl), indolyl (e.g.: 1-indolyl, 2-indolyl, 3-indolyl), indazolyl (e.g.: 1-indazolyl, 3-indazolyl, 5-indazolyl), benzothiazolyl (e.g.: 2-benzothiazolyl), benzoxazolyl (e.g.: 2-benzoxazolyl), benzimidazolyl (e.g.: 1-benzimidazolyl, 2-benzimidazolyl), benzo[b]thienyl (e.g.: 2-benzo[b]thienyl, 3-benzo[b]thienyl), benzo[b]furanyl (e.g.: 2-benzo[b]furanyl, 3-benzo[b]furanyl), benzotriazolyl (e.g.: 1-benzotriazolyl, 5-benzotriazolyl), imidazo[1,2-a]pyridinyl (e.g.: 2-imidazo[1,2-a]pyridinyl, 3-imidazo[1,2-a]pyridinyl, 6-imidazo[1,2-a]pyridinyl), imidazo[1,2-a]pyrimidinyl (e.g.: 2-imidazo[1,2-a]pyrimidinyl, 3-imidazo[1,2-a]pyrimidinyl, 5-imidazo[1,2-a]pyrimidinyl), pyrrolo[2,3-b]pyridinyl (e.g.: 2-1H-pyrrolo[2,3-b]pyridinyl, 3-1H-pyrrolo[2,3-b]pyridinyl, 4-1H-pyrrolo[2,3-b]pyridinyl), [1,2,4]triazolo[1,5-a]pyridinyl (e.g.: 2-[1,2,4]triazolo[1,5-a]pyridinyl, 6-[1,2,4]triazolo[1,5-a]pyridinyl, 7-[1,2,4]triazolo[1,5-a]pyridinyl) and the like; non-aromatic heterocyclic groups such as pyrrolidinyl (e.g.: 1-pyrrolidinyl, 2-pyrrolidinyl, 3-pyrrolidinyl), oxazolidinyl (e.g.: 2-oxazolidinyl), imidazolinyl (e.g.: 1-imidazolinyl, 2-imidazolinyl, 4-imidazolinyl), piperidinyl (e.g.: piperidino, 2-piperidinyl, 3-piperidinyl, 4-piperidinyl), piperazinyl (e.g.: 1-piperazinyl, 2-piperazinyl), morpholinyl (e.g.: 2-morpholinyl, 3-morpholinyl, morpholino), thiomorpholinyl (e.g.: 2-thiomorpholinyl, 3-thiomorpholinyl, thiomorpholino), tetrahydropyranyl (e.g.: 2-tetrahydropyranyl, 3-tetrahydropyranyl, 4-tetrahydropyranyl), tetrahydrofuranyl (e.g.: 2-tetrahydrofuranyl, 3-tetrahydrofuranyl), oxetanyl (e.g.: 2-oxetanyl, 3-oxetanyl), oxopyrrolidinyl (e.g.: 2-oxopyrrolidin-1-yl, 2-oxopyrrolidin-3-yl, 2-oxopyrrolidin-4-yl, 2-oxopyrrolidin-5-yl, 3-oxopyrrolidin-1-yl), dioxopyrrolidinyl (e.g.: 2,5-dioxopyrrolidin-1-yl, 2,5-dioxopyrrolidin-3-yl), tetrahydrothiopyranyl (e.g.: 2-tetrahydrothiopyranyl, 3-tetrahydrothiopyranyl, 4-tetrahydrothiopyranyl), 1,1-dioxidotetrahydrothiopyranyl (e.g.: 1,1-dioxidotetrahydrothiopyran-2-yl, 1,1-dioxidotetrahydrothiopyran-3-yl, 1,1-dioxidotetrahydrothiopyran-4-yl), dihydrobenzofuranyl (e.g.: 2,3-dihydro-1-benzofuran-4-yl, 2,3-dihydro-1-benzofuran-5-yl, 2,3-dihydro-1-benzofuran-6-yl, 2,3-dihydro-1-benzofuran-7-yl), benzodioxolyl (e.g.: benzodioxol-5-yl), tetrahydrobenzo[c]azepinyl (e.g.: 1,3,4,5-tetrahydrobenzo[c]azepin-2-yl), tetrahydroisoquinolyl (e.g.: 1,2,3,4-tetrahydroisoquinolin-2-yl) and the like, and the like.

Unless otherwise specified, examples of the “monocyclic heterocyclic group” in the present specification include a monocyclic heterocyclic group, from among the aforementioned “heterocyclic group”.

Unless otherwise specified, examples of the “5- or 6-membered aromatic heterocyclic group” in the present specification include a 5- or 6-membered aromatic heterocyclic group, from among the aforementioned “heterocyclic group”.

Unless otherwise specified, examples of the “C₁₋₆ alkylsulfonyl group” in the present specification include methylsulfonyl, ethylsulfonyl and the like.

Unless otherwise specified, examples of the “C₆₋₁₄ arylsulfonyl group” in the present specification include phenylsulfonyl, 1-naphthylsulfonyl, 2-naphthylsulfonyl and the like.

Unless otherwise specified, examples of the “C₇₋₁₆ aralkylsulfonyl group” in the present specification include benzylsulfonyl, 1-phenylethylsulfonyl, 2-phenylethylsulfonyl and the like.

Unless otherwise specified, examples of the “C₁₋₆ alkylsulfinyl group” in the present specification include methylsulfinyl, ethylsulfinyl and the like.

Unless otherwise specified, examples of the “C₆₋₁₄ arylsulfinyl group” in the present specification include phenylsulfinyl, 1-naphthylsulfinyl, 2-naphthylsulfinyl and the like.

Unless otherwise specified, examples of the “C₁₋₆ alkyl-carbonyl group” in the present specification include acetyl, isobutanoyl, isopentanoyl and the like.

Unless otherwise specified, examples of the “C₃₋₈ cycloalkyl-carbonyl group” in the present specification include cyclopentylcarbonyl, cyclohexylcarbonyl and the like.

Unless otherwise specified, examples of the “C₆₋₁₄ aryl-carbonyl group” in the present specification include benzoyl, 1-naphthylcarbonyl, 2-naphthylcarbonyl and the like.

Unless otherwise specified, examples of the “C₇₋₁₆ aralkyl-carbonyl group” in the present specification include phenylacetyl, 2-phenylpropanoyl and the like.

Unless otherwise specified, examples of the “heterocyclyl-carbonyl group” in the present specification include a carbonyl group which the aforementioned “heterocyclic group” is bonded to. Examples thereof include pyrrolidinylcarbonyl, piperidinocarbonyl, piperazinylcarbonyl, morpholinocarbonyl, thiomorpholinocarbonyl, thienylcarbonyl, tetrahydrobenzo[c]azepinylcarbonyl, tetrahydroisoquinolylcarbonyl and the like.

Unless otherwise specified, examples of the “optionally esterified carboxyl group” in the present specification include a carboxyl group, a C₁₋₆ alkoxy-carbonyl group, a C₆₋₁₄ aryloxy-carbonyl group, a C₇₋₁₆ aralkyloxy-carbonyl group and the like.

Unless otherwise specified, examples of the “C₁₋₆ alkoxy-carbonyl group” in the present specification include methoxycarbonyl, ethoxycarbonyl, propoxycarbonyl, tert-butoxycarbonyl and the like.

Unless otherwise specified, examples of the “C₆₋₁₄ aryloxy-carbonyl group” in the present specification include phenoxycarbonyl, naphthyloxycarbonyl and the like.

Unless otherwise specified, examples of the “C₇₋₁₆ aralkyloxy-carbonyl group” in the present specification include benzyloxycarbonyl, 2-phenylethyloxycarbonyl and the like.

Unless otherwise specified, examples of the “optionally halogenated C₁₋₆ alkyl group” in the present specification include the above-mentioned “C₁₋₆ alkyl group” optionally having 1 to 5 “halogen atoms” mentioned above. Examples thereof include methyl, ethyl, propyl, isopropyl, butyl, tert-butyl, isobutyl, trifluoromethyl and the like.

Unless otherwise specified, examples of the “optionally halogenated C₁₋₆ alkoxy group” in the present specification include the above-mentioned “C₁₋₆ alkoxy group” optionally having 1 to 5 “halogen atoms” mentioned above. Examples thereof include methoxy, ethoxy, isopropoxy, tert-butoxy, trifluoromethoxy and the like.

Unless otherwise specified, examples of the “C₁₋₆ alkoxy-C₁₋₆ alkoxy group” in the present specification include the above-mentioned “C₁₋₆ alkoxy group” optionally having 1 to 5 “C₁₋₆ alkoxy groups” mentioned above. Examples thereof include methoxymethoxy, methoxyethoxy, methoxyisopropoxy, ethoxymethoxy, ethoxyethoxy, ethoxyisopropoxy and the like.

Unless otherwise specified, examples of the “mono- or di-C₁₋₆ alkyl-amino group” in the present specification include an amino group mono- or di-substituted by the above-mentioned “C₁₋₆ alkyl group(s)”. Examples thereof include methylamino, ethylamino, propylamino, dimethylamino, diethylamino and the like.

Unless otherwise specified, examples of the “mono- or di-C₃₋₈ cycloalkyl-amino group” in the present specification include an amino group mono- or di-substituted by the above-mentioned “C₃₋₈ cycloalkyl group(s)”. Examples thereof include cyclopropylamino and the like.

Unless otherwise specified, examples of the “mono- or di-C₆₋₁₄ aryl-amino group” in the present specification include an amino group mono- or di-substituted by the above-mentioned “C₆₋₁₄ aryl group(s)”. Examples thereof include phenylamino, diphenylamino, 1-naphthylamino, 2-naphthylamino and the like.

Unless otherwise specified, examples of the “mono- or di-C₇₋₁₆ aralkyl-amino group” in the present specification include an amino group mono- or di-substituted by the above-mentioned “C₇₋₁₆ aralkyl group(s)”. Examples thereof include benzylamino, 2-phenylethylamino and the like.

Unless otherwise specified, examples of the “N—C₁₋₆ alkyl-N—C₆₋₁₄ aryl-amino group” in the present specification include an amino group substituted by the above-mentioned “C₁₋₆ alkyl group” and the above-mentioned “C₆₋₁₄ aryl group”. Examples thereof include N-methyl-N-phenylamino, N-ethyl-N-phenylamino and the like.

Unless otherwise specified, examples of the “N—C₁₋₆ alkyl-N—C₇₋₁₆ aralkyl-amino group” in the present specification include an amino group substituted by the above-mentioned “C₁₋₆ alkyl group” and the above-mentioned “C₇₋₁₆ aralkyl group”. Examples thereof include N-methyl-N-benzylamino, N-ethyl-N-benzylamino and the like.

Unless otherwise specified, examples of the “mono- or di-(C₁₋₆ alkyl-carbonyl)-amino group” in the present specification include an amino group mono- or di-substituted by the above-mentioned “C₁₋₆ alkyl-carbonyl group(s)”. Examples thereof include acetylamino, propionylamino and the like.

Unless otherwise specified, examples of the “N—C₁₋₆ alkyl-N—(C₁₋₆ alkyl-carbonyl)-amino group” in the present specification include an amino group substituted by the above-mentioned “C₁₋₆ alkyl group” and the above-mentioned “C₁₋₆ alkyl-carbonyl group”. Examples thereof include N-acetyl-N-methylamino, N-acetyl-N-ethylamino and the like.

Unless otherwise specified, examples of the “mono- or di-C₁₋₆ alkyl-carbamoyl group” in the present specification include a carbamoyl group mono- or di-substituted by the above-mentioned “C₁₋₆ alkyl group(s)”. Examples thereof include methylcarbamoyl, ethylcarbamoyl, dimethylcarbamoyl, diethylcarbamoyl, ethylmethylcarbamoyl and the like.

Unless otherwise specified, examples of the “mono- or di-C₆₋₁₄ aryl-carbamoyl group” in the present specification include a carbamoyl group mono- or di-substituted by the above-mentioned “C₆₋₁₄ aryl group(s)”. Examples thereof include phenylcarbamoyl, 1-naphthylcarbamoyl, 2-naphthylcarbamoyl and the like.

Unless otherwise specified, examples of the “mono- or di-5- to 7-membered heterocyclyl-carbamoyl group” in the present specification include a carbamoyl group mono- or di-substituted by 5- to 7-membered heterocyclic group(s). Examples of the “5- to 7-membered heterocyclic group” include a 5- to 7-membered heterocyclic group, from among the above-mentioned “heterocyclic group”. Preferable examples of the “mono- or di-5- to 7-membered heterocyclyl-carbamoyl group” include 2-pyridylcarbamoyl, 3-pyridylcarbamoyl, 4-pyridylcarbamoyl, 2-thienylcarbamoyl, 3-thienylcarbamoyl and the like.

Unless otherwise specified, examples of the “N—C₁₋₆ alkyl-N—C₁₋₆ alkoxy-carbamoyl group” in the present specification include a carbamoyl group substituted by the above-mentioned “C₁₋₆ alkyl group” and the above-mentioned “C₁₋₆ alkoxy group”. Examples thereof include N-methyl-N-methoxycarbamoyl and the like.

Unless otherwise specified, examples of the “C₃₋₈ cycloalkyl-carbamoyl group” in the present specification include a carbamoyl group mono- or di-substituted by the above-mentioned “C₃₋₈ cycloalkyl group(s)”. Examples thereof include cyclopropylcarbamoyl and the like.

Unless otherwise specified, examples of the “C₇₋₁₆ aralkyl-carbamoyl group” in the present specification include a carbamoyl group mono- or di-substituted by the above-mentioned “C₇₋₁₆ aralkyl group(s)”. Examples thereof include benzylcarbamoyl and the like.

Unless otherwise specified, examples of the “mono- or di-C₁₋₆ alkyl-sulfamoyl group” in the present specification include a sulfamoyl group mono- or di-substituted by the above-mentioned “C₁₋₆ alkyl group(s)”. Examples thereof include methylsulfamoyl, ethylsulfamoyl, dimethylsulfamoyl, diethylsulfamoyl and the like.

Unless otherwise specified, examples of the “mono- or di-C₆₋₁₄ aryl-sulfamoyl group” in the present specification include a sulfamoyl group mono- or di-substituted by the above-mentioned “C₆₋₁₄ aryl group(s)”. Examples thereof include phenylsulfamoyl, diphenylsulfamoyl, 1-naphthylsulfamoyl, 2-naphthylsulfamoyl and the like.

Unless otherwise specified, examples of the “nitrogen-containing heterocyclylsulfonyl group” in the present specification include a sulfonyl group to which a nitrogen-containing heterocyclic group is bonded. Examples of the “nitrogen-containing heterocyclic group” include a nitrogen-containing heterocyclic group, from among the above-mentioned “heterocyclic group”. Preferable examples of the “nitrogen-containing heterocyclylsulfonyl group” include pyridylsulfonyl and the like.

Unless otherwise specified, examples of the “nitrogen-containing heterocyclylthio group” in the present specification include a mercapto group substituted by a nitrogen-containing heterocyclic group. Examples of the “nitrogen-containing heterocyclic group” include a nitrogen-containing heterocyclic group, from among the above-mentioned “heterocyclic group”. Preferable examples of the “nitrogen-containing heterocyclylthio group” include pyridylthio and the like.

Unless otherwise specified, examples of the “nitrogen-containing heterocyclyl-amino group” in the present specification include an amino group substituted by nitrogen-containing heterocyclic group(s). Examples of the “nitrogen-containing heterocyclic group” include a nitrogen-containing heterocyclic group, from among the above-mentioned “heterocyclic group”. Preferable examples of the “nitrogen-containing heterocyclyl-amino group” include pyridylamino and the like.

Unless otherwise specified, examples of the “nitrogen-containing heterocyclyl-carbonyl group” in the present specification include a carbonyl group to which a nitrogen-containing heterocyclic group is bonded. Examples of the “nitrogen-containing heterocyclic group” include a nitrogen-containing heterocyclic group, from among the above-mentioned “heterocyclic group”. Preferable examples of the “nitrogen-containing heterocyclyl-carbonyl group” include pyrrolidinylcarbonyl, piperidinocarbonyl, morpholinocarbonyl, thiomorpholinocarbonyl and the like.

Examples of the “C₁₋₆ alkyl group optionally having substituent(s)”, “C₂₋₆ alkenyl group optionally having substituent(s)”, “C₂₋₆ alkynyl group optionally having substituent(s)”, “C₁₋₁₀ alkoxy group optionally having substituent(s) (including the C₁₋₆ alkoxy group optionally having substituent(s))”, “C₂₋₆ alkenyloxy group optionally having substituent(s)”, “C₂₋₆ alkynyloxy group optionally having substituent(s)”, “C₁₋₆ alkylsulfonyloxy group optionally having substituent(s)” and “C₁₋₁₀ alkylthio group optionally having substituent(s)” in the present specification include a “C₁₋₆ alkyl group”, a “C₂₋₆ alkenyl group”, a “C₂₋₆ alkynyl group”, a “C₁₋₁₀ alkoxy group (including a C₁₋₆ alkoxy group)”, a “C₂₋₆ alkenyloxy group”, a “C₂₋₆ alkynyloxy group”, a “C₁₋₆ alkylsulfonyloxy group” and a “C₁₋₁₀ alkylthio group”, each of which optionally has 1 to 5 substituents at substitutable positions selected from
(1) a halogen atom;
(2) a hydroxy group;
(3) an amino group;
(4) a nitro group;
(5) a cyano group;
(6) a heterocyclic group (preferably furyl, pyridyl, thienyl, pyrazolyl, thiazolyl, oxazolyl, isoxazolyl, isothiazolyl, oxetanyl, morpholinyl, thiomorpholinyl, pyrrolidinyl, oxopyrrolidinyl, dioxopyrrolidinyl, piperidinyl, tetrahydrofuranyl, tetrahydropyranyl, tetrahydrothiopyranyl, 1,1-dioxidotetrahydrothiopyranyl, dihydrobenzofuranyl, benzofuranyl, benzothiazolyl, benzodioxolyl, imidazo[1,2-a]pyridyl, imidazo[1,2-a]pyrimidinyl, oxadiazolyl) optionally having 1 to 3 substituents selected from a halogen atom, a hydroxy group, an amino group, a nitro group, a cyano group, a C₁₋₆ alkyl group (the C₁₋₆ alkyl group optionally has 1 to 3 substituents selected from a halogen atom, a cyano group and a C₃₋₈ cycloalkyl group), a C₂₋₆ alkenyl group (the C₂₋₆ alkenyl group optionally has C₆₋₁₄ aryl group(s) optionally having 1 to 3 halogen atoms), a mono- or di-C₁₋₆ alkyl-amino group, a C₆₋₁₄ aryl group (the C₆₋₁₄ aryl group optionally has 1 to 3 C₁₋₆ alkoxy groups), a mono- or di-C₆₋₁₄ aryl-amino group, a C₃₋₈ cycloalkyl group, a C₁₋₆ alkoxy group, a C₇₋₁₆ aralkyloxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group, a mono- or di-C₆₋₁₄ aryl-sulfamoyl group and a heterocyclic group (e.g., thienyl);
(7) a mono- or di-C₁₋₆ alkyl-amino group;
(8) a mono- or di-C₃₋₈ cycloalkyl-amino group;
(9) a mono- or di-C₆₋₁₄ aryl-amino group optionally having 1 to 3 halogen atoms;
(10) a mono- or di-C₇₋₁₆ aralkyl-amino group;
(11) an N—C₁₋₆ alkyl-N—C₆₋₁₄ aryl-amino group;
(12) an N—C₁₋₆ alkyl-N-C₇₋₁₆ aralkyl-amino group;
(13) a C₃₋₈ cycloalkyl group optionally having C₁₋₆ alkyl group(s);
(14) an optionally halogenated C₁₋₆ alkoxy group;
(15) a C₁₋₆ alkylthio group optionally having C₁₋₆ alkoxy group(s);
(16) a C₁₋₆ alkylsulfinyl group optionally having C₁₋₆ alkoxy group(s);
(17) a C₁₋₆ alkylsulfonyl group optionally having C₁₋₆ alkoxy group(s);
(18) an optionally esterified carboxyl group;
(19) a carbamoyl group;
(20) a thiocarbamoyl group;
(21) a mono- or di-C₁₋₆ alkyl-carbamoyl group;
(22) a mono- or di-C₆₋₁₄ aryl-carbamoyl group;
(23) a mono- or di-5- to 7-membered heterocyclyl-carbamoyl group;
(24) an N—C₁₋₆ alkyl-N—C₁₋₆ alkoxy-carbamoyl group;
(25) a mono- or di-(C₁₋₆ alkyl-carbonyl)-amino group optionally having carboxyl group(s);
(26) a C₆₋₁₄ aryloxy group optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(27) a C₆₋₁₄ aryl group optionally having 1 to 3 substituents selected from a halogen atom, a hydroxy group, an amino group, a nitro group, a cyano group, a C₁₋₆ alkyl group (the C₁₋₆ alkyl group optionally has 1 to 3 substituents selected from a halogen atom and a hydroxy group), a mono- or di-C₁₋₆ alkyl-amino group, a C₆₋₁₄ aryl group, a mono- or di-C₆₋₁₄ aryl-amino group, a mono- or di-(C₁₋₆ alkyl-carbonyl)-amino group, a C₃₋₈ cycloalkyl group, a C₁₋₆ alkoxy group (the C₁₋₆ alkoxy group optionally has 1 to 3 halogen atoms), a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group, a mono- or di-C₆₋₁₄ aryl-sulfamoyl group, a C₁₋₆ alkyl-carbonyl group, a heterocyclic group (e.g., pyrrolyl) and a heterocyclyl-carbonyl group (e.g., piperazinylcarbonyl, morpholinocarbonyl);
(28) a heterocyclyloxy group optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(29) a sulfamoyl group;
(30) a mono- or di-C₁₋₆ alkyl-sulfamoyl group;
(31) a mono- or di-C₆₋₁₄ aryl-sulfamoyl group;
(32) a C₇₋₁₆ aralkyloxy group optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(33) a C₁₋₆ alkylsulfonyloxy group;
(34) a tri-C₁₋₆ alkyl-silyloxy group;
(35) a heterocyclyl-carbonyl group (e.g., thienylcarbonyl, tetrahydrobenzo[c]azepinylcarbonyl, tetrahydroisoquinolylcarbonyl);
(36) a C₆₋₁₄ aryl-carbonyl group;
(37) a C₆₋₁₄ arylthio group optionally having 1 to 3 substituents selected from a halogen atom and a cyano group;
(38) a C₆₋₁₄ arylsulfinyl group optionally having 1 to 3 halogen atoms;
(39) a C₆₋₁₄ arylsulfonyl group optionally having 1 to 3 halogen atoms;
(40) a nitrogen-containing heterocyclylsulfonyl group;
(41) a nitrogen-containing heterocyclylthio group;
(42) a nitrogen-containing heterocyclyl-amino group optionally having 1 to 3 substituents selected from a cyano group and a nitro group;
and the like.

Examples of the “C₃₋₈ cycloalkyl group optionally having substituent(s)”, “C₆₋₁₄ aryl group optionally having substituent(s)”, “C₇₋₁₆ aralkyl group optionally having substituent(s)”, “heterocyclic group optionally having substituent(s) (including the monocyclic heterocyclic group optionally having substituent(s) and the 5- or 6-membered aromatic heterocyclic group optionally having substituent(s)”, “heterocyclyloxy group optionally having substituent(s)”, “C₃₋₈ cycloalkyloxy group optionally having substituent(s)”, “C₆₋₁₄ aryloxy group optionally having substituent(s)”, “C₇₋₁₆ aralkyloxy group optionally having substituent(s)”, “heterocyclylsulfonyloxy group optionally having substituent(s)”, “heterocyclylthio group optionally having substituent(s)”, “C₆₋₁₄ arylthio group optionally having substituent(s)” and “C₇₋₁₆ aralkylthio group optionally having substituent(s)” in the present specification include a “C₃₋₈ cycloalkyl group”, a “C₆₋₁₄ aryl group”, a “C₇₋₁₆ aralkyl group”, a “heterocyclic group (including a monocyclic heterocyclic group and a 5- or 6-membered aromatic heterocyclic group)”, a “heterocyclyloxy group”, a “C₃₋₈ cycloalkyloxy group”, a “C₆₋₁₄ aryloxy group”, a “C₇₋₁₆ aralkyloxy group”, a “heterocyclylsulfonyloxy group”, a “heterocyclylthio group”, a “C₆₋₁₄ arylthio group” and a “C₇₋₁₆ aralkylthio group”, each of which optionally has 1 to 5 substituents at substitutable positions selected from
(1) a halogen atom;
(2) a hydroxy group;
(3) an amino group;
(4) a nitro group;
(5) a cyano group;
(6) a C₁₋₆ alkyl group optionally having substituent(s);
(7) a C₂₋₆ alkenyl group optionally having substituent(s);
(8) a C₂₋₆ alkynyl group optionally having substituent(s);
(9) a C₆₋₁₄ aryl group optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(10) a C₆₋₁₄ aryloxy group optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(11) a C₇₋₁₆ aralkyloxy group optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(12) a heterocyclic group (preferably furyl, pyridyl, thienyl, pyrazolyl, thiazolyl, oxazolyl, isoxazolyl, isothiazolyl, oxetanyl, morpholinyl, thiomorpholinyl, pyrrolidinyl, oxopyrrolidinyl, dioxopyrrolidinyl, tetrahydropyranyl, tetrahydrothiopyranyl, 1,1-dioxidotetrahydrothiopyranyl) optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(13) a mono- or di-C₁₋₆ alkyl-amino group;
(14) a mono- or di-C₆₋₁₄ aryl-amino group;
(15) a mono- or di-C₇₋₁₆ aralkyl-amino group;
(16) an N-C₁₋₆ alkyl-N-C₆₋₁₄ aryl-amino group;
(17) an N-C₁₋₆ alkyl-N-C₇₋₁₆ aralkyl-amino group;
(18) a C₃₋₈ cycloalkyl group;
(19) a C₁₋₆ alkoxy group optionally having substituent(s);
(20) a C₁₋₆ alkylthio group optionally having C₁₋₆ alkoxy group(s);
(21) a C₁₋₆ alkylsulfinyl group optionally having C₁₋₆ alkoxy group(s);
(22) a C₁₋₆ alkylsulfonyl group optionally having C₁₋₆ alkoxy group(s);
(23) an optionally esterified carboxyl group;
(24) a carbamoyl group;
(25) a thiocarbamoyl group;
(26) a mono- or di-C₁₋₆ alkyl-carbamoyl group;
(27) a mono- or di-C₆₋₁₄ aryl-carbamoyl group;
(28) a mono- or di-5- to 7-membered heterocyclyl-carbamoyl group;
(29) a sulfamoyl group;
(30) a mono- or di-C₁₋₆ alkyl-sulfamoyl group;
(31) a mono- or di-C₆₋₁₄ aryl-sulfamoyl group;
(32) a C₁₋₆ alkylsulfonyloxy group;
(33) a tri-C₁₋₆ alkyl-silyloxy group;
(34) a C₁₋₆ alkyl-carbonyl group;
(35) a nitrogen-containing heterocyclyl-carbonyl group;
(36) a heterocyclyloxy group optionally having 1 to 3 substituents selected from a halogen atom, a hydroxy group, an amino group, a nitro group, a cyano group, an optionally halogenated C₁₋₆ alkyl group, a mono- or di-C₁₋₆ alkyl-amino group, a C₆₋₁₄ aryl group, a mono- or di-C₆₋₁₄ aryl-amino group, a C₃₋₈ cycloalkyl group, a C₁₋₆ alkoxy group, a C₁₋₆ alkoxy-C₁₋₆ alkoxy group, a C₁₋₆ alkylthio group, a C₁₋₆ alkylsulfinyl group, a C₁₋₆ alkylsulfonyl group, an optionally esterified carboxyl group, a carbamoyl group, a thiocarbamoyl group, a mono- or di-C₁₋₆ alkyl-carbamoyl group, a mono- or di-C₆₋₁₄ aryl-carbamoyl group, a sulfamoyl group, a mono- or di-C₁₋₆ alkyl-sulfamoyl group and a mono- or di-C₆₋₁₄ aryl-sulfamoyl group;
(37) a C₁₋₄ alkylenedioxy group (e.g., methylenedioxy, ethylenedioxy) optionally having substituent(s);
(38) a mono- or di-(C₁₋₆ alkyl-carbonyl)-amino group;
(39) an N—C₁₋₆ alkyl-N—(C₁₋₆ alkyl-carbonyl)-amino group;
(40) a formyl group;
and the like.

Unless otherwise specified, examples of the “amino group optionally having substituent(s)” in the present specification include an amino group optionally having 1 or 2 substituents selected from
(1) a C₁₋₆ alkyl group optionally having substituent(s);
(2) a C₂₋₆ alkenyl group optionally having substituent(s);
(3) a C₂₋₆ alkynyl group optionally having substituent(s);
(4) a C₃₋₈ cycloalkyl group optionally having substituent(s);
(5) a C₆₋₁₄ aryl group optionally having substituent(s);
(6) a C₁₋₆ alkoxy group optionally having substituent(s);
(7) an acyl group;
(8) a heterocyclic group (preferably furyl, pyridyl, thienyl, pyrazolyl, thiazolyl, oxazolyl) optionally having substituent(s);
(9) a sulfamoyl group;
(10) a mono- or di-C₁₋₆ alkyl-sulfamoyl group;
(11) a mono- or di-C₆₋₁₄ aryl-sulfamoyl group;
and the like. When the “amino group optionally having substituent(s)” is an amino group having 2 substituents, these substituents optionally form, together with the adjacent nitrogen atom, a nitrogen-containing heterocycle optionally having substituent(s). Examples of the “nitrogen-containing heterocycle” of the “nitrogen-containing heterocycle optionally having substituent(s)” include a 5- to 7-membered nitrogen-containing heterocycle containing, as a ring constituting atom besides carbon atoms, at least one nitrogen atom, and optionally further containing 1 or 2 hetero atoms selected from an oxygen atom, a sulfur atom and a nitrogen atom. Preferable examples of the nitrogen-containing heterocycle include pyrrolidine, imidazolidine, pyrazolidine, piperidine, piperazine, morpholine, thiomorpholine, thiazolidine, oxazolidine and the like.

The nitrogen-containing heterocycle optionally has 1 or 2 substituents at substitutable positions. Examples of such substituent include a hydroxy group, an optionally halogenated C₁₋₆ alkyl group, a C₆₋₁₄ aryl group, a C₇₋₁₆ aralkyl group and the like.

Unless otherwise specified, examples of the “acyl group” in the present specification include a group represented by formula: —COR⁷, —CO—OR⁷, —SO₂R⁷, —SOR⁷, —PO(OR⁷)(OR⁸), —CO—NR^{7a}R^{8a} or —CS—NR^{7a}R^{8a} wherein R⁷ and R⁸ are the same or different and each is a hydrogen atom, a hydrocarbon group optionally having substituent(s) or a heterocyclic group optionally having substituent(s), and R^{7a} and R^{8a} are the same or different and each is a hydrogen atom, a hydrocarbon group optionally having substituent(s) or a heterocyclic group optionally having substituent(s), or R^{7a} and R^{8a} optionally form, together with the adjacent nitrogen atom, a nitrogen-containing heterocycle optionally having substituent(s), and the like.

Examples of the “nitrogen-containing heterocycle” of the “nitrogen-containing heterocycle optionally having substituent(s)” formed by R^{7a} and R^{8a} together with the adjacent nitrogen atom include a 5- to 7-membered nitrogen-containing heterocycle containing, as a ring constituting atom besides carbon atoms, at least one nitrogen atom, and optionally further containing 1 or 2 hetero atoms selected from an oxygen atom, a sulfur atom and a nitrogen atom. Preferable examples of the nitrogen-containing heterocycle include pyrrolidine, imidazolidine, pyrazolidine, piperidine, piperazine, morpholine, thiomorpholine, thiazolidine, oxazolidine and the like.

The nitrogen-containing heterocycle optionally has 1 or 2 substituents at substitutable positions. Examples of such substituent include a hydroxy group, an optionally halogenated C₁₋₆ alkyl group, a C₆₋₁₄ aryl group, a C₇₋₁₆ aralkyl group and the like.

Preferable examples of the “acyl group” include
a formyl group;
a carboxyl group;
a carbamoyl group;
a C₁₋₆ alkyl-carbonyl group optionally having 1 to 3 halogen atoms;
a C₁₋₆ alkoxy-carbonyl group optionally having 1 to 3 halogen atoms;
a C₃₋₈ cycloalkyl-carbonyl group;
a C₆₋₁₄ aryl-carbonyl group;
a C₇₋₁₆ aralkyl-carbonyl group;
a C₆₋₁₄ aryloxy-carbonyl group;
a C₇₋₁₆ aralkyloxy-carbonyl group;
a mono- or di-C₁₋₆ alkyl-carbamoyl group;
a mono- or di-C₆₋₁₄ aryl-carbamoyl group;
a C₃₋₈ cycloalkyl-carbamoyl group;
a C₇₋₁₆ aralkyl-carbamoyl group;
a C₁₋₆ alkylsulfonyl group optionally having 1 to 3 halogen atoms;
a C₆₋₁₄ arylsulfonyl group optionally having nitro group(s);
a nitrogen-containing heterocyclyl-carbonyl group;
a C₁₋₆ alkylsulfinyl group optionally having 1 to 3 halogen atoms;
a C₆₋₁₄ arylsulfinyl group;
a thiocarbamoyl group;
and the like.

Unless otherwise specified, examples of the “C₁₋₄ alkylenedioxy group” of the “C₁₋₄ alkylenedioxy group optionally having substituent(s)” in the present specification include methylenedioxy, ethylenedioxy, propylenedioxy, tetrafluoroethylenedioxy and the like. The C₁₋₄ alkylenedioxy group optionally has 1 to 3 substituents at substitutable positions. Examples of such substituent include a halogen atom, a hydroxy group, an amino group, a mono- or di-C₁₋₆ alkyl-amino group, a mono- or di-C₆₋₁₄ aryl-amino group, a mono- or di-C₇₋₁₆ aralkyl-amino group, a nitro group, a cyano group, a C₁₋₆ alkoxy group, a C₁₋₆ alkylthio group and the like.

The definition of each symbol in the formula (IA), the formula (I) and the formula (I″) is described in detail in the following.

R^{1a} in the formula (IA) or the formula (I) is a hydrogen atom, a hydrocarbon group optionally having substituent(s) or a heterocyclic group optionally having substituent(s). R^{1a} is preferably a hydrogen atom or a hydrocarbon group optionally having substituent(s), particularly preferably a hydrogen atom.

R^{1a} in the formula (I″) is a hydrogen atom or a hydrocarbon group optionally having substituent(s). R^{1a} is preferably a hydrogen atom.

R^{1b} in the formula (IA) or the formula (I) is a hydrocarbon group optionally having substituent(s), a hydrocarbon-oxy group optionally having substituent(s) or a monocyclic heterocyclic group optionally having substituent(s). R^{1b} in the formula (I″) is a hydrocarbon group optionally having substituent(s), a hydrocarbon-oxy group optionally having substituent(s) or a 5- or 6-membered aromatic heterocyclic group optionally having substituent(s).

Specific examples of R^{1b} in the formula (IA), the formula
(I) or the formula (I″) include
(1) a C₆₋₁₄ aryl group (e.g., phenyl),
(2) a C₇₋₁₆ aralkyl group (e.g., benzyl),
(3) a C₁₋₆ alkoxy group (e.g., tert-butoxy),
(4) a 5- or 6-membered aromatic heterocyclic group (e.g., pyridyl)
and the like.

R^{1b} is preferably a hydrocarbon group optionally having substituent(s) and a 5- or 6-membered aromatic heterocyclic group optionally having substituent(s). The hydrocarbon group optionally having substituent(s) is preferably a C₆₋₁₄ aryl group, particularly preferably phenyl. The 5- or 6-membered aromatic heterocyclic group optionally having substituent(s) is preferably a 6-membered nitrogen-containing aromatic heterocyclic group, particularly preferably pyridyl.

Alternatively, R^{1a} and R^{1b} in the formula (IA), the formula (I) or the formula (I″) optionally form, together with the nitrogen atom and carbon atom they are bonded to, a monocyclic to tricyclic nitrogen-containing heterocycle having an oxo group and optionally having substituent(s) besides the oxo group.

Examples of the monocyclic to tricyclic nitrogen-containing heterocycle having an oxo group formed by R^{1a} and R^{1b} include a monocyclic to tricyclic heterocycle containing, as a ring constituting atom besides carbon atoms, at least one nitrogen atom, and optionally further containing 1 to 4 hetero atoms selected from an oxygen atom, a sulfur atom and a nitrogen atom, wherein the carbon atom adjacent to the nitrogen atom is substituted by an oxo group.

Examples of the monocyclic nitrogen-containing heterocycle having an oxo group include a 5- to 7-membered monocyclic nitrogen-containing heterocycle having an oxo group, containing, as a ring constituting atom besides carbon atoms, at least one nitrogen atom, and optionally further containing 1 to 4 hetero atoms selected from an oxygen atom, a sulfur atom and a nitrogen atom. Specific examples include pyrrolidin-2-one, 1,3-oxazolidin-2-one, imidazolidin-2-one, 1,3-dihydropyrrol-2-one, 3H-1,3-oxazol-2-one, 1,3-dihydroimidazol-2-one, 3,4-dihydro-¹H-pyridin-2-one, 4H-1,4-oxazin-3-one, azepan-2-one, 1,4-oxazepan-3-one, 1,4-thiazepan-3-one, 1,3-diazepan-2-one and the like.

The monocyclic nitrogen-containing heterocycle having an oxo group is preferably a 5-membered nitrogen-containing heterocycle having an oxo group, more preferably pyrrolidin-2-one, 1,3-oxazolidin-2-one or 3H-1,3-oxazol-2-one.

Examples of the bicyclic nitrogen-containing heterocycle having an oxo group include a bicyclic nitrogen-containing heterocycle having an oxo group, containing, as a ring constituting atom besides carbon atoms, at least one nitrogen atom, and optionally further containing 1 to 4 hetero atoms selected from an oxygen atom, a sulfur atom and a nitrogen atom. Examples thereof include (i) a bicyclic nitrogen-containing heterocycle formed by condensation of the above-mentioned monocyclic nitrogen-containing heterocycle having an oxo group and an aromatic ring, (ii) a bicyclic nitrogen-containing heterocycle formed by condensation of the above-mentioned monocyclic nitrogen-containing heterocycle having an oxo group and a non-aromatic ring, (iii) a bicyclic nitrogen-containing heterocycle which is a spiro ring formed by the above-mentioned monocyclic nitrogen-containing heterocycle having an oxo group and a non-aromatic ring, and the like. Examples of the aromatic ring include a benzene ring; a 5- or 6-membered aromatic heterocycle containing, as a ring constituting atom besides carbon atoms, 1 to 3 hetero atoms selected from an oxygen atom, a sulfur atom and a nitrogen atom (e.g., pyridine, thiophene, furan, pyrimidine etc.) and the like. Examples of the non-aromatic ring include a C₃₋₈ cycloalkane (e.g., cyclopropane, cyclobutane, cyclopentane, cyclohexane, cycloheptane etc.); a 5- or 6-membered non-aromatic heterocycle containing, as a ring constituting atom besides carbon atoms, 1 to 3 hetero atoms selected from an oxygen atom, a sulfur atom and a nitrogen atom (e.g., pyrrolidine, tetrahydropyran, tetrahydrofuran etc.) and the like. Specific examples of the bicyclic nitrogen-containing heterocycle having an oxo group include 1,3-dihydroindol-2-one, 1,3-dihydrobenzimidazol-2-one, 3H-benzoxazol-2-one, 3,4-dihydro-1H-quinolin-2-one, 2,3-dihydro-4H-1,4-benzoxazin-3-one, 1,3-dihydropyrrolo[3,2-b]pyridin-2-one, 2-azaspiro[4.5]decan-3-one and the like.

The bicyclic nitrogen-containing heterocycle having an oxo group is preferably (i) a bicyclic nitrogen-containing heterocycle formed by condensation of a 5-membered nitrogen-containing heterocycle having an oxo group and a 6-membered aromatic ring, (ii) a bicyclic nitrogen-containing heterocycle formed by condensation of a 5-membered nitrogen-containing heterocycle having an oxo group and a C₅₋₆ cycloalkane, or (iii) a bicyclic nitrogen-containing heterocycle which is a spiro ring formed by a 5-membered nitrogen-containing heterocycle having an oxo group and a C₅₋₆ cycloalkane, more preferably 1,3-dihydroindol-2-one, 1,3-dihydrobenzimidazol-2-one, 3H-benzoxazol-2-one or 2-azaspiro[4.5]decan-3-one.

Examples of the tricyclic nitrogen-containing heterocycle having an oxo group include a tricyclic nitrogen-containing heterocycle having an oxo group, containing, as a ring constituting atom besides carbon atoms, at least one nitrogen atom, and optionally further containing 1 to 4 hetero atoms selected from an oxygen atom, a sulfur atom and a nitrogen atom. Examples thereof include (i) a tricyclic nitrogen-containing heterocycle formed by condensation of the above-mentioned bicyclic nitrogen-containing heterocycle having an oxo group and an aromatic ring, (ii) a tricyclic nitrogen-containing heterocycle formed by condensation of the above-mentioned bicyclic nitrogen-containing heterocycle having an oxo group and a non-aromatic ring, (iii) a tricyclic nitrogen-containing heterocycle which is a spiro ring formed by the above-mentioned bicyclic nitrogen-containing heterocycle having an oxo group and a non-aromatic ring, and the like. Examples of the aromatic ring and non-aromatic ring include those similar to the aromatic ring and non-aromatic ring which are condensed with a monocyclic nitrogen-containing heterocycle, or which form, together with a monocyclic nitrogen-containing heterocycle, a spiro ring, exemplified for the above-mentioned bicyclic nitrogen-containing heterocycle having an oxo group. Specific examples of the tricyclic nitrogen-containing heterocycle having an oxo group include spiro[cyclopentane-1,3′-dihydroindole]-2′(1′H)-one and the like.

The tricyclic nitrogen-containing heterocycle having an oxo group is preferably a tricyclic nitrogen-containing heterocycle formed by condensation of a 5-membered nitrogen-containing heterocycle having an oxo group and a benzene ring, wherein the 5-membered nitrogen-containing heterocycle forms, together with a C₅₋₆ cycloalkane, a Spiro ring, more preferably spiro[cyclopentane-1,3′-dihydroindole]-2′(1′H)-one wherein 1,3-dihydroindol-2-one and cyclopentane form a spiro ring.

The monocyclic to tricyclic nitrogen-containing heterocycle having an oxo group is preferably
(a) a 5-membered nitrogen-containing heterocycle,
(b) a bicyclic nitrogen-containing heterocycle formed by condensation of a 5-membered nitrogen-containing heterocycle and a 6-membered aromatic ring or a C₅₋₆ cycloalkane,
(c) a bicyclic nitrogen-containing heterocycle which is a spiro ring formed by a 5-membered nitrogen-containing heterocycle and a C₅₋₆ cycloalkane, or
(d) a tricyclic nitrogen-containing heterocycle wherein a 5-membered nitrogen-containing heterocycle and a benzene ring are condensed, and the 5-membered nitrogen-containing heterocycle and a C₅₋₆ cycloalkane form a spiro ring.

The monocyclic to tricyclic nitrogen-containing heterocycle having an oxo group formed by R^{1a} and R^{1b} optionally has 1 to 5 substituents, besides the oxo group. Examples of such substituent include a hydrocarbon group optionally having substituent(s), a heterocyclic group optionally having substituent(s), a hydroxy group optionally having a substituent, an amino group optionally having substituent(s), a mercapto group optionally having a substituent, an acyl group and the like.

Specific examples of the substituent include
(1) a C₁₋₆ alkyl group (e.g., methyl, tert-butyl) optionally having 1 to 3 substituents selected from
   (a) a C₆₋₁₄ aryloxy group (e.g., phenoxy),
   (b) a C₇₋₁₆ aralkyloxy group (e.g., benzyloxy), and the like;

(2) a C₆₋₁₄ aryl group (e.g., phenyl) optionally having 1 to 3 substituents selected from
(a) a halogen atom (e.g., fluorine atom),
(b) a C₁₋₆ alkyl group (e.g., methyl) optionally having 1 to 3 halogen atoms (e.g., fluorine atom),
(c) a C₁₋₆ alkoxy group (e.g., methoxy) and the like;
(3) a C₇₋₁₆ aralkyl group (e.g., benzyl, 2-phenylethyl) optionally having 1 to 3 C₁₋₆ alkoxy groups (e.g., methoxy) and the like;
(4) a heterocyclic group (e.g., thienyl),
(5) a C₁₋₆ alkoxy-carbonyl group (e.g., ethoxycarbonyl) and the like. When the number of the substituents is not less than 2, respective substituents may be the same or different.

The substituent which the monocyclic nitrogen-containing heterocyclic group having an oxo group optionally has is preferably a C₆₋₁₄ aryl group optionally having substituent(s), a C₇₋₁₆ aralkyl group, a C₆₋₁₀ aryloxy-methyl group or a C₇₋₁₆ aralkyloxy-methyl group, particularly preferably a phenyl group optionally having substituent(s).

The substituent which the bicyclic nitrogen-containing heterocyclic group having an oxo group optionally has is preferably a C₁₋₆ alkyl group, particularly preferably a methyl group.

The tricyclic nitrogen-containing heterocyclic group having an oxo group is preferably unsubstituted.

R² in the formula (IA) or the formula (I) is a hydrocarbon group optionally having substituent(s) or a heterocyclic group optionally having substituent(s).

Specific examples of R² include
(1) a C₁₋₆ alkyl group (e.g., methyl, ethyl, propyl, tert-butyl) optionally having 1 to 3 substituents selected from
   (a) a halogen atom (e.g., chlorine atom),
   (b) a cyano group,
   (c) a heterocyclic group (e.g., piperidinyl, pyridyl, furyl, thienyl, tetrahydrofuranyl, benzothiazolyl) optionally having 1 to 3 C₁₋₆ alkyl groups (e.g., methyl) and the like,
   (d) a C₁₋₆ alkoxy group (e.g., isopropoxy),
   (e) a C₁₋₆ alkylthio group (e.g., methylthio),
   (f) a C₆₋₁₄ aryloxy group (e.g., phenoxy),
   (g) a nitrogen-containing heterocyclylthio group (e.g., pyridylthio),
   (h) a nitrogen-containing heterocyclyl-amino group (e.g., pyridylamino) optionally having nitro group(s) and the like;
(2) C₂₋₆ alkenyl group (e.g., vinyl) optionally having 1 to 3 C₆₋₁₄ aryl groups (e.g., phenyl) and the like;
(3) a C₃₋₈ cycloalkyl group (e.g., cyclohexyl)
(4) a C₆₋₁₄ aryl group (e.g., phenyl) optionally having 1 to 3 substituents selected from
   (a) a halogen atom (e.g., fluorine atom),
   (b) a cyano group,
   (c) a C₁₋₆ alkyl group (e.g., ethyl),
   (d) a C₇₋₁₆ aralkyloxy group (e.g., benzyloxy),
   (e) a C₁₋₆ alkoxy group (e.g., methoxy),
   (f) a C₁₋₆ alkyl-carbonyl group (e.g., acetyl) and the like;
(5) a C₇₋₁₆ aralkyl group (e.g., benzyl, 2-phenylethyl, 3-phenylpropyl) optionally having 1 to 3 substituents selected from
   (a) a halogen atom (e.g., fluorine atom, chlorine atom),
   (b) a C₁₋₆ alkoxy group (e.g., methoxy),
   (c) a C₁₋₄ alkylenedioxy group (e.g., methylenedioxy) and the like;
(6) a heterocyclic group (e.g., pyridyl, thienyl, pyrazolyl, isoxazolyl) optionally having 1 to 3 substituents selected from
   (a) a C₁₋₆ alkyl group (e.g., methyl),
   (b) a C₆₋₁₄ aryl group (e.g., phenyl)
   and the like
and the like.

Another specific examples of R² include
(1) a C₁₋₆ alkyl group (e.g., methyl, ethyl, propyl, isopropyl, tert-butyl, neopentyl) optionally having 1 to 3 substituents selected from
   (a) a halogen atom (e.g., chlorine atom),
   (b) a cyano group,
   (c) a heterocyclic group (e.g., piperidinyl, pyridyl, furyl, thienyl, tetrahydrofuranyl, benzothiazolyl) optionally having 1 to 3 C₁₋₆ alkyl groups (e.g., methyl) and the like,
   (d) a C₁₋₆ alkoxy group (e.g., isopropoxy),
   (e) a C₁₋₆ alkylthio group (e.g., methylthio),
   (f) a C₆₋₁₄ aryloxy group (e.g., phenoxy),
   (g) a nitrogen-containing heterocyclylthio group (e.g., pyridylthio),
   (h) a nitrogen-containing heterocyclyl-amino group (e.g., pyridylamino) optionally having nitro group(s),
   (i) a C₁₋₆ alkoxy-carbonyl group (e.g., methoxycarbonyl, ethoxycarbonyl)
   and the like;
(2) a C₂₋₆ alkenyl group (e.g., vinyl) optionally having 1 to 3 C₆₋₁₄ aryl groups (e.g., phenyl) and the like;
(3) a C₃₋₈ cycloalkyl group (e.g., cyclohexyl, cyclopropyl, cyclopentyl) optionally having 1 to 3 C₆₋₁₄ aryl groups (e.g., phenyl) and the like;
(4) a C₆₋₁₄ aryl group (e.g., phenyl, 2-biphenylyl) optionally having 1 to 3 substituents selected from
   (a) a halogen atom (e.g., fluorine atom),
   (b) a cyano group,
   (c) a C₁₋₆ alkyl group (e.g., ethyl, methyl, tert-butyl) optionally having 1 to 3 halogen atoms (e.g., fluorine atom) and the like,
   (d) a C₇₋₁₆ aralkyloxy group (e.g., benzyloxy),
   (e) a C₁₋₆ alkoxy group (e.g., methoxy),
   (f) a C₁₋₆ alkyl-carbonyl group (e.g., acetyl),
   (g) a C₁₋₄ alkylenedioxy group (e.g., ethylenedioxy),
   (h) a nitro group
   and the like;
(5) a C₇₋₁₆ aralkyl group (e.g., benzyl, 2-phenylethyl, 3-phenylpropyl) optionally having 1 to 3 substituents selected from
   (a) a halogen atom (e.g., fluorine atom, chlorine atom),
   (b) a C₁₋₆ alkoxy group (e.g., methoxy),
   (c) a C₁₋₄ alkylenedioxy group (e.g., methylenedioxy) and the like;
(6) a heterocyclic group (e.g., pyridyl, thienyl, pyrazolyl, isoxazolyl) optionally having 1 to 3 substituents selected from
   (a) a C₁₋₆ alkyl group (e.g., methyl),
   (b) a C₆₁₄ aryl group (e.g., phenyl)
   and the like
and the like.

R² is preferably a C₁₋₄ alkyl group substituted by 5- or 6-membered nitrogen-containing heterocyclic group(s), more preferably a C₁₋₄ alkyl group substituted by pyridyl or benzothiazolyl, particularly preferably 2-pyridylmethyl group.

X in the formula (IA) or the formula (I) is an imino (—NH—) optionally having a substituent, —O—, —CO—NH— or a bond.

The imino optionally has a hydrocarbon group optionally having substituent(s), a heterocyclic group optionally having substituent(s), a hydroxy group optionally having a substituent, an amino group optionally having substituent(s), a mercapto group optionally having a substituent, an acyl group and the like, and it is preferably unsubstituted.

X is preferably an imino (—NH—) optionally having a substituent or a bond, particularly preferably an imino (—NH—) or a bond.

Y in the formula (IA) or the formula (I) is an oxygen atom or a sulfur atom.

Y is preferably an oxygen atom.

Ring A in the formula (IA), the formula (I) or the formula (I″) is a pyridine ring optionally further having 1 to 3 substituents selected from a halogen atom and a lower alkyl group.

Ring A is preferably a pyridine ring without further substituent.

Of compound (IA), compound (I) is preferable. Specific examples of compound (I) include the following compounds.

### [Compound (I)-A]

Compound (I) wherein
R^{1a} is a hydrogen atom;
R^{1b} is
(1) a C₆₋₁₄ aryl group (e.g., phenyl),
(2) a C₇₋₁₆ aralkyl group (e.g., benzyl),
(3) a C₁₋₆ alkoxy group (e.g., tert-butoxy), or
(4) a 5- or 6-membered aromatic heterocyclic group (e.g., pyridyl); or
R^{1a} and R^{1b} optionally form, together with the nitrogen atom and carbon atom they are bonded to, a monocyclic to tricyclic nitrogen-containing heterocycle having an oxo group (e.g., pyrrolidin-2-one, 1,3-oxazolidin-2-one, 3H-1,3-oxazol-2-one, 1,3-dihydroindol-2-one, 1,3-dihydrobenzimidazol-2-one, 3H-benzoxazol-2-one, 3,4-dihydro-1H-quinolin-2-one, 2,3-dihydro-4H-1,4-benzoxazin-3-one, 1,3-dihydropyrrolo[3,2-b]pyridin-2-one, 2-azaspiro[4.5]decan-3-one, spiro[cyclopentane-1,3′-dihydroindole]-2′(1′H)-one) and optionally having, besides the oxo group, 1 to 3 substituents selected from
(1) a C₁₋₆ alkyl group (e.g., methyl, tert-butyl) optionally having 1 to 3 substituents selected from
   (a) a C₆₋₁₄ aryloxy group (e.g., phenoxy), and
   (b) a C₇₋₁₆ aralkyloxy group (e.g., benzyloxy);
(2) a C₆₋₁₄ aryl group (e.g., phenyl) optionally having 1 to 3 substituents selected from
   (a) a halogen atom (e.g., fluorine atom),
   (b) a C₁₋₆ alkyl group (e.g., methyl) optionally having 1 to 3 halogen atoms (e.g., fluorine atom), and
   (c) a C₁₋₆ alkoxy group (e.g., methoxy);
(3) a C₇₋₁₆ aralkyl group (e.g., benzyl, 2-phenylethyl) optionally having 1 to 3 C₁₋₆ alkoxy groups (e.g., methoxy);
(4) a heterocyclic group (e.g., thienyl), and
(5) a C₁₋₆ alkoxy-carbonyl group (e.g., ethoxycarbonyl);
R² is
(1) a C₁₋₆ alkyl group (e.g., methyl, ethyl, propyl, tert-butyl) optionally having 1 to 3 substituents selected from
   (a) a halogen atom (e.g., chlorine atom),
   (b) a cyano group,
   (c) a heterocyclic group (e.g., piperidinyl, pyridyl, furyl, thienyl, tetrahydrofuranyl, benzothiazolyl) optionally having 1 to 3 C₁₋₆ alkyl groups (e.g., methyl),
   (d) a C₁₋₆ alkoxy group (e.g., isopropoxy),
   (e) a C₁₋₆ alkylthio group (e.g., methylthio),
   (f) a C₆₋₁₄ aryloxy group (e.g., phenoxy),
   (g) a nitrogen-containing heterocyclylthio group (e.g., pyridylthio), and
   (h) a nitrogen-containing heterocyclyl-amino group (e.g., pyridylamino) optionally having nitro group(s);
(2) C₂₋₆ alkenyl group (e.g., vinyl) optionally having 1 to 3 C₆₋₁₄ aryl groups (e.g., phenyl);
(3) a C₃₋₈ cycloalkyl group (e.g., cyclohexyl)
(4) a C₆₋₁₄ aryl group (e.g., phenyl) optionally having 1 to 3 substituents selected from
   (a) a halogen atom (e.g., fluorine atom),
   (b) a cyano group,
   (c) a C₁₋₆ alkyl group (e.g., ethyl),
   (d) a C₇₋₁₆ aralkyloxy group (e.g., benzyloxy),
   (e) a C₁₋₆ alkoxy group (e.g., methoxy), and
   (f) a C₁₋₆ alkyl-carbonyl group (e.g., acetyl);
(5) a C₇₋₁₆ aralkyl group (e.g., benzyl, 2-phenylethyl, 3-phenylpropyl) optionally having 1 to 3 substituents selected from
   (a) a halogen atom (e.g., fluorine atom, chlorine atom),
   (b) a C₁₋₆ alkoxy group (e.g., methoxy), and
   (c) a C₁₋₄ alkylenedioxy group (e.g., methylenedioxy); or
(6) a heterocyclic group (e.g., pyridyl, thienyl, pyrazolyl, isoxazolyl) optionally having 1 to 3 substituents selected from
   (a) a C₁₋₆ alkyl group (e.g., methyl), and
   (b) a C₆₋₁₄ aryl group (e.g., phenyl);
X is an imino (—NH—), —O—, —CO—NH— or a bond;
Y is an oxygen atom or a sulfur atom; and
ring A is a pyridine ring without further substituent.

### [Compound (I)-B]

Compound (I) wherein
R^{1a} is a hydrogen atom;
R^{1b} is
(1) a C₆₋₁₄ aryl group (e.g., phenyl),
(2) a C₇₋₁₆ aralkyl group (e.g., benzyl),
(3) a C₁₋₆ alkoxy group (e.g., tert-butoxy), or
(4) a 5- or 6-membered aromatic heterocyclic group (e.g., pyridyl); or
R^{1a} and R^{1b} optionally form, together with the nitrogen atom and carbon atom they are bonded to, a monocyclic to tricyclic nitrogen-containing heterocycle having an oxo group (e.g., pyrrolidin-2-one, 1,3-oxazolidin-2-one, 3H-1,3-oxazol-2-one, 1,3-dihydroindol-2-one, 1,3-dihydrobenzimidazol-2-one, 3H-benzoxazol-2-one, 3,4-dihydro-1H-quinolin-2-one, 2,3-dihydro-4H-1,4-benzoxazin-3-one, 1,3-dihydropyrrolo[3,2-b]pyridin-2-one, 2-azaspiro[4.5]decan-3-one, spiro[cyclopentane-1,3′-dihydroindole]-2′(1′H)-one) and optionally having, besides the oxo group, 1 to 3 substituents selected from
(1) a C₁₋₆ alkyl group (e.g., methyl, tert-butyl) optionally having 1 to 3 substituents selected from
   (a) a C₆₋₁₄ aryloxy group (e.g., phenoxy), and
   (b) a C₇₋₁₆ aralkyloxy group (e.g., benzyloxy);
(2) a C₆₋₁₄ aryl group (e.g., phenyl) optionally having 1 to 3 substituents selected from
   (a) a halogen atom (e.g., fluorine atom),
   (b) a C₁₋₆ alkyl group (e.g., methyl) optionally having 1 to 3 halogen atoms (e.g., fluorine atom), and
   (c) a C₁₋₆ alkoxy group (e.g., methoxy);
(3) a C₇₋₁₆ aralkyl group (e.g., benzyl, 2-phenylethyl) optionally having 1 to 3 C₁₋₆ alkoxy groups (e.g., methoxy);
(4) a heterocyclic group (e.g., thienyl), and
(5) a C₁₋₆ alkoxy-carbonyl group (e.g., ethoxycarbonyl);
R² is
(1) a C₁₋₆ alkyl group (e.g., methyl, ethyl, propyl, isopropyl, tert-butyl, neopentyl) optionally having 1 to 3 substituents selected from
   (a) a halogen atom (e.g., chlorine atom),
   (b) a cyano group,
   (c) a heterocyclic group (e.g., piperidinyl, pyridyl, furyl, thienyl, tetrahydrofuranyl, benzothiazolyl) optionally having 1 to 3 C₁₋₆ alkyl groups (e.g., methyl),
   (d) a C₁₋₆ alkoxy group (e.g., isopropoxy),
   (e) a C₁₋₆ alkylthio group (e.g., methylthio),
   (f) a C₆₋₁₄ aryloxy group (e.g., phenoxy),
   (g) a nitrogen-containing heterocyclylthio group (e.g., pyridylthio),
   (h) a nitrogen-containing heterocyclyl-amino group (e.g., pyridylamino) optionally having nitro group(s), and
   (i) a C₁₋₆ alkoxy-carbonyl group (e.g., methoxycarbonyl, ethoxycarbonyl);
(2) a C₂₋₆ alkenyl group (e.g., vinyl) optionally having 1 to 3 C₆₋₁₄ aryl groups (e.g., phenyl);
(3) a C₃₋₈ cycloalkyl group (e.g., cyclohexyl, cyclopropyl, cyclopentyl) optionally having 1 to 3 C₆₋₁₄ aryl groups (e.g., phenyl);
(4) a C₆₋₁₄ aryl group (e.g., phenyl, 2-biphenylyl) optionally having 1 to 3 substituents selected from
   (a) a halogen atom (e.g., fluorine atom),
   (b) a cyano group,
   (c) a C₁₋₆ alkyl group (e.g., ethyl, methyl, tert-butyl) optionally having 1 to 3 halogen atoms (e.g., fluorine atom),
   (d) a C₇₋₁₆ aralkyloxy group (e.g., benzyloxy),
   (e) a C₁₋₆ alkoxy group (e.g., methoxy),
   (f) a C₁₋₆ alkyl-carbonyl group (e.g., acetyl),
   (g) a C₁₋₄ alkylenedioxy group (e.g., ethylenedioxy), and
   (h) a nitro group;
(5) a C₇₋₁₆ aralkyl group (e.g., benzyl, 2-phenylethyl, 3-phenylpropyl) optionally having 1 to 3 substituents selected from
   (a) a halogen atom (e.g., fluorine atom, chlorine atom),
   (b) a C₁₋₆ alkoxy group (e.g., methoxy), and
   (c) a C₁₋₄ alkylenedioxy group (e.g., methylenedioxy); or
(6) a heterocyclic group (e.g., pyridyl, thienyl, pyrazolyl, isoxazolyl) optionally having 1 to 3 substituents selected from
   (a) a C₁₋₆ alkyl group (e.g., methyl), and
   (b) a C₆₋₁₄ aryl group (e.g., phenyl);
X is an imino (—NH—), —O—, —CO—NH— or a bond;
Y is an oxygen atom or a sulfur atom; and
ring A is a pyridine ring without further substituent.

Compound (I) or (IA) which is not tert-butyl [2-({[(9-oxo-9H-fluoren-4-yl)amino]carbonyl}amino)pyridin-4-yl]carbamate is preferable.

### [Compound (I)-C]

Compound (I) which is
N-(4-(2-oxo-4-phenylpyrrolidin-1-yl)pyridin-2-yl)-N′-(pyridin-2-ylmethyl)urea (Example 4),
N-(4-(2-oxo-5-phenyl-1,3-oxazolidin-3-yl)pyridin-2-yl)-N′-(pyridin-2-ylmethyl)urea (Example 42),
1-(4-(6-methyl-2-oxo-1,3-benzoxazol-3(2H)-yl)pyridin-2-yl)-3-(pyridin-2-ylmethyl)urea (Example 65), or
N-(2-(((pyridin-2-ylmethyl)carbamoyl)amino)pyridin-4-yl)pyridine-2-carboxamide (Example 70).

Specific examples of compound (I″) include the following compounds.

### [Compound (I″) -A]

Compound (I″) wherein
R^{1a} is a hydrogen atom;
R^{1b} is a C₁₋₆ alkoxy group (e.g., tert-butoxy); or
R^{1a} and R^{1b} optionally form, together with the nitrogen atom and carbon atom they are bonded to, a monocyclic to tricyclic nitrogen-containing heterocycle having an oxo group (e.g., pyrrolidin-2-one) and optionally having, besides the oxo group, 1 to 3 substituents selected from
(1) a C₁₋₆ alkyl group (e.g., methyl) optionally having 1 to 3 C₆₋₁₄ aryloxy groups (e.g., phenoxy);
(2) a C₆₋₁₄ aryl group (e.g., phenyl) optionally having 1 to 3 substituents selected from
   (a) a halogen atom (e.g., fluorine atom),
   (b) a C₁₋₆ alkyl group (e.g., methyl) optionally having 1 to 3 halogen atoms (e.g., fluorine atom), and
   (c) a C₁₋₆ alkoxy group (e.g., methoxy); and
(3) a C₇₋₁₆ aralkyl group (e.g., benzyl, 2-phenylethyl) optionally having 1 to 3 C₁₋₆ alkoxy groups (e.g., methoxy);
Troc is a 2,2,2-trichloroethoxycarbonyl group; and
ring A is a pyridine ring without further substituent.

Examples of the salt of compound (IA), compound (I) and compound (I″) of the present invention (hereinafter to be collectively abbreviated as compound (IA)) include metal salts, ammonium salts, salts with organic bases, salts with inorganic bases, salts with organic acids, salts with basic or acidic amino acids and the like.

Preferable examples of metal salt include alkali metal salts such as sodium salt, potassium salt and the like; alkaline earth metal salts such as calcium salt, magnesium salt, barium salt and the like, and the like. Preferable examples of the salt with organic base include a salt with trimethylamine, triethylamine, pyridine, picoline, 2,6-lutidine, ethanolamine, diethanolamine, triethanolamine, cyclohexylamine, dicyclohexylamine, N,N′-dibenzylethylenediamine and the like. Preferable examples of the salt with inorganic acid include a salt with hydrochloric acid, hydrobromic acid, nitric acid, sulfuric acid, phosphoric acid and the like. Preferable examples of the salt with organic acid include a salt with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like. Preferable examples of the salt with basic amino acid include a salt with arginine, lysin, ornithine and the like. Preferable examples of the salt with acidic amino acid include a salt with aspartic acid, glutamic acid and the like.

Of these, the salt of compound (IA) is preferably a pharmaceutically acceptable salt. For example, when compound (IA) contains an acidic functional group, metal salts such as alkali metal salt, alkaline earth metal salt and the like, ammonium salts and the like are preferable. When compound (IA) contains a basic functional group, for example, salts with inorganic acid, and salts with organic acid are preferable.

A prodrug of compound (IA) means a compound which is converted to compound (IA) with a reaction due to an enzyme, an gastric acid, etc. under the physiological condition in the living body, that is, a compound which is converted to compound (IA) with oxidation, reduction, hydrolysis, etc. according to an enzyme; a compound which is converted to compound (IA) by hydrolysis etc. due to gastric acid, etc.

Examples of a prodrug of compound (IA) include a compound wherein an amino group of compound (IA) is acylated, alkylated or phosphorylated (e.g., compound wherein amino group of compound (IA) is eicosanoylated, alanylated, pentylaminocarbonylated, (5-methyl-2-oxo-1,3-dioxolen-4-yl)methoxycarbonylated, tetrahydrofuranylated, pyrrolidylmethylated, pivaloyloxymethylated or tert-butylated, and the like); a compound wherein a hydroxy group of compound (IA) is acylated, alkylated, phosphorylated or borated (e.g., a compound wherein a hydroxy group of compound (IA) is acetylated, palmitoylated, propanoylated, pivaloylated, succinylated, fumarylated, alanylated or dimethylaminomethylcarbonylated, and the like); a compound wherein a carboxyl group of compound (IA) is esterified or amidated (e.g., a compound wherein a carboxyl group of compound (IA) is ethyl esterified, phenyl esterified, carboxymethyl esterified, dimethylaminomethyl esterified, pivaloyloxymethyl esterified, ethoxycarbonyloxyethyl esterified, phthalidyl esterified, (5-methyl-2-oxo-1,3-dioxolen-4-yl)methyl esterified, cyclohexyloxycarbonylethyl esterified or methylamidated, and the like) and the like.

These compounds can be produced from compound (IA) by a method known per se.

A prodrug of compound (IA) may also be one which is converted into compound (IA) under a physiological condition, such as those described in IYAKUHIN no KAIHATSU (Development of Pharmaceuticals), Vol. 7, Design of Molecules, p. 163-198, Published by HIROKAWA SHOTEN (1990).

Hereinafter, the production methods of the compound (IA) and the like of the present invention are explained.

The compound (IA) and the like of the present invention can be produced according to the following method or a method analogous thereto. However, the production method is not limited to those described below.

Each symbol of the compounds in the schematic drawings of the following reaction schemes is as defined above unless otherwise specified. Each compound described in the reaction schemes may form a salt, and Examples of such salt include those similar to the salts of compound (IA).

Compound (IA) can be produced according to the method described in the following Reaction Scheme 1.

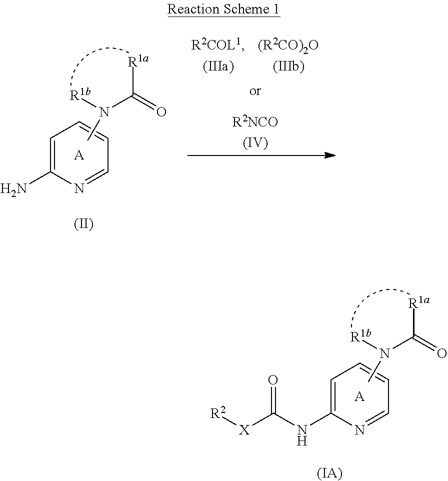

wherein L¹ is a leaving group, and other symbols are as defined above.

Compound (IA) wherein Y═O can be produced by reacting compound (II) with compound (IIIa), compound (IIIb) or compound (IV) in the presence of a base or an acid, if desired.

Compound (IIIa), compound (IIIb) and compound (IV) may be commercially available products, or can also be produced according to a method known per se or a method analogous thereto.

Examples of the “leaving group” for L¹ include a hydroxy group, a halogen atom (e.g., fluorine, chlorine, bromine, iodine), an optionally halogenated C₁₋₅ alkylsulfonyloxy group (e.g., methanesulfonyloxy, ethanesulfonyloxy, trichloromethanesulfonyloxy etc.), an optionally substituted C₆₋₁₀ arylsulfonyloxy group, an optionally substituted phenyloxy group, an optionally substituted benzothiazol-2-ylthio group and the like.

Examples of the “optionally substituted C₆₋₁₀ arylsulfonyloxy group” include a C₆₋₁₀ arylsulfonyloxy (e.g., phenylsulfonyloxy, naphthylsulfonyloxy etc.) optionally having 1 to 3 substituents selected from C₁₋₆ alkyl (e.g., methyl, ethyl, propyl, isopropyl, butyl, isobutyl, sec-butyl, tert-butyl, pentyl, hexyl etc.), C₁₋₆ alkoxy (e.g., methoxy, ethoxy, propoxy, isopropoxy, butoxy, isobutoxy, sec-butoxy, pentyloxy, hexyloxy etc.) and nitro, and the like. Specific examples include benzenesulfonyloxy, m-nitrobenzenesulfonyloxy, p-toluenesulfonyloxy and the like.

Examples of the “optionally substituted phenyloxy group” include a phenyloxy group optionally having 1 to 3 substituents selected from C₁₋₆ alkyl (e.g., methyl, ethyl, propyl, isopropyl, butyl, isobutyl, sec-butyl, tert-butyl, pentyl, hexyl etc.), C₁₋₆ alkoxy (e.g., methoxy, ethoxy, propoxy, isopropoxy, butoxy, isobutoxy, sec-butoxy, pentyloxy, hexyloxy etc.) and nitro, and the like. Specific examples include phenyloxy, 4-nitrophenoxy and the like.

Examples of the “optionally substituted benzothiazol-2-ylthio group” include a benzothiazol-2-ylthio group optionally having 1 to 3 substituents selected from C₁₋₆ alkyl (e.g., methyl, ethyl, propyl, isopropyl, butyl, isobutyl, sec-butyl, tert-butyl, pentyl, hexyl etc.), C₁₋₆ alkoxy (e.g., methoxy, ethoxy, propoxy, isopropoxy, butoxy, isobutoxy, sec-butoxy, pentyloxy, hexyloxy etc.) and nitro, and the like. Specific examples include benzothiazol-2-ylthio and the like.

The amount of compound (IIIa), compound (IIIb) or compound (IV) to be used is about 1 to 10 mol, preferably about 1 to 2 mol, per 1 mol of compound (II).

Examples of the “base” include basic salts such as sodium carbonate, potassium carbonate, cesium carbonate, sodium hydrogen carbonate and the like; aromatic amines such as pyridine, lutidine and the like; tertiary amines such as triethylamine, tripropylamine, tributylamine, cyclohexyldimethylamine, 4-dimethylaminopyridine, N-methylpiperidine, N-methylpyrrolidine, N-methylmorpholine and the like; alkali metal hydrides such as sodium hydride, potassium hydride and the like; metal amides such as sodium amide, lithiumdiisopropylamide, lithiumhexamethyl disilazide and the like; metal alkoxides such as sodium methoxide, sodium ethoxide, potassium tert-butoxide and the like, and the like.

The amount of the “base” to be used is generally about 0.1 to 10 equivalents, preferably 0.8 to 2 equivalents, relative to compound (II).

Examples of the “acid” include methanesulfonic acid, p-toluenesulfonic acid, camphorsulfonic acid and the like.

The amount of “acid” to be used is generally about 0.1 to 10 equivalents, preferably 0.8 to 3 equivalents, relative to compound (II).

This reaction is advantageously carried out without solvent or using a solvent inert to the reaction. The solvent is not particularly limited as long as the reaction proceeds, and preferable examples thereof include water; ethers such as diethyl ether, tetrahydrofuran, 1,4-dioxane, 1,2-dimethoxyethane and the like; hydrocarbons such as benzene, toluene, cyclohexane, hexane and the like hydrocarbon; amides such as N,N-dimethylformamide, N,N-dimethylacetamide and the like; halogenated hydrocarbons such as dichloromethane, chloroform, carbon tetrachloride, 1,2-dichloroethane and the like; nitrites such as acetonitrile, propionitrile and the like; sulfoxides such as dimethylsulfoxide and the like; nitrogen-containing aromatic hydrocarbons such as pyridine, lutidine, quinoline and the like, a mixed solvent thereof and the like.

The reaction temperature is generally about −40 to 150° C., preferably 0 to 100° C.

The reaction time is generally 5 min to 24 hr, preferably 10 min to 5 hr.

Alternatively, compound (II) may be reacted with R²COOH in the presence of a suitable condensation agent.

The amount of R²COOH to be used is generally about 0.8 to about 10 mol, preferably about 0.8 to about 2 mol, per 1 mol of compound (II).

Examples of the “condensation agent” include N,N′-carbodiimides such as N,N′-dicyclohexylcarbodiimide, 1-ethyl-3-(3-dimethylaminopropyl)carbodiimide hydrochloride (WSC) and the like; azolides such as N,N′-carbonylimidazole and the like; 2-halogenopyridinium salts such as 2-chloro-1-methylpyridinium iodide, 2-fluoro-1-methylpyridinium iodide and the like; N-ethoxycarbonyl-2-ethoxy-1,2-dihydroquinoline, diethyl cyanophosphate, phosphorus oxychloride, acetic anhydride and the like.

The amount of “condensation agent” to be used is generally about 0.8 to about 5 mol, preferably about 1 to about 3 mol, per 1 mol of compound (II).

The reaction may also be carried out in the co-presence lo of a base, if desired. Examples of the “base” include basic salts such as potassium acetate, sodium acetate and the like; tertiary amines such as triethylamine, tripropylamine, tributylamine, cyclohexyldimethylamine, 4-dimethylaminopyridine, N-methylpiperidine, N-methylpyrrolidine, N-methylmorpholine and the like, and the like. In addition, the reaction may also be carried out in the co-presence of a condensation promoter such as 1-hydroxy-1H-benzotriazole (HOBt) monohydrate and the like, if desired.

The amount of “base” to be used is generally about 0.5 to about 5 mol, preferably about 2 to about 3 mol, per 1 mol of compound (II).

This reaction is advantageously carried out using a solvent inert to the reaction. Preferable examples of such solvent include alcohols such as methanol, ethanol, propanol and the like; hydrocarbons such as hexane, cyclohexane, benzene, toluene, xylene and the like; ethers such as diethyl ether, diisopropyl ether, tetrahydrofuran, 1,4-dioxane, 1,2-dimethoxyethane and the like; amides such as N,N-dimethylformamide, N,N-dimethylacetamide, hexamethylphosphoric triamide, 1-methylpyrrolidin-2-one and the like; sulfoxides such as dimethylsulfoxide and the like; halogenated hydrocarbons such as dichloromethane, chloroform, carbon tetrachloride, 1,2-dichloroethane and the like; nitrites such as acetonitrile, propionitrile and the like; acid anhydrides such as acetic anhydride and the like, a mixed solvent thereof and the like.

The reaction time is generally about 10 min to about 48 hr, preferably about 30 min to about 24 hr.

The reaction temperature is generally about −20 to about 150° C., preferably about 0 to about 100° C.

This reaction time can be shortened by using a microwave apparatus and the like.

The thus-obtained compound (IA) can be isolated from the reaction mixture according to a conventional method, and can be isolated and purified by a known method such as concentration, concentration under reduced pressure, solvent extraction, crystallization, recrystallization, phase transfer, chromatography and the like.

Compound (IA) wherein X═—NH— and Y═O can also be produced according to the method described in the following Reaction Scheme 2. That is, compound (IA) can be produced by subjecting compound (II) to 2,2,2-trichloroethoxycarbonylation or converting to bis(2,2,2-trichloroethoxycarbonyl) compound using 2,2,2-trichloroethyl chloroformate to give compound (I′), compound (I″A) or a mixture of compound (I′) and compound (I″A), and by reacting the compound with compound (V).

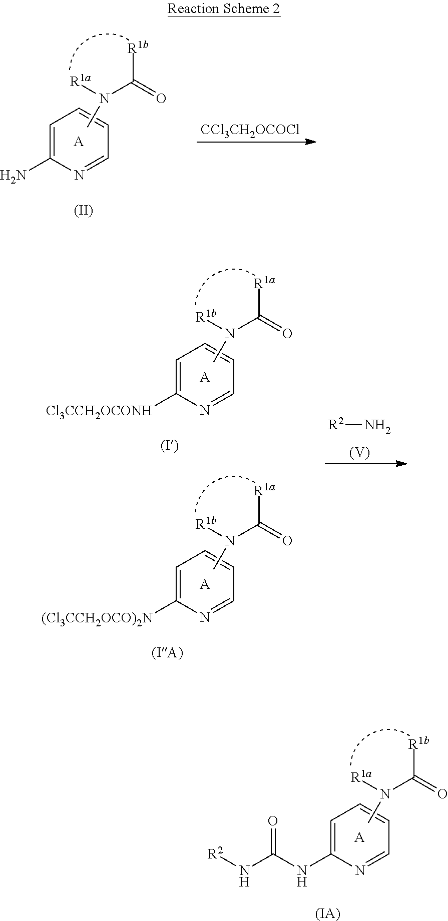

wherein each symbol is as defined above.

Compound (I′), compound (I″A) or a mixture of compound (I′) and compound (I″A) can be produced from compound (II) in the same manner as in the production of compound (IA) from compound (II) described in Reaction Scheme 1.

Compound (IA) can be produced by reacting compound (I′), compound (I″A) or a mixture of compound (I′) and compound (I″A) with compound (V) in a solvent that does not adversely influence the reaction, under basic condition.

Compound (V) may be commercially available product, or can also be produced according to a method known per se or a method analogous thereto.

The amount of compound (V) to be used is generally about 2 to 10 mol, preferably about 2 to 5 mol compound (I′), per 1 mol of compound (I′), compound (I″A) or a mixture of compound (I′) and compound (I″A).

Examples of the “base” include pyridine, triethylamine, diisopropylethylamine, potassium carbonate, sodium carbonate, sodium hydride, potassium hydride and the like.

The amount of “base” to be used is generally about 2 to 10 mol, preferably about 2 to 5 mol, per 1 mol of compound (I′), compound (I″A) or a mixture of compound (I′) and compound (I″A).

Examples of the solvent that does not adversely influence the reaction include ethers such as tetrahydrofuran and the like; halogenated hydrocarbons such as chloroform and the like; aromatic hydrocarbons such as toluene and the like; amides such as N,N-dimethylformamide and the like; sulfoxides such as dimethylsulfoxide and the like, and the like. These bases may be used in a mixture at an appropriate ratio.

The reaction temperature is generally about −50° C. to 200° C.

The reaction time is generally about 0.5 to about 36 hr.

The thus-obtained compound (IA) can be isolated from the reaction mixture according to a conventional method, and can be isolated and purified by a known method such as concentration, concentration under reduced pressure, solvent extraction, crystallization, recrystallization, phase transfer, chromatography and the like.

Compound (II) can be produced according to the method described in the following Reaction Scheme 3.

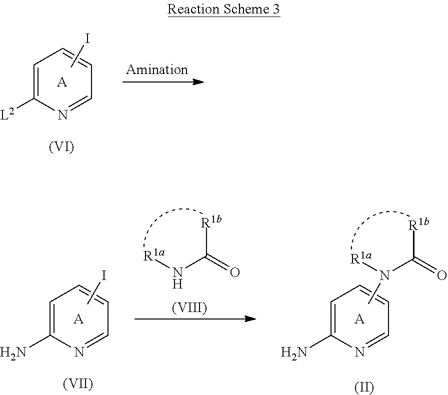

wherein L² is a halogen atom (e.g., fluorine, chlorine, bromine, iodine), and the other symbols are as defined above.

Compound (VII) can be produced by reacting compound (VI) with an amine in the presence of a base, if desired. In addition, a copper catalyst such as copper, copper salt and the like can be used, as necessary. Alternatively, compound (VII) can also be produced according to the Buchwald cross-coupling reaction.

Compound (VI) can be easily commercially available product, or can also be produced according to a method known per se or a method analogous thereto.

Examples of the “amine” include aqueous ammonia, benzylamine, 4-methoxybenzylamine and the like.

The amount of “amine” to be used is generally about 0.8 to about 10 mol, preferably about 1 to about 5 mol, per 1 mol of compound (VI) in the case of the “amine” is benzylamine, 4-methoxybenzylamine or the like. It is generally about 5 to about 200 mol, preferably about 10 to about 100 mol, per 1 mol of compound (VI) in the case of the “amine” is aqueous ammonia.

Examples of the “base” include basic salts such as sodium carbonate, potassium carbonate, cesium carbonate, sodium hydrogen carbonate and the like; aromatic amines such as pyridine, lutidine and the like; tertiary amines such as triethylamine, tripropylamine, tributylamine, cyclohexyldimethylamine, 4-dimethylaminopyridine, N,N-dimethylaniline, N-methylpiperidine, N-methylpyrrolidine, N-methylmorpholine and the like; alkali metal hydrides such as sodium hydride, potassium hydride and the like; metal amides such as sodium amide, lithiumdiisopropylamide, lithiumhexamethyl disilazide and the like; metal alkoxides such as sodium methoxide, sodium ethoxide, sodium tert-butoxide, potassium tert-butoxide and the like, and the like.

The amount of “base” to be used is generally about 0.8 to about 10 mol, preferably about 1 to about 5 mol, per 1 mol of compound (VI).

This reaction is advantageously carried out using a solvent inert to the reaction. The solvent is not particularly limited as long as the reaction proceeds, and preferable lo examples thereof include alcohols such as methanol, ethanol, propanol and the like; ethers such as diethyl ether, tetrahydrofuran, 1,4-dioxane, 1,2-dimethoxyethane and the like; hydrocarbons such as benzene, toluene, cyclohexane, hexane and the like; amides such as N,N-dimethylformamide, N,N-dimethylacetamide and the like; halogenated hydrocarbons such as dichloromethane, chloroform, carbon tetrachloride, 1,2-dichloroethane and the like; nitrites such as acetonitrile, propionitrile and the like; sulfoxides such as dimethylsulfoxide and the like, a mixed solvent thereof and the like.

Examples of the “copper catalyst” include copper, halogenated copper (CuI, CuBr, CuCl etc.), copper oxide (CuO) and the like.

The amount of “copper catalyst” to be used is generally about 0.1 to about 10 mol, preferably about 0.5 to about 2 mol, per 1 mol of compound (VI).

When compound (VII) is synthesized according to the Buchwald reaction, examples of the “palladium catalyst” include palladium acetate, palladium chloride, tetrakis(triphenylphosphine)palladium, bis(dibenzylideneacetone)palladium and the like. The “ligand” is preferably a phosphine, and examples thereof include trialkylphosphine, triarylphosphine, trialkoxyphosphine and the like.

The amount of “palladium catalyst” to be used is generally about 0.001 to about 5 mol, preferably about 0.01 to about 0.5 mol, per 1 mol of compound (VI). The amount of “ligand” to be used is generally about 0.001 to about 10 mol, preferably about 0.01 to about 1 mol, per 1 mol of compound (VI).

The reaction time is generally about 30 min to about 72 hr, preferably about 1 hr to about 48 hr.

The reaction temperature is generally about −20 to about 200° C., preferably about 0 to about 150° C.

This reaction time can be shortened by using a microwave apparatus and the like.

When a protecting group such as benzyl and the like is bond to the amino group, compound (VII) can be produced by removing the protecting group. The removal of the protecting group can be carried out according to a reaction known per se, for example, the reaction by the method described in T.W. Green, Protective Groups in Organic Synthesis, vol. 3, 1999, Chapter of Protection for the Amino Group, and the like.

Compound (VII) can be used for the next reaction directly as the reaction mixture or as a crude product. In addition, it can also be isolated from the reaction mixture according to a conventional method, and can be purified by a known separation means (e.g., recrystallization, distillation, chromatography etc.).

Compound (II) can be produced by subjecting compound (VII) to coupling reaction with compound (VIII).

This reaction is carried out in the presence of a copper catalyst in a solvent under basic condition. Where necessary, a ligand can be used.

The amount of compound (VIII) to be used is generally about 0.5 to about 10 mol, preferably about 0.9 to about 3 mol, per 1 mol of compound (VII).

Compound (VIII) can be easily commercially available product, or can also be produced according to a method known per se or a method analogous thereto.

Examples of the “base” include basic salts such as sodium carbonate, potassium carbonate, cesium carbonate, sodium hydrogen carbonate, potassium phosphate and the like; aromatic amines such as pyridine, lutidine and the like; tertiary amines such as triethylamine, tripropylamine, tributylamine, cyclohexyldimethylamine, 4-dimethylaminopyridine, N,N-dimethylaniline, N-methylpiperidine, N-methylpyrrolidine, N-methylmorpholine and the like; alkali metal hydrides such as sodium hydride, potassium hydride and the like; metal amides such as sodium amide, lithiumdiisopropylamide, lithiumhexamethyl disilazide, potassium hexamethyl disilazide and the like; metal alkoxides such as sodium methoxide, sodium ethoxide, sodium tert-butoxide, potassium tert-butoxide and the like, and the like.

The amount of “base” to be used is generally about 0.8 to about 10 mol, preferably about 1 to about 5 mol, per 1 mol of compound (VII).

This reaction is advantageously carried out using a solvent inert to the reaction. The solvent is not particularly limited as long as the reaction proceeds, and preferable examples thereof include ethers such as diethyl ether, tetrahydrofuran, 1,4-dioxane, 1,2-dimethoxyethane and the like; hydrocarbons such as benzene, toluene, cyclohexane, hexane and the like hydrocarbon; amides such as N,N-dimethylformamide, N,N-dimethylacetamide and the like; halogenated hydrocarbons such as dichloromethane, chloroform, carbon tetrachloride, 1,2-dichloroethane and the like; nitrites such as acetonitrile, propionitrile and the like; sulfoxides such as dimethylsulfoxide and the like, a mixed solvent thereof and the like.

Examples of the “copper catalyst” include copper, halogenated copper (CuI, CuBr, CuCl etc.), copper oxide (CuO), copper sulfate, copper thiocyanate (CuSCN), copper acetate (Cu(OAc)₂) and the like.

The amount of “copper catalyst” to be used is generally about 0.001 to about 10 mol, preferably about 0.01 to about 2 mol, per 1 mol of compound (VII).

Examples of the “ligand” include diamines such as ethylenediamine, N-methylethylenediamine, N,N′-dimethylethylenediamine, cyclohexyldiamine, N-methylcyclohexyldiamine, N,N′-dimethylcyclohexyldiamine and the like; amino acids such as P-alanine, glycine and the like.

The amount of “ligand” to be used is generally about 0.001 to about 10 mol, preferably about 0.01 to about 1 mol, per 1 mol of compound (VII).

The reaction time is generally about 30 min to about 72 hr, preferably about 1 hr to about 60 hr.

The reaction temperature is generally about −20 to about 200° C., preferably about 0 to about 150° C.

This reaction time can be shortened by using a microwave apparatus and the like.

Compound (II) can be used for the next reaction directly as the reaction mixture or as a crude product. In addition, it can also be isolated from the reaction mixture according to a conventional method, and can be purified by a known separation means (e.g., recrystallization, distillation, chromatography etc.).

Compound (IA) can also be produced according to the method described in the following Reaction Scheme 4.

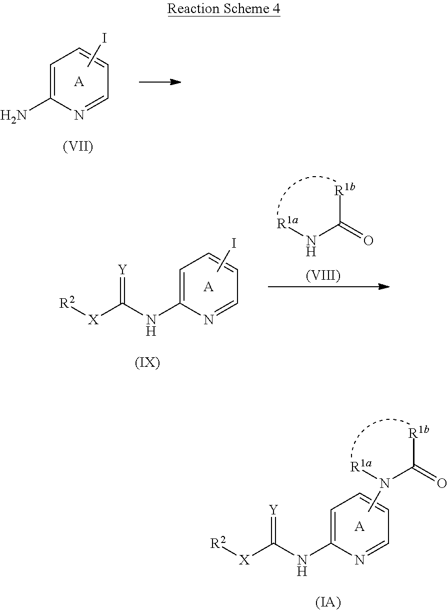

wherein each symbol is as defined above.

Compound (IX) can be produced from compound (VII) in the same manner as in the production of compound (IA) from compound (II) described in Reaction Scheme 1.

Compound (IA) can be produced from compound (IX) in the same manner as in the production of compound (II) from compound (VII) described in Reaction Scheme 3.

The thus-obtained compound (IA) can be isolated from the reaction mixture according to a conventional method, and can be isolated and purified by a known method such as concentration, concentration under reduced pressure, solvent extraction, crystallization, recrystallization, phase transfer, chromatography and the like.

When a substituent that compound (IA) has contains a convertible functional group (e.g., carboxyl group, amino group, hydroxy group, carbonyl group, mercapto group, C₁₋₆ alkoxy-carbonyl group, C₆₋₁₄ aryloxy-carbonyl group, C₇₋₁₆ aralkyloxy-carbonyl group, sulfo group, halogen atom etc.), various compounds, can be produced by converting such functional groups by a method known per se or a method according thereto.

In the case of a carboxyl group, for example, conversion is possible by a reaction such as esterification, reduction, amidation, conversion reaction to an optionally protected amino group, and the like.

In the case of an amino group, for example, conversion is possible by a reaction such as amidation, sulfonylation, nitrosation, alkylation, arylation, imidation and the like.

In the case of a hydroxy group, for example, conversion is possible by a reaction such as esterification, carbamoylation, sulfonylation, alkylation, arylation, oxidation, halogenation and the like.

In the case of a carbonyl group, for example, conversion is possible by a reaction such as reduction, oxidation, imination (including oximation, hydrazonation), (thio)ketalation, alkylidenation, thiocarbonylation and the like.

In the case of a mercapto group, for example, conversion is possible by a reaction such as alkylation, oxidation and the like.

In the case of a C₁₋₆ alkoxy-carbonyl group, a C₆₋₁₄ aryloxy-carbonyl group or a C₇₋₁₆ aralkyloxy-carbonyl group, for example, conversion is possible by a reaction such as reduction, hydrolysis and the like.

In the case of a sulfo group, for example, conversion is possible by a reaction such as sulfonamidation, reduction and the like.

In the case of a halogen atom, for example, conversion is possible by a reaction such as various nucleophilic substitution reactions, various coupling reactions and the like.

In each of the aforementioned reactions, when the compound is obtained in a free form, it may be converted to a salt according to a conventional method, and when the compound is obtained as a salt, it can also be converted to a free form or other salt according to a conventional method.

In each reaction of the aforementioned production methods of compound (IA) and each reaction of starting material compound syntheses, when a starting material compound has an amino group, a carboxyl group or a hydroxy group as a substituent, a protecting group generally used in peptide chemistry and the like may be introduced into these groups. By removal of the protecting group as necessary after the reaction, the objective compound can be obtained.

As the amino-protecting group, for example, formyl group; C₁₋₆ alkyl-carbonyl group (e.g., acetyl, ethylcarbonyl etc.), phenylcarbonyl group, C₁₋₆ alkoxy-carbonyl group (e.g., methoxycarbonyl, ethoxycarbonyl, tert-butoxycarbonyl (Boc) etc.), allyloxycarbonyl (Alloc) group, phenyloxycarbonyl group, fluorenylmethoxycarbonyl (Fmoc) group, C₇₋₁₀ aralkyl-carbonyl group (e.g., benzylcarbonyl etc.), C₇₋₁₀ aralkyl-oxycarbonyl group (e.g., benzyloxycarbonyl(Z) etc.), C₇₋₁₀ aralkyl group (e.g., benzyl etc.), trityl group, phthaloyl group, N,N-dimethylaminomethylene group etc., each optionally having substituent(s), and the like can be used. As these substituents, phenyl group, halogen atom (e.g., fluorine, chlorine, bromine, iodine etc.), C₁₋₆ alkyl-carbonyl group (e.g., methylcarbonyl, ethylcarbonyl, butylcarbonyl etc.), nitro group etc. can be used. The number of the substituent(s) is about 1 to 3.

As the carboxyl-protecting group, for example, C₁₋₆ alkyl group (e.g., methyl, ethyl, n-propyl, isopropyl, n-butyl, tert-butyl etc.), allyl group, benzyl group, phenyl group, trityl group, trialkylsilyl group, each optionally having substituent(s), and the like can be used. As these substituents, halogen atom (e.g., fluorine, chlorine, bromine, iodine etc.), formyl group, C₁₋₆ alkyl-carbonyl group (e.g., acetyl, ethylcarbonyl, butylcarbonyl etc.), nitro group and the like can be used. The number of the substituent(s) is about 1 to 3.

As the hydroxyl-protecting group, for example, C₁₋₆ alkyl group (e.g., methyl, ethyl, n-propyl, isopropyl, n-butyl, tert-butyl etc.), C₇₋₁₀ aralkyl group (e.g., benzyl etc.), formyl group, C₁₋₆ alkyl-carbonyl group (e.g., acetyl, ethylcarbonyl etc.), benzoyl group, C₇₋₁₀ aralkyl-carbonyl group (e.g., benzylcarbonyl etc.), tetrahydropyranyl group, furanyl group, silyl group, each optionally having substituent(s), and the like can be used. As these substituents, halogen atom (e.g., fluorine, chlorine, bromine, iodine etc.), C₁₋₆ alkyl group (e.g., methyl, ethyl, n-propyl etc.), phenyl group, C₇₋₁₀ aralkyl group (e.g., benzyl etc.), C₁₋₆ alkoxy group (e.g., methoxy, ethoxy, n-propoxy etc.), nitro group and the like can be used. The number of the substituent(s) is about 1 to 4.

The thus-obtained compound (IA) can be isolated and purified by a known means, for example, solvent extraction, liquid conversion, phase transfer, crystallization, recrystallization, chromatography and the like.

Each starting material compound used for the production of compound (IA) can also be isolated and purified by a known means such as those mentioned above and the like. It may also be used as a starting material in the form of a reaction mixture in the next step without isolation.

The solvent to be used for the above-mentioned recrystallization may be, for example, water, alcohols, ethers, hydrocarbons, amides, halogenated hydrocarbons, nitriles, ketones, esters, sulfoxides, organic acids and the like. These solvents may be used alone, or two or more kinds of solvents may be mixed at a suitable ratio, for example, 1:1-1:10, and used.

When compound (IA) is present as a configurational isomer (stereoisomer), diastereomer, conformer or the like, they can be respectively isolated by a known means. When compound (IA) is an optically active form, a racemate can be separated into a (+) form and a (−) form by a general optical resolution means.

When compound (IA) contains optical isomer, stereoisomer, positional isomer or rotamer, each of these can also be contained as compound (IA), as well as can be obtained as a single product by a synthesis method and a separation method known per se.

For example, the method of optical resolution may be a method known per se, such as a fractional recrystallization method, a chiral column method, a diastereomer method, etc.

### 1) Fractional Recrystallization Method

A method wherein a salt of a racemate with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine, etc.) is formed, which is separated by a fractional recrystallization method, and if desired, a free optical isomer is obtained by a neutralization step.

### 2) Chiral Column Method

A method wherein a racemate or a salt thereof is applied to a column for separation of an optical isomer (a chiral column) to allow separation. In the case of a liquid chromatography, for example, a mixture of the optical isomers is applied to a chiral column such as ENALTIO-OVM (manufactured by Tosoh Corporation), CHIRAL series (manufactured by Daicel Chemical Industries, Ltd.) and the like, and developed with water, various buffers (e.g., phosphate buffer) and organic solvents (e.g., ethanol, methanol, isopropanol, acetonitrile, trifluoroacetic acid, diethylamine) solely or in admixture to separate the optical isomer. In the case of a gas chromatography, for example, a chiral column such as CP-Chirasil-DeX CB (manufactured by GL Sciences Inc.) and the like is used to allow separation.

### 3) Diastereomer Method

A method wherein a racemic mixture is prepared into a diastereomeric mixture by chemical reaction with an optically active reagent, which is made into a single substance by a typical separation means (e.g., a fractional recrystallization method, a chromatography method, etc.) and the like, and is subjected to a chemical treatment such as hydrolysis and the like to separate an optically active reagent moiety, whereby an optical isomer is obtained. For example, when compound (IA) contains hydroxy group, or primary or secondary amino group in a molecule, the compound and an optically active organic acid (e.g., MTPA [α-methoxy-α-(trifluoromethyl)phenylacetic acid], (−)-menthoxyacetic acid, etc.) and the like are subjected to condensation reaction to give diastereomers in the ester form or in the amide form, respectively. When compound (IA) has a carboxyl group, this compound and an optically active amine or alcohol reagent are subjected to condensation reaction to give diastereomers in the amide form or in the ester form, respectively. The separated diastereomer is converted to an optical isomer of the original compound by acid hydrolysis or base hydrolysis.

A salt of compound (IA) can be produced by a method known per se. For example, when compound (IA) is a basic compound, it can be produced by adding an inorganic acid or organic acid, or when compound (IA) is an acidic compound, by adding an organic base or inorganic base.

Compound (IA) may be a hydrate, and both hydrate and non-hydrate are encompassed in the scope of the present invention. Compound (IA) may be labeled with an isotope (e.g., ³H, ¹⁴C, ³⁵S, ¹²⁵I and the like) or the like.

Since the GSK-3 inhibitor of the present invention selectively inhibits GSK-3 and shows low toxicity and a fewer side effects, it is useful as a safe pharmaceutical product. The GSK-3 inhibitor of the present invention shows a superior GSK-3 selective inhibitory action for a mammal (e.g., mouse, rat, hamster, rabbit, cat, dog, bovine, sheep, monkey, human etc.) and is superior in (oral) absorbability, (metabolic) stability and the like. Therefore, it can be used as an agent for the prophylaxis or treatment of GSK-3 related pathology or diseases, for example, metabolic diseases (e.g., diabetes (type 1 diabetes, type 2 diabetes, gestational diabetes etc.), impaired glucose tolerance, obesity, diabetic neuropathy, diabetic retinopathy, diabetic nephropathy, lipid metabolism abnormalities (hypertriglyceridemia, hypercholesterolemia, hypoHDL-emia, postprandial hyperlipemia etc.) and the like), circulatory diseases (e.g., hypertension, cardiac hypertrophy, angina pectoris, arteriosclerosis and the like), inflammatory diseases (e.g., allergy, asthma, rheumatism, sepsis, psoriasis, colitis, Crohn's disease, COPD etc.), osteoarthritis, liver cirrhosis, alcoholic hepatitis, osteoporosis, cancer and alopecia, and an agent for preventing the progress from impaired glucose tolerance to diabetes.

In the area of neurological diseases, the GSK-3 inhibitor has a neural stem cell differentiation-promoting action. Accordingly, the GSK-3 inhibitor can be used as an agent for the prophylaxis or treatment of neurodegenerative diseases such as Alzheimer's disease, mild cognitive impairment (MCI), Huntington's chorea, Parkinson's disease, epilepsy, amyotrophic lateral sclerosis (ALS), multiple sclerosis, cerebellum spinal cord denaturation, Pick disease, peripheral nerve disorders and the like and mental diseases such as schizophrenia, depression, anxiety, bipolar disorder, PTSD (posttraumatic stress disorder; hereinafter sometimes to be abbreviated to PTSD) and the like. Based on cell protection action and/or function regeneration action, it can be used as an agent for the prophylaxis or treatment of ischemic diseases such as cerebral infarction, myocardial infarction and the like. Particularly preferred is an agent for the prophylaxis or treatment of diabetes or neurodegenerative disease.

For diagnostic criteria of diabetes, Japan Diabetes Society reported new diagnostic criteria in 1999.

According to this report, diabetes is a condition showing any of a fasting blood glucose level (glucose concentration of intravenous plasma) of not less than 126 mg/dl, a 75 g oral glucose tolerance test (75 g OGTT) 2 h level (glucose concentration of intravenous plasma) of not less than 200 mg/dl, and a non-fasting blood glucose level (glucose concentration of intravenous plasma) of not less than 200 mg/dl. A condition not falling under the above-mentioned diabetes and different from “a condition showing a fasting blood glucose level (glucose concentration of intravenous plasma) of less than 110 mg/dl or a 75 g oral glucose tolerance test (75 g OGTT) 2 h level (glucose concentration of intravenous plasma) of less than 140 mg/dl” (normal type) is called a “borderline type”.

In addition, ADA (American Diabetes Association) reported new diagnostic criteria of diabetes in 1997 and WHO in 1998.

According to these reports, diabetes is a condition showing a fasting blood glucose level (glucose concentration of intravenous plasma) of not less than 126 mg/dl and a 75 g oral glucose tolerance test 2 h level (glucose concentration of intravenous plasma) of not less than 200 mg/dl.

According to the above-mentioned reports, impaired glucose tolerance is a condition showing a fasting blood glucose level (glucose concentration of intravenous plasma) of less than 126 mg/dl and a 75 g oral glucose tolerance test 2 h level (glucose concentration of intravenous plasma) of not less than 140 mg/dl and less than 200 mg/dl. According to the report of ADA, a condition showing a fasting blood glucose level (glucose concentration of intravenous plasma) of not less than 110 mg/dl and less than 126 mg/dl is called IFG (Impaired Fasting Glucose). According to the report of WHO, among the IFG (Impaired Fasting Glucose), a condition showing a 75 g oral glucose tolerance test 2 h level (glucose concentration of intravenous plasma) of less than 140 mg/dl is called IFG (Impaired Fasting Glycemia).

Compound (IA) of the present invention can be also used as an agent for the prophylaxis or treatment of diabetes, borderline type, impaired glucose tolerance, IFG (Impaired Fasting Glucose) and IFG (Impaired Fasting Glycemia), as determined according to the above-mentioned new diagnostic criteria. Moreover, the compound of the present invention can prevent progress of borderline type, impaired glucose tolerance, IFG (Impaired Fasting Glucose) or IFG (Impaired Fasting Glycemia) into diabetes.

When compound (IA) of the present invention is applied to each of the above-mentioned diseases, it can be used in an appropriate combination with a pharmaceutical agent or a treatment method generally employed for the disease. For example, acetylcholine esterase inhibitors (e.g., donepezil, rivastigmine, galanthamine, zanapezil (TAK-147) etc.), antidementian agents (memantine etc.), inhibitors of β amyloid protein production, secretion, accumulation, coagulation and/or deposition, β secretase inhibitors (e.g., 6-(4-biphenylyl)methoxy-2-[2-(N,N-dimethylamino)ethyl]tetralin, 6-(4-biphenylyl)methoxy-2-(N,N-dimethylamino)methyltetralin, 6-(4-biphenylyl)methoxy-2-(N,N-dipropylamino)methyltetralin, 2-(N,N-dimethylamino)methyl-6-(4′-methoxybiphenyl-4-yl)methoxytetralin, 6-(4-biphenylyl)methoxy-2-[2-(N,N-diethylamino)ethyl]tetralin, 2-[2-(N,N-dimethylamino)ethyl]-6-(4′-methylbiphenyl-4-yl)methoxytetralin, 2-[2-(N,N-dimethylamino)ethyl]-6-(4′-methoxybiphenyl-4-yl)methoxytetralin, 6-(2′,4′-dimethoxybiphenyl-4-yl)methoxy-2-[2-(N,N-dimethylamino)ethyl]tetralin, 6-[4-(1,3-benzodioxol-5-yl)phenyl]methoxy-2-[2-(N,N-dimethylamino)ethyl]tetralin, 6-(3′,4′-dimethoxybiphenyl-4-yl)methoxy-2-[2-(N,N-dimethylamino)ethyl]tetralin, an optically active form thereof, a salt thereof and a hydrate thereof, OM99-2 (WO01/00663)), γ secretase inhibitory agent, β amyloid protein coagulation inhibitory agent (e.g., PTI-00703, ALZHEMED (NC-531), PPI-368 (JP-A-11-514333), PPI-558 (JP-A-2001-500852), SKF-74652 (Biochem. J. (1999), 340(1), 283-289)), β amyloid vaccine, β amyloid degrading enzyme and the like, cerebral function activators (e.g., aniracetam, nicergoline etc.), other therapeutic drug for Parkinson's disease [(e.g., dopamine receptor agonists (L-DOPA, bromocriptine, pergolide, talipexole, pramipexole, Cabergoline, adamantadine etc.), a monoamine oxidase (MAO) inhibitors (deprenyl, Selgiline (selegiline), remacemide, riluzole etc.), anticholinergic agents (e.g., trihexyphenidyl, biperiden etc.), COMT inhibitors (e.g., entacapone etc.)], therapeutic drug for amyotropic lateral sclerosis (e.g., riluzole etc., neurotrophic factor etc.), therapeutic drug for abnormal behavior, wandering and the like due to the progress of dementia (e.g., sedative drug, antianxiety drug etc.), apoptosis inhibitors (e.g., CPI-1189, IDN-6556, CEP-1347 etc.), neuronal differentiation or regeneration promoters (e.g., leteprinim, xaliproden (SR-57746-A), SB-216763, Y-128, VX-853, prosaptide, 5,6-dimethoxy-2-[2,2,4,6,7-pentamethyl-3-(4-methylphenyl)-2,3-dihydro-1-benzofuran-5-yl]isoindoline, 5,6-dimethoxy-2-[3-(4-isopropylphenyl)-2,2,4,6,7-pentamethyl-2,3-dihydro-1-benzofuran-5-yl]isoindoline, 6-[3-(4-isopropylphenyl)-2,2,4,6,7-pentamethyl-2,3-dihydro-1-benzofuran-5-yl]-6,7-dihydro-5H-[1,3]dioxolo[4,5-f]isoindole and optically active forms, salts and hydrates, etc. thereof), antidepressants (e.g., desipramine, amitriptyline, imipramine, tramadol etc.), anticonvulsants (e.g., lamotrigine etc.), antianxiety drugs (e.g., benzodiazepine etc.), non-steroidal anti-inflammatory drugs (e.g., meloxicam, tenoxicam, indomethacin, ibuprofen, celecoxib, rofecoxib, aspirin, indomethacin etc.), disease-modifying anti-rheumatic drugs (DMARDs), anti-cytokine drugs (TNF inhibitor, MAP kinase inhibitor and the like), steroidal drugs (e.g., dexamethasone, hexestrol, cortisone acetate etc.), therapeutic agents for incontinence or frequent urination (e.g., flavoxate hydrochloride, oxybutynin hydrochloride, propiverine hydrochloride etc.), phosphodiesterase inhibitors (e.g., sildenafil (citrate) etc.), dopamine agonists (e.g., apomorphine etc.), antiarrhythmics (e.g., mexiletine etc.), sex hormones or derivatives thereof (e.g., progesterone, estradiol, estradiol benzoate etc.), therapeutic agents for osteoporosis (e.g., alfacalcidol, calcitriol, elcatonin, calcitonin salmon, estriol, ipriflavone, disodium pamidronate, sodium alendronate hydrate, disodium incadronate etc.), parathyroid hormone (PTH), calcium receptor antagonists and the like. Particularly, a combined use with a β secretase inhibitory agent such as 6-(4-biphenylyl)methoxy-2-[2-(N,N-dimethylamino)ethyl]tetralin hydrochloride-monohydrate etc., and the like is preferable.

In addition, a combined use with a transplantation method of neural stem cell or neural precursor cell, or fetal neural tissue prepared from embryonic stem cell or nervous tissue, and a combined use with a pharmaceutical agent such as an immunosuppressant after the transplantation and the like.

Examples of therapeutic agent for diabetes include insulin preparations (e.g., animal insulin preparation extracted from the pancreas of bovine, swine; human insulin preparation genetically synthesized using Escherichia coli, yeast; zinc insulin; protamine zinc insulin; insulin fragment or derivatives (e.g., INS-1 etc.) and the like), insulin sensitizers [e.g., pioglitazone hydrochloride, troglitazone, rosiglitazone or maleate thereof, GI-262570, JTT-501, MCC-555, YM-440, KRP-297, CS-011, FK-614, compounds described in WO99/58510 (e.g., (E)-4-[4-(5-methyl-2-phenyl-4-oxazolylmethoxy)benzyloxyimino]-4-phenylbutyric acid), NN-622, AZ-242, BMS-298585, ONO-5816, LM-4156, BM-13-1258, MBX-102, GW-1536 etc.], α-glucosidase inhibitors (e.g., voglibose, acarbose, miglitol, emiglitate etc.), biguanides (e.g., phenformin, metformin, buformin etc.), insulin secretagogues [sulfonylurea (e.g., tolbutamide, glibenclamide, gliclazide, chlorpropamide, tolazamide, acetohexamide, glyclopyramide, glimepiride, glipizide, glybuzole etc.), repaglinide, nateglinide, mitiglinide or calcium salt hydrate thereof, GLP-1 etc.], dipeptidyl-peptidase IV inhibitors (e.g., NVP-DPP-278, PT-100, NVP-DPP-728, LAF237 etc.), β3 agonists (e.g., CL-316243, SR-58611-A, UL-TG-307, SB-226552, AJ-9677, BMS-196085, AZ-40140 etc.), amylin agonists (e.g., pramlintide etc.), phosphotyrosine phosphatase inhibitors (e.g., vanadic acid etc.), gluconeogenesis inhibitors (e.g., glycogen phosphorylase inhibitor, glucose-6-phosphatase inhibitor, glucagon antagonist etc.), SGLUT (sodium-glucose cotransporter) inhibitors (e.g., T-1095 etc.) and the like.

Examples of the therapeutic agents for diabetic complications include aldose reductase inhibitors (e.g., tolrestat, epalrestat, zenarestat, zopolrestat, minalrestat, fidarestat (SNK-860), CT-112 etc.), neurotrophic factors (e.g., NGF, NT-3, BDNF etc.), neurotrophic factor production-secretion promoters [e.g., neurotrophin production-secretion promoters described in WO01/14372 (e.g., 4-(4-chlorophenyl)-2-(2-methyl-1-imidazolyl)-5-[3-(2-methylphenoxy)propyl]oxazole and the like)], PKC inhibitors (e.g., LY-333531 etc.), AGE inhibitors (e.g., ALT946, pimagedine, pyratoxanthine, N-phenacylthiazolium bromide (ALT766), EXO-226 etc.), active oxygen scavengers (e.g., thioctic acid etc.), and cerebral vasodilators (e.g., tiapuride, mexiletine etc.).

Examples of the therapeutic agents for hyperlipidemia include HMG-CoA reductase inhibitors (e.g., pravastatin, simvastatin, lovastatin, atorvastatin, fluvastatin, lipantil, cerivastatin, itavastatin, ZD-4522 or a salt thereof (e.g., sodium salt etc.) and the like), fibrate compounds (e.g., bezafibrate, beclobrate, binifibrate, ciprofibrate, clinofibrate, clofibrate, clofibric acid, etofibrate, fenofibrate, gemfibrozil, nicofibrate, pirifibrate, ronifibrate, simfibrate, theofibrate and the like), squalene synthase inhibitors (e.g., compounds described in WO97/10224, for example, N-[[(3R,5S)-1-(3-acetoxy-2,2-dimethylpropyl)-7-chloro-5-(2,3-dimethoxyphenyl)-2-oxo-1,2,3,5-tetrahydro-4,1-benzoxazepin-3-yl]acetyl]piperidine-4-acetic acid and the like), ACAT inhibitors (e.g., avasimibe, eflucimibe and the like), anion exchange resins (e.g., colestyramine and the like), probucol, nicotinic acid drugs (e.g., nicomol, niceritrol and the like), ethyl icosapentate, phytosterol (e.g., soysterol, γ oryzanol and the like) and the like.

Examples of the antihypertensive agent include angiotensin converting enzyme inhibitors (e.g., captopril, enalapril, delapril etc.), angiotensin II antagonist (e.g., candesartan cilexetil, losartan, eprosartan, valsartan, telmisartan, irbesartan, tasosartan etc.), calcium antagonists (e.g., manidipine, nifedipine, nicardipine, amlodipine, efonidipine etc.), potassium channel openers (e.g., levcromakalim, L-27152, AL 0671, NIP-121 and the like), clonidine and the like.

Examples of the antiobesity agents include antiobesity agents acting on the central nervous system (e.g., dexfenfluramine, fenfluramine, phentermine, sibutramine, anfepramone, dexamphetamine, mazindol, phenylpropanolamine, clobenzorex etc.), pancreatic lipase inhibitors (e.g., orlistat etc.), β3 agonists (e.g., CL-316243, SR-58611-A, UL-TG-307, SB-226552, AJ-9677, BMS-196085, AZ-40140 etc.), anorectic peptides (e.g., leptin, CNTF (ciliary neurotrophic factor) etc.), cholecystokinin agonists (e.g., lintitript, FPL-15849 etc.) and the like.

Examples of the diuretics include xanthine derivatives (e.g., sodium salicylate and theobromine, calcium salicylate and theobromine etc.), thiazide preparations (e.g., ethiazide, cyclopenthiazide, trichloromethiazide, hydrochlorothiazide, hydroflumethiazide, benzylhydrochlorothiazide, penflutizide, polythiazide, methyclothiazide etc.), antialdosterone preparations (e.g., spironolactone, triamterene etc.), carbonate dehydratase inhibitors (e.g., acetazolamide and the like), chlorobenzenesulfonamide preparations (e.g., chlortalidone, mefruside, indapamide etc.), azosemide, isosorbide, etacrynic acid, piretanide, bumetanide, furosemide and the like.

Examples of the chemotherapeutic agents include alkylating agents (e.g., cyclophosphamide, ifosfamide etc.), metabolic antagonists (e.g., methotrexate, 5-fluorouracil or derivative thereof etc.), antitumor antibiotics (e.g., mitomycin, adriamycin etc.), plant-derived antitumor agents (e.g., vincristine, vindesine, Taxol etc.), cisplatin, carboplatin, etoposide and the like. Of these, Furtulon and NeoFurtulon, which are 5-fluorouracil derivatives, and the like are preferable.

Examples of the immunotherapeutic agents include microorganism or bacterial components (e.g., muramyl dipeptide derivative, Picibanil etc.), polysaccharides having immunity potentiating activity (e.g., lentinan, schizophyllan, krestin etc.), cytokines obtained by genetic engineering techniques (e.g., interferon, interleukin (IL) etc.), colony stimulating factors (e.g., granulocyte colony stimulating factor, erythropoietin etc.) and the like, with preference given to interleukins such as IL-1, IL-2, IL-12 and the like.

Examples of the antithrombotic agents include heparin (e.g., heparin sodium, heparin calcium, dalteparin sodium etc.), warfarin (e.g., warfarin potassium etc.), anti-thrombin drugs (e.g., aragatroban etc.), thrombolytic agents (e.g., urokinase, tisokinase, alteplase, nateplase, monteplase, pamiteplase etc.), platelet aggregation inhibitors (e.g., ticlopidine hydrochloride, cilostazol, ethyl icosapentate, beraprost sodium, sarpogrelate hydrochloride etc.) and the like.

Examples of the cachexia improving pharmaceutical agent include cyclooxygenase inhibitors (e.g., indomethacin etc.) [Cancer Research, Vol. 49, pages 5935-5939, 1989], progesterone derivatives (e.g., megestrol acetate) [Journal of Clinical Oncology, Vol. 12, pages 213-225, 1994], glucosteroids (e.g., dexamethasone etc.), metoclopramide agents, tetrahydrocannabinol agents (publications are all as mentioned above), fat metabolism improving agents (e.g., eicosapentanoic acid etc.) [British Journal of Cancer, Vol. 68, pages 314-318, 1993], growth hormones, IGF-1, or antibodies to a cachexia-inducing factor such as TNF-α, LIF, IL-6, oncostatin M and the like.

It is also possible to apply compound (IA) of the present invention to each of the above-mentioned diseases in combination with a biologic (e.g., antibody, vaccine preparation and the like), or as a combination therapy in combination with gene therapy method and the like. Examples of the antibody and vaccine preparation include vaccine preparation to angiotensin II, vaccine preparation to CETP, anti-CETP antibody, anti-TNFα antibody and antibody to other cytokine, amyloid β vaccine preparation, type 1 diabetes vaccine (DIAPEP-277 manufactured by Peptor Ltd. and the like), anti-HIV antibody, HIV vaccine preparation and the like, antibody or vaccine preparation to cytokine, renin-angiotensin enzyme and a product thereof, antibody or vaccine preparation to enzyme or protein involved in blood lipid metabolism, antibody or vaccine to enzyme or protein involved in blood coagulation or fibrinolytic system, antibody or vaccine preparation to protein involved in saccharometabolism or insulin resistance and the like. In addition, a combined use with a biological preparation involved in a growth factor such as GH, IGF and the like is possible. Examples of the gene therapy method include a treatment method using a gene relating to cytokine, renin-angiotensin enzyme and a product thereof, G protein, G protein conjugated receptor and its phosphorylation enzyme, a treatment method using a DNA decoy such as NFκB decoy and the like, a treatment method using an antisense, a treatment method using a gene relating to an enzyme or protein involved in blood lipid metabolism (e.g., gene relating to metabolism, excretion or absorption of cholesterol or triglyceride or HDL-cholesterol or blood phospholipid and the like), a treatment method using a gene relating to an enzyme or protein involved in angiogenesis therapy targeting obstruction of peripheral vessel and the like (e.g., growth factors such as HGF, VEGF etc., and the like), a treatment method using a gene relating to a protein involved in saccharometabolism or insulin resistance, an antisense to cytokine such as TNF and the like, and the like. In addition, it is possible to use compound A in combination with various organ regeneration methods such as heart regeneration, kidney regeneration, pancreas regeneration, blood vessel regeneration and the like or cell transplantation therapy utilizing bone marrow cell (myelomonocytic cell, myeloid stem cell and the like) or an artificial organ utilizing tissue engineering (artificial blood vessel and cardiac muscle cell sheet).

Compound (IA) of the present invention or a salt thereof can be administered orally or parenterally as it is or after mixing with a pharmacologically acceptable carrier. As pharmacologically acceptable carriers, various organic or inorganic carrier substances conventionally used as preparation materials can be used, and added as excipient, lubricant, binder and disintegrant for solid preparations; or solvent, solubilizing agents, suspending agent, isotonicity agent, buffer, soothing agent and the like for liquid preparation. Where necessary, preparation additive such as preservative, antioxidant, colorant, sweetening agent and the like can be used.

For the pharmaceutical agent of the present invention containing compound (IA) or a salt thereof, the dosage form for oral administration is, for example, tablet (including sugar-coated tablet, film-coated tablet), pill, granule, powder, capsule (including soft capsule, microcapsule), syrup, emulsion, suspension and the like, and the dosage form for parenteral administration is, for example, injection, injecting agent, drip infusion, suppository and the like. In addition, it is effective to make a sustained release preparation by combining with a suitable base (e.g., polymer of butyric acid, polymer of glycolic acid, copolymer of butyric acid-glycolic acid, a mixture of polymer of butyric acid and polymer of glycolic acid, polyglycerol fatty acid ester etc.).

While the content of compound (IA) or a salt thereof in the pharmaceutical agent of the present invention varies depending on the form of the pharmaceutical preparation, it is generally about 2 to 85 wt %, preferably about 5 to 70 wt %, relative to the whole preparation.

As a method for forming compound (IA) or a salt thereof in the above-mentioned dosage form, a known production method generally used in the pertinent field can be applied. When the above-mentioned dosage form is produced, various preparation additives such as carrier (e.g., excipient, binder, disintegrant, lubricant and the like), sweetening agent, surfactant, suspending agent, emulsifier and the like generally used in the field of preparation are appropriately added in suitable amounts as necessary for production.

When the compound (IA) or a salt thereof is prepared in to a tablet, for example, it can be produced by adding an excipient, a binder, a disintegrant, a lubricant and the like, and when a pill and a granule are to be prepared, they can be produced by adding an excipient, a binder, a disintegrant and the like. When a powder and a capsule are to be prepared, they can be produced by adding an excipient and the like, and when a syrup is to be prepared, it can be produced by adding a sweetener and the like, and when an emulsion or a suspension is to be prepared, it can be produced by adding a suspending agent, a surfactant, an emulsifier and the like.

Examples of the excipient include lactose, sucrose, glucose, starch, sucrose, crystalline cellulose, powdered glycyrrhiza, mannitol, sodium hydrogen carbonate, calcium phosphate, calcium sulfate and the like.

Examples of the binder include 5-10 wt % starch liquid paste, 10-20 wt % gum arabic solution or gelatin solution, 1-5 wt % tragacanth solution, carboxymethyl cellulose solution, sodium alginate solution, glycerin and the like.

Examples of the disintegrant include starch, calcium carbonate and the like.

Examples of the lubricant include magnesium stearate, stearic acid, calcium stearate, purified talc and the like.

Examples of the sweetener include glucose, fructose, invert sugar, sorbitol, xylitol, glycerin, simple syrup and the like.

Examples of the surfactant include sodium lauryl sulfate, polysorbate 80, sorbitan monofatty acid ester, polyoxyl 40 stearate and the like.

Examples of the suspending agent include gum arabic, sodium alginate, sodium carboxymethyl cellulose, methyl cellulose, bentonite and the like.

Examples of the emulsifier include gum arabic, tragacanth, gelatin, polysorbate 80 and the like.

Furthermore, when the compound (IA) or a salt thereof is produced in the above-mentioned dosage form, a suitable amount of a colorant, a preservative, an aromatic, a corrigent, a stabilizer, viscous agents and the like typically used in the field of preparation can be added on demand.

The pharmaceutical agent of the present invention containing compound (IA) or a salt thereof is stable and low toxic, and can be used safely. The daily dose varies depending on the condition and body weight of patients, the kind of compound, administration route and the like. For example, in the case of oral administration to patients with diabetes, neurodegenerative disease and the like, the daily dose to an adult (body weight about 60 kg) is about 1 to 1000 mg, preferably about 3 to 300 mg, more preferably about 10 to 200 mg, as an active ingredient (the compound (IA) or a salt thereof), which can be given in a single administration or administered in 2 or 3 portions a day.

When the compound (IA) of the present invention or a salt thereof is administered parenterally, it is generally administered in the form of a liquid (e.g., injection). While the dose varies depending on the subject of administration, target organ, symptom, administration method and the like, it is, for example, about 0.01 mg-about 100 mg, preferably about 0.01-about 50 mg, more preferably about 0.01-about 20 mg, in the form of an injection, relative to 1 kg of body weight, which is preferably given by intravenous injection. As the injection, intravenous injection as well as subcutaneous injection, intracutaneous injection, intramuscular injection, instillation and the like are mentioned, and as a sustained release preparation, iontophoresis transdermal agent and the like are mentioned. Such injections are prepared by methods known per se, i.e., by dissolving, suspending or emulsifying the compound (IA) of the present invention or a salt thereof in a sterilized aqueous solution or oily liquid. As an aqueous solution for injection, physiological saline, isotonic solutions containing glucose or other auxiliary drugs (e.g., D-sorbitol, D-mannitol, sodium chloride and the like) and the like, and they can be used in combination with suitable solubilizing agents, such as alcohols (e.g., ethanol), polyalcohols (e.g., propylene glycol, polyethylene glycol), nonionic surfactants (e.g., polysorbate 80, HCO-50) and the like. As an oily liquid, sesame oil, soybean oil and the like, which may be used in combination with solubilizing agents (e.g., benzyl benzoate, benzyl alcohol and the like) and the like. In addition, buffers (e.g., phosphate buffer, sodium acetate buffer), soothing agents (e.g., benzalkonium chloride, procaine hydrochloride and the like), stabilizers (e.g., human serum albumin, polyethylene glycol and the like), preservatives (e.g., benzyl alcohol, phenol and the like) and the like may be added. A prepared injection is generally filled in an ampoule.

When the compound of the present invention is used in combination with other pharmaceutical agent, the administration mode of the compound of the present invention and a combination drug is not particularly limited, and the compound of the present invention and a combination drug only need to be combined on administration. Examples of such administration mode include the following:
(1) administration of a single preparation obtained by simultaneously processing the compound of the present invention and the concomitant drug, (2) simultaneous administration of two kinds of preparations of the compound of the present invention and the concomitant drug, which have been separately produced, by the same administration route, (3) administration of two kinds of preparations of the compound of the present invention and the concomitant drug, which have been separately produced, by the same administration route in a staggered manner, (4) simultaneous administration of two kinds of preparations of the compound of the present invention and the concomitant drug, which have been separately produced, by different administration routes, (5) administration of two kinds of preparations of the compound of the present invention and the concomitant drug, which have been separately produced, by different administration routes in a staggered manner (e.g., administration in the order of the compound of the present invention and the concomitant drug, or in the reverse order) and the like. The dose of the concomitant drug can be appropriately determined based on the dose employed in clinical situations. The mixing ratio of the compound of the present invention and a concomitant drug can be appropriately determined depending on the administration subject, administration route, target disease, symptom, combination and the like. When the subject of administration is human, for example, a concomitant drug can be used in 0.01-100 parts by weight relative to 1 part by weight of the compound of the present invention.

### Examples

The present invention is explained in detail in the following by referring to Reference Examples, Examples, Formulation Examples and Experimental Examples, which are mere embodiments and do not limit the present invention. The present invention can be modified without deviating from the scope of the invention.

In the following Reference Examples and Examples, the “room temperature” is generally about 10° C. to about 35° C. % means mol/mol % when it is used for the yield, % by volume for the solvent used for chromatography, and wt % for others. The compounds which are broad and cannot be confirmed by proton NMR spectrum, such as OH, NH proton etc., are not included in the data.

Other abbreviations used in the specification mean the following.

s: singlet

d: doublet

dd: doublet of doublets

dt: doublet of triplets

t: triplet

tt: triplet of triplets

td: triplet of doublets

q: quartet

septet: septet

m: multiplet

br: broad

J: coupling constant

Hz: Hertz

CDCl₃: deuterated chloroform

DMSO-d₆: deuterated dimethyl sulfoxide

¹H-NMR: proton nuclear magnetic resonance

HPLC: high performance liquid chromatography

THF: tetrahydrofuran

DMF: dimethylformamide

DMSO: dimethyl sulfoxide

HOBt: 1-hydroxybenzotriazole

WSC: 1-ethyl-3-(3-dimethylaminopropyl)carbodiimide hydrochloride

DBU: 1,8-diazabicyclo[5.4.0]-7-undecene

LC-MS: liquid chromatography-mass spectrometry spectrum

ESI: electrospray ionization method

### Reference Example 1

### 2-fluoro-4-iodopyridine

A 1.6 M n-butyllithium-hexane solution (19 ml, 30.5 mmol) was added dropwise to a solution (95 ml) of diisopropylamine (9.84 ml, 70 mmol) in tetrahydrofuran at −78° C., and the mixture was stirred for 30 min under an argon atmosphere. 2-Fluoropyridine (6.8 g, 30.5 mmol) was added dropwise to the mixture, and the mixture was stirred for 4 hr at −78° C. A solution (60 ml) of iodine (8.9 g, 30.5 mmol) in tetrahydrofuran was added, and the mixture was stirred for 1 hr. Water was added to the mixture, and the mixture was extracted with diethyl ether. The extract was washed with water, and dried over anhydrous sodium hydrogensulfate. The solvent was evaporated under reduced pressure. A 1.6 M n-butyllithium-hexane solution (19 ml, 30.5 mmol) was added dropwise to a solution (95 ml) of diisopropylamine (9.84 ml, 70 mmol) in tetrahydrofuran at −78° C., and the mixture was stirred for 30 min under an argon atmosphere. A solution (15 ml) of the obtained residue in tetrahydrofuran was added dropwise to the mixture, and the mixture was stirred at −78° C. for 1 hr under an argon atmosphere. Water was added to the mixture, and the mixture was extracted with diethyl ether. The extract was washed with water, and dried over anhydrous sodium hydrogensulfate. The solvent was evaporated under reduced pressure. The obtained residue was purified by silica gel column chromatography (hexane:ethyl acetate=9:1) to give the title compound (3.4 g, yield 28%) as a solid.

¹H-NMR (CDCl₃) δ: 7.37 (1H, d, J=1.7 Hz), 7.54 (1H, d, J=5.2 Hz), 7.92 (1H, dd, J=5.2, 1.7 Hz).

### Reference Example 2

### 4-iodopyridin-2-amine

2-Fluoro-4-iodopyridine (11.2 g, 50 mmol) obtained in Reference Example 1 and 28% aqueous ammonia solution (100 ml) were stirred at 150° C. for 3 hr in a sealed tube. The mixture was extracted with ethyl acetate. The extract was washed with water, and dried over anhydrous sodium hydrogensulfate. The solvent was evaporated under reduced pressure. The obtained residue was crystallized from ethyl acetate to give the title compound (6.6 g, yield 60%). melting point 167-168° C.

¹H-NMR (CDCl₃) δ: 4.34 (2H, brs), 6.92 (1H, d, J=1.4 Hz), 6.99 (1H, dd, J=5.5, 1.4 Hz), 7.73 (1H, d, J=5.5 Hz).

### Reference Example 3

### 1-(2-aminopyridin-4-yl)pyrrolidin-2-one

A solution of 4-iodopyridin-2-amine (440 mg, 2 mmol) obtained in Reference Example 2, pyrrolidin-2-one (387 mg, 2.4 mmol), potassium carbonate (553 mg, 4 mmol), copper iodide (76 mg, 0.4 mmol) and N,N′-dimethylethylenediamine (43 μl, 0.4 mmol) in dioxane (10 ml) was stirred at 100° C. for 14 hr under an argon atmosphere. Water was added to the mixture, and the mixture was extracted with ethyl acetate. The extract was washed with water, and dried over anhydrous sodium hydrogensulfate. The solvent was evaporated under reduced pressure. The residue was crystallized from ethanol to give the title compound (160 mg, yield 45%).

¹H-NMR (CDCl₃) δ: 2.17 (2H, tt, J=7.8, 6.9 Hz), 2.62 (2H, t, J=7.8 Hz), 3.82 (2H, t, J=6.9 Hz), 4.44 (2H, brs), 6.78 (1H, d, J=5.8, 1.9 Hz), 7.09 (1H, d, J=1.9 Hz), 8.01 (1H, d, J=5.8 Hz).

### Reference Example 4

### 1-(2-aminopyridin-4-yl)-4-phenylpyrrolidin-2-one

In the same manner as in Reference Example 3 and using 4-phenylpyrrolidin-2-one, the title compound was obtained. yield 55%.

¹H-NMR (DMSO-d₆) δ: 2.80 (1H, dd, J=17.1, 9.0 Hz), 3.01(1H, dd, J=17.1, 9.0 Hz), 3.68 (1H, m), 3.81 (1H, dd, J=9.4, 7.7 Hz), 4.16 (1H, dd, J=9.4, 8.3 Hz), 4.49 (1H, brs), 6.78 (1H, dd, J=5.8, 1.9 Hz), 7.08 (1H, d, J=1.9 Hz), 7.25-7.40 (5H, m), 8.00(1H, d, J=5.8 Hz).

### Reference Example 5

### 5-phenyl-1,3-oxazolidin-2-one

A mixture of 2-amino-1-phenylethanol (2.58 g, 18.8 mmol) and N,N′-carbonyldiimidazole (6.1 g, 37.6 mmol) in tetrahydrofuran (50 mL) was stirred at room temperature for 16 hr. The mixture was diluted with water, and extracted with ethyl acetate. The extract was washed with water, and dried over anhydrous sodium sulfate. The solvent was evaporated under reduced pressure, and the obtained residue was purified by silica gel column chromatography (hexane-ethyl acetate 1:4) to give the title compound (500 mg, yield 16%) as crystals. melting point 86-87° C. (ethyl acetate-hexane)

¹H-NMR (CDCl₃) δ: 3.54 (1H, t, J=8.7 Hz), 3.98 (1H, t, J=8.7 Hz), 5.61 (1H, t, J=8.1 Hz), 6.46 (1H,brs), 7.30-7.49 (5H, m).

### Reference Example 6

### N-benzyl-N′-(4-iodopyridin-2-yl)urea

In the same manner as in Example 1 and using 4-iodopyridin-2-amine, the title compound was obtained. yield 90%.

¹H-NMR (DMSO-d₆) δ: 4.37 (2H, d, J=6.0 Hz), 7.16-7.40 (6H, m), 7.90 (1H, d, J=5.2 Hz), 7.95 (1H, d, J=1.1 Hz), 8.20 (1H, brs), 9.33 (1H, brs).

### Reference Example 7

### ethyl 3-(4-methoxyphenyl)-4-nitrobutanoate

To a solution (2.23 ml) of ethyl 3-(4-methoxyphenyl)acrylate (2.06 g, 10 mmol) in nitromethane was added dropwise DBU (1.49 ml, 10 mmol) at 0° C., and the mixture was stirred at room temperature for 3 hr. The mixture was diluted with water, and extracted with diethyl ether. The extract was washed with water, and dried over anhydrous magnesium sulfate. The solvent was evaporated under reduced pressure, and the obtained residue was purified by silica gel column chromatography (hexane:ethyl acetate=3:1) to give the title compound (2.23 g, yield 82%) as an oil.

¹H-NMR (DMSO-d₆) δ: 1.17 (3H, t, J=7.1 Hz), 2.72 (2H, dd, J=7.4, 2.4 Hz), 3.77 (3H, s), 3.85-4.01 (1H, m), 4.08 (2H, q, J=7.1 Hz), 4.58 (1H, dd, J=12.4, 8.0 Hz), 4. 69 (1H, dd, J=12.4, 7.1 Hz), 6.85 (2H, d, J=8.5 Hz), 7.15 (2H, q, J=8.5 Hz).

### Reference Example 8

### 4-(4-methoxyphenyl)pyrrolidin-2-one

To a solution (70 ml) of ethyl 3-(4-methoxyphenyl)-4-nitrobutanoate (2 g, 7.48 mmol) obtained in Reference Example 7 in ethanol were added ammonium formate (3 g) and 10% palladium/carbon (0.5 g), and the mixture was stirred for 3 hr under heated reflux. The catalyst was filtered off, and the solvent was evaporated under reduced pressure. The obtained residue was dissolved in toluene (50 ml), the mixture was stirred at 120° C. for 3 hr, and the solvent was evaporated under reduced pressure. The obtained residue was purified by silica gel column chromatography (hexane:ethyl acetate=1:1) to give the title compound (800 mg, yield 45%) as a solid.

¹H-NMR (DMSO-d₆) δ: 2.47 (1H, dd, J=16.8, 8.8 Hz), 2.71 (1H, dd, J=16.8, 8.8 Hz), 3.38 (1H, dd, J=9.2, 7.3 Hz), 3.57-3.72 (1H, m), 3.76 (1H, td, J=8.8, 0.8 Hz), 3.80 (3H, s), 6.87 (2H, dd, J=8.5, 1.9 Hz), 7.17 (2H, dd, J=8.5, 1.9 Hz).

### Reference Example 9

### ethyl 3-(3-methylphenyl)acrylate

Ethyl diethylphosphonoacetate (11.2 ml, 55 mmol) was added to a solution (200 ml) of sodium hydride (2.2 g, 55 mmol) in DMF at 0° C., and the mixture was stirred for 30 min. 3-Methylbenzaldehyde (5.9 ml, 50 mmol) was added to the reaction mixture, and the mixture was stirred at room temperature for 1 hr. 1N Hydrochloric acid was added to the mixture, and the mixture was extracted with ethyl acetate. The extract was washed with water, and dried over anhydrous magnesium sulfate. The solvent was evaporated under reduced pressure, and the obtained residue was purified by silica gel column chromatography (hexane:ethyl acetate=3:1) to give the title compound (4.1 g, yield 42%).

¹H-NMR (CDCl₃) δ: 1.34 (3H, t, J=7.2 Hz), 2.37 (3H, s), 4.26 (2H, q, J=7.1 Hz), 6.42 (1H, d, J=15.9 Hz), 7.16-7.21 (1H, m), 7.26 (1H, t, J=7.7 Hz), 7.32 (2H, d, J=6.0 Hz), 7.66 (1H, dd, J=15.9 Hz).

### Reference Example 10

### ethyl 3-(3-methylphenyl)-4-nitrobutanoate

In the same manner as in Reference Example 7 and using ethyl 3-(3-methylphenyl)acrylate obtained in Reference Example 9, the title compound was obtained. yield 75%.

¹H-NMR (CDCl₃) δ: 1.18 (3H, t, J=7.0 Hz), 2.33 (3H, s), 2.74 (2H, d, J=7.4 Hz), 3.87-4.02 (1H, m), 4.08 (2H, q, J=7.1 Hz), 4.56-4.67 (1H, m), 4.67-4.77 (1H, m), 6.99-7.12 (3H, m), 7.21 (1H, t, J=7.4 Hz).

### Reference Example 11

### 4-(3-methylphenyl)pyrrolidin-2-one

In the same manner as in Reference Example 8 and using ethyl 3-(3-methylphenyl)-4-nitrobutanoate obtained in Reference Example 10, the title compound was obtained. yield 51%.

¹H-NMR (CDCl₃) δ: 2.27 (3H, s), 2.37-2.62 (2H, m), 3.06-3.24 (1H, m), 3.44-3.69 (2H, m), 6.92-7.13 (3H, m), 7.19 (1H, t, J=7.4 Hz), 7.68 (1H, s).

### Reference Example 12

### ethyl 3-(3-(trifluoromethyl)phenyl)acrylate

In the same manner as in Reference Example 9 and using 3-(trifluoromethyl)benzaldehyde, the title compound was obtained. yield 94%.

¹H-NMR (CDCl₃) δ: 1.33 (3H, t, J=7.1 Hz), 4.27 (2H, q, J=7.1 Hz), 6.49 (1H, d, J=15.9 Hz), 7.49 (1H, t, J=7.8 Hz), 7.56-7.73 (3H, m), 7.74 (1H, s).

### Reference Example 13

### ethyl 4-nitro-3-(3-(trifluoromethyl)phenyl)butanoate

In the same manner as in Reference Example 7 and using ethyl 3-(3-(trifluoromethyl)phenyl)acrylate obtained in Reference Example 12, the title compound was obtained. yield 74%.

¹H-NMR (CDCl₃) δ: 1.17 (3H, t, J=7.1 Hz), 2.77 (2H, dd, J=7.4, 3.6 Hz), 4.01-4.14 (3H, m), 4.58-4.72 (1H, m), 4.72-4.84 (1H, m), 7.39-7.53 (3H, m), 7.52-7.61 (1H, m).

### Reference Example 14

### 4-(3-(trifluoromethyl)phenyl)pyrrolidin-2-one

In the same manner as in Reference Example 8 and using ethyl 4-nitro-3-(3-(trifluoromethyl)phenyl)butanoate obtained in Reference Example 13, the title compound was obtained. yield 60%.

¹H-NMR (CDCl₃) δ: 2.49 (1H, dd, J=17.0, 8.3 Hz), 2.78 (1H, dd, J=17.0, 8.8 Hz), 3.43 (1H, dd, J=9.1, 6.6 Hz), 3.68-3.87 (2H, m), 7.05 (1H, brs), 7.43-7.56 (4H, m).

### Reference Example 15

### ethyl 3-(3-methoxyphenyl)acrylate

In the same manner as in Reference Example 9 and using 3-methoxybenzaldehyde, the title compound was obtained. yield 64%.

¹H-NMR (CDCl₃) δ: 1.34 (3H, t, J=7.1 Hz), 3.82 (3H, d, J=1.1 Hz), 4.26 (2H, q, J=7.2 Hz), 6.42 (1H, d, J=15.9 Hz), 6.92 (1H, dq, J=8.2, 1.1 Hz), 7.03 (1H, s), 7.10 (1H, d, J=7.7 Hz), 7.28 (1H, t, J=8.2 Hz), 7.64 (1H, d, J=15.9 Hz).

### Reference Example 16

### ethyl 3-(3-methoxyphenyl)-4-nitrobutanoate

In the same manner as in Reference Example 7 and using ethyl 3-(3-methoxyphenyl)acrylate obtained in Reference Example 15, the title compound was obtained. yield 43%.

¹H-NMR (CDCl₃) δ: 1.18 (3H t, J=7.1 Hz), 2.74 (2H, d, J=7.4 Hz), 3.78 (3H, s), 3.88-4.02 (1H, m), 4.08 (2H, q, J=7.1 Hz), 4.61 (1H, dd, J=12.6, 7.7 Hz), 4.71 (1H, dd, J=12.6, 7.2 Hz), 6.73-6.83 (3H, m), 7.23 (1H, t, J=8.0 Hz).

### Reference Example 17

### 4-(3-methoxyphenyl)pyrrolidin-2-one

In the same manner as in Reference Example 8 and using ethyl 3-(3-methoxyphenyl)-4-nitrobutanoate obtained in Reference Example 16, the title compound was obtained. yield 46%.

¹H-NMR (CDCl₃) δ: 2.50 (1H, dd, J=16.8, 8.8 Hz), 2.73 (1H, dd, J=16.5, 8.8 Hz), 3.42 (1H, dd, J=9.1, 7.2 Hz), 3.60-3.73 (1H, m), 3.81 (3H, s), 6.77-6.88 (3H, s), 7.21-7.30 (1H, m).

### Reference Example 18

### ethyl 3-(3-fluorophenyl)acrylate

In the same manner as in Reference Example 9 and using 3-fluorobenzaldehyde, the title compound was obtained. yield 78%.

¹H-NMR (CDCl₃) δ: 1.33 (3H, t, J=7.1 Hz), 4.26 (2H, q, J=7.1 Hz), 6.41 (1H, d, J=15.9 Hz), 7.06 (1H, tq, J=8.3, 1.4 Hz), 7.20 (1H, dt, J=7.8, 1.1 Hz), 7.33 (1H, dd, J=7.8, 5.6), 7.62 (1H, d, J=15.9 Hz).

### Reference Example 19

### ethyl 3-(3-fluorophenyl)-4-nitrobutanoate

In the same manner as in Reference Example 7 and using ethyl 3-(3-fluorophenyl)acrylate obtained in Reference Example 18, the title compound was obtained. yield 76%.

¹H-NMR (CDCl₃) δ: 1.19 (3H, t, J=7.1 Hz), 2.74 (2H, dd, J=1.2 Hz and J=7.6 Hz), 3.94-4.04 (1H, m), 4.09 (2H, q, J=7.1 Hz), 4.58-4.67 (1H, m), 4.69-4.77 (1H, m), 6.91-7.05 (3H, m), 7.26-7.35 (1H, m).

### Reference Example 20

### 4-(3-fluorophenyl)pyrrolidin-2-one

In the same manner as in Reference Example 8 and using ethyl 3-(3-fluorophenyl)-4-nitrobutanoate obtained in Reference Example 19, the title compound was obtained. yield 42%.

¹H-NMR (CDCl₃) δ: 2.37-2.54 (1H, m), 2.66-2.85 (1H, m), 3.27-3.51 (1H, m), 3.59-3.74 (1H, m), 3.75-3.86 (1H, m), 6.90-6.98 (2H, m), 7.02 (1H, dd, J=7.7 Hz), 7.21-7.40 (1H, m).

### Reference Example 21

### ethyl 3-(4-fluorophenyl)acrylate

In the same manner as in Reference Example 9 and using 4-fluorobenzaldehyde, the title compound was obtained. yield 100%.

¹H-NMR (CDCl₃) δ: 1.32 (3H, t, J=7.1 Hz), 4.25 (2H, q, J=7.1 Hz), 6.34 (1H, d, J=16.2 Hz), 7.05 (2H, t, J=8.9 Hz), 7.49 (2H, dd, J=8.9, 5.4 Hz), 7.63 (1H, d, J=16.2 Hz).

### Reference Example 22

### ethyl 3-(4-fluorophenyl)-4-nitrobutanoate

In the same manner as in Reference Example 7 and using ethyl 3-(4-fluorophenyl)acrylate obtained in Reference Example 21, the title compound was obtained. yield 62%.

¹H-NMR (CDCl₃) δ: 1.17 (3H, td, J=7.1, 1.9 Hz), 2.73 (2H, dd, J=7.4, 3.6 Hz), 4.02-4.16 (3H, m), 4.60 (1H, dd, J=12.4, 8.0 Hz), 4.71 (1H, dd, J=12.4, 6.6 Hz), 6.96-7.06 (2H, m), 7.16-7.25 (2H, m).

### Reference Example 23

### 4-(4-fluorophenyl)pyrrolidin-2-one

In the same manner as in Reference Example 8 and using ethyl 3-(4-fluorophenyl)-4-nitrobutanoate obtained in Reference Example 22, the title compound was obtained. yield 60%.

¹H-NMR (CDCl₃) δ: 2.19-2.34 (1H, m), 2.39-2.57 (1H, m), 3.04-3.22 (1H, m), 3.51-3.66 (2H, m), 7.01-7.19 (2H, m), 7.22-7.39 (2H, m), 7.68 (1H, s).

### Reference Example 24

### ethyl 3-(2-fluorophenyl)acrylate

In the same manner as in Reference Example 9 and using 2-fluorobenzaldehyde, the title compound was obtained. yield 100%.

¹H-NMR (CDCl₃) δ: 1.34 (3H, t, J=7.1 Hz), 4.26 (2H, q, J=7.2 Hz), 6.52 (1H, d, J=16.2 Hz), 7.03-7.18 (2H, m), 7.28-7.39 (1H, m), 7.52 (1H, td, J=7.6, 1.6 Hz), 7.80 (1H, d, J=16.2 Hz).

### Reference Example 25

### ethyl 3-(2-fluorophenyl)-4-nitrobutanoate

In the same manner as in Reference Example 7 and using ethyl 3-(2-fluorophenyl)acrylate obtained in Reference Example 24, the title compound was obtained. yield 100%.

¹H-NMR (CDCl₃) δ: 1.18 (3H, t, J=7.1 Hz), 2.83 (2H, dd, J=2.2 Hz and J=7.4 Hz), 4.03-4.19 (3H, m), 4.76 (2H, dd, J=3.4 Hz and J=7.3 Hz), 7.00-7.14 (2H, m), 7.14-7.34 (2H, m).

### Reference Example 26

### 4-(2-fluorophenyl)pyrrolidin-2-one

In the same manner as in Reference Example 8 and using ethyl 3-(2-fluorophenyl)-4-nitrobutanoate obtained in Reference Example 25, the title compound was obtained. yield 43%.

¹H-NMR (CDCl₃) δ: 2.38-2.65 (1H, m), 2.67-2.92 (1H, m), 3.35-0.56 (1H, m), 3.60-4.09 (2H, m), 7.02-7.17 (1H, m), 7.17-7.44 (3H, m).

### Reference Example 27

### N-(4-iodopyridin-2-yl)-N′-((pyridin-2-yl)methyl)urea

In the same manner as in Example 4 and using 4-iodopyridin-2-amine obtained in Reference Example 2, the title compound was obtained. yield 56%.

¹H-NMR (DMSO-d₆) δ: 4.45 (2H, d, J=5.5 Hz), 7.25 (1H, dd, J=7.7, 5.0 Hz), 7.28-7.34 (2H, m), 7.75 (1H, dd, J=7.7, 1.9 Hz), 7.90 (1H, d, J=5.2 Hz), 7.93 (1H, s), 8.36 (1H, brs), 8.50 (1H, d, J=5.0 Hz), 9.42 (1H, brs).

### Reference Example 28

### ethyl 1-(2-aminopyridin-4-yl)-3-benzyl-5-oxopyrrolidine-3-carboxylate

In the same manner as in Reference Example 3 and using ethyl 3-benzyl-5-oxopyrrolidine-3-carboxylate, the title compound was obtained. yield 53%.

¹H-NMR (DMSO-d₆) δ: 1.14 (3H, t, J=7.1 Hz), 2.75 (1H, d, J=17.0 Hz), 2.86 (1H, d, J=17.0 Hz), 3.08 (2H, s), 3.83 (1H, d, J=10.1 Hz), 4.00 (1H, d, J=10.2 Hz), 4.08 (2H, q, J=7.1 Hz), 5.87 (2H, s), 6.55-6.87 (2H, m), 7.06-7.17 (2H, m), 7.19-7.32 (3H, m), 7.81 (1H, s).

### Reference Example 29

### ethyl 5-phenylpent-2-enoate

In the same manner as in Reference Example 9 and using 3-phenylpropanal, the title compound was obtained. yield 100%.

¹H-NMR (CDCl₃) δ: 1.29 (3H, t, J=7.1 Hz), 2.47-2.59 (2H, m), 2.73-2.82 (2H, m), 4.19 (2H, q, J=7.2 Hz), 5.85 (1H, dt, J=15.7, 1.6 Hz), 7.01 (1H, dt, J=15.7, 6.9 Hz), 7.14-7.25 (3H, m), 7.24-7.34 (2H, m).

### Reference Example 30

### ethyl 3-(nitromethyl)-5-phenylpentanoate

In the same manner as in Reference Example 7 and using ethyl 5-phenylpent-2-enoate obtained in Reference Example 29, the title compound was obtained. yield 70%.

¹H-NMR (CDCl₃) δ: 1.28 (3H, t, J=7.1 Hz), 1.71-1.83 (2H, m), 2.47-2.53 (2H, m), 2.59-2.74 (3H, m), 4.16 (2H, q, J=7.1 Hz), 4.43-4.53 (1H, m), 4.52-4.61 (1H, m), 7.13-7.24 (3H, m), 7.26-7.34 (2H, m).

### Reference Example 31

### 4-(2-phenylethyl)pyrrolidin-2-one

In the same manner as in Reference Example 8 and using ethyl 3-(nitromethyl)-5-phenylpentanoate obtained in Reference Example 30, the title compound was obtained. yield 30%.

¹H-NMR (CDCl₃) δ: 1.80 (2H, q, J=7.5 Hz), 1.98-2.16 (1H, m), 2.28-2.71 (4H, m), 3.06 (1H, dd, J=9.6, 6.6 Hz), 3.44-3.55 (1H, m), 7.10-7.23 (3H, m), 7.29 (2H, t, J=7.2 Hz).

### Reference Example 32

### methyl 1-(4-methoxybenzyl)-5-oxopyrrolidine-3-carboxylate

A solution (15 ml) of dimethyl 2-methylenesuccinate (5.63 ml, 40 mmol) in methanol was added dropwise to a solution (50 ml) of 4-methoxybenzylamine (5.23 ml, 40 mmol) in methanol, and the mixture was stirred at room temperature for 3 days. The solvent was evaporated under reduced pressure, and the obtained residue was purified by silica gel column chromatography (hexane:ethyl acetate=5:1) to give the title compound (8.2 g, yield 78%) as an oil.

¹H-NMR (CDCl₃) δ: 2.67-2.78 (2H, m), 3.14-3.23 (1H, m), 3.45 (2H, d, J=7.7 Hz), 3.70 (3H, s), 3.80 (3H, s), 4.31-4.48 (2H, m), 6.86 (2H, d, J=8.5 Hz), 7.17 (2H, d, J=8.5 Hz).

### Reference Example 33

### 4-(hydroxymethyl)-1-(4-methoxybenzyl)pyrrolidin-2-one

To a solution (100 ml) of methyl 1-(4-methoxybenzyl)-5-oxopyrrolidine-3-carboxylate (5.27 g, 20 mmol) obtained in Reference Example 32 in ethanol was added sodium tetrahydroborate (7.57 g, 200 mmol) at 0° C., and the mixture was stirred at room temperature for 12 hr. The mixture was diluted with water, and the insoluble material was filtered off. The filtrate was evaporated under reduced pressure, and tetrahydrofuran and water were added to the obtained residue. The mixture was stirred for 4 hr, and extracted with ethyl acetate. The extract was washed with water, and dried over anhydrous magnesium sulfate. The solvent was evaporated to give the title compound (4.0 g, yield 85%) as a solid.

¹H-NMR (CDCl₃) δ: 2.16-2.36 (1H, m), 2.44-2.61 (3H, m), 3.08 (1H, dd, J=9.9, 4.9 Hz), 3.33 (1H, dd, J=9.9, 8.0 Hz), 3.42-3.63 (2H, m), 3.73-3.81 (3H, m), 4.35 (2H, d, J=4.7 Hz), 6.78-6.92 (2H, m), 7.06-7.20 (2H, m).

### Reference Example 34

### 1-(4-methoxybenzyl)-4-(phenoxymethyl)pyrrolidin-2-one

To a solution (40 ml) of 4-(hydroxymethyl)-1-(4-methoxybenzyl)pyrrolidin-2-one (941 mg, 4 mmol) obtained in Reference Example 33 and triethylamine (0.35 ml, 4.8 mmol) in tetrahydrofuran was added methanesulfonyl chloride (0.37 ml, 4.8 mmol) at 0° C., and the mixture was stirred at room temperature for 3 hr. Water was added to the mixture, and the mixture was extracted with ethyl acetate. The extract was washed with water, and dried over anhydrous magnesium sulfate. The solvent was evaporated under reduced pressure. The obtained residue was added to a mixture (stirred for 30 min) of phenol (452 mg, 4.8 mmol) and a solution (40 ml) of sodium hydride (211 mg, 5.3 mmol) in tetrahydrofuran. The mixture was stirred for 6 hr while heating under reflux. 1N Hydrochloric acid was added to the mixture, and the mixture was extracted with ethyl acetate. The extract was washed with water, and dried over anhydrous magnesium sulfate. The solvent was evaporated under reduced pressure, and the obtained residue was purified by silica gel column chromatography (hexane:ethyl acetate=3:1) to give the title compound (971 mg, yield 78%) as a solid.

¹H-NMR (CDCl₃) δ: 2.31-2.45 (1H, m), 2.58-2.73 (1H, m), 2.74-2.92 (1H, m), 3.19 (1H, dd, J=10.0, 5.1 Hz), 3.45 (1H, dd, J=10.0, 8.1 Hz), 3.75-3.81 (3H, m), 3.82-3.95 (2H, m), 4.32-4.49 (2H, m), 6.76-7.02 (5H, m), 7.10-7.34 (4H, m).

### Reference Example 35

### 4-(phenoxymethyl)pyrrolidin-2-one

A mixture of 1-(4-methoxybenzyl)-4-(phenoxymethyl)pyrrolidin-2-one (311 mg, 1 mmol) obtained in Reference Example 34, trifluoroacetic acid (4 ml) and methoxybenzene (0.8 ml) was stirred with irradiating microwave at 100° C. for 20 min. Saturated aqueous sodium hydrogen carbonate solution was added to the mixture, and the mixture was extracted with ethyl acetate. The extract was washed with water, and dried over anhydrous magnesium sulfate. The solvent was evaporated under reduced pressure, and the obtained residue was purified by silica gel column chromatography (hexane:ethyl acetate=1:1) to give the title compound (163 mg, yield 85%) as an oil.

¹H-NMR (CDCl₃) δ: 2.27 (1H, dd, J=17.2, 6.5 Hz), 2.55 (1H, dd, J=17.2, 6.5 Hz), 2.83-3.05 (1H, m), 3.36 (1H, dd, J=9.9, 5.5 Hz), 3.54-3.67 (1H, m), 3.85-4.07 (2H, m), 6.86-6.92 (2H, m), 6.97 (1H, td, J=7.3, 1.1 Hz), 7.24-7.34 (2H, m).

### Reference Example 36

### ethyl 3-(4-methoxyphenyl)propanoate

A mixture of ethyl 3-(4-methoxyphenyl)acrylate (2.01 g, 9.75 mmol), 10% palladium carbon (0.2 g) and ethanol (100 mL) was stirred at room temperature for 6 hr under a hydrogen atmosphere. The catalyst was filtered off, and the solvent was evaporated under reduced pressure to give the title compound (2.05 g, yield 100%) as an oil.

¹H-NMR (CDCl₃) δ: 1.24 (3H, t, J=7.1 Hz), 2.59 (2H, t, J=7.7 Hz), 2.89 (2H, t, J=7.7 Hz), 3.78 (3H, s), 4.12 (2H, q, J=7.1 Hz), 6.82 (2H, d, J=8.8 Hz), 7.12 (2H, d, J=8.8 Hz).

### Reference Example 37

### 3-(4-methoxyphenyl)propanal

Diisobutylaluminum hydride (7.03 ml, 10.54 mmol, 1.5 M toluene solution) was added to a solution (50 ml) of ethyl 3-(4-methoxyphenyl)propanoate (2.05 g, 9.85 mmol) obtained in Reference Example 36 in toluene at −78° C., and the mixture was stirred for 1 hr. Saturated aqueous ammonium chloride solution was added to the mixture, and the mixture was extracted with ethyl acetate. The extract was washed with water, and dried over anhydrous magnesium sulfate. The solvent was evaporated under reduced pressure, and the obtained residue was purified by silica gel column chromatography (hexane:ethyl acetate=5:1) to give the title compound (1.27 g, yield 79%) as an oil.

¹H-NMR (CDCl₃) δ: 2.74-2.79 (2H, m), 2.91 (2H, t, J=7.6 Hz), 3.79 (3H, s), 6.83 (9H, s), 7.11 (2H, d, J=8.8 Hz), 9.80 (1H, t, J=1.5 Hz).

### Reference Example 38

### ethyl 5-(4-methoxyphenyl)pent-2-enoate

In the same manner as in Reference Example 9 and using 3-(4-methoxyphenyl)propanal obtained in Reference Example 37, the title compound was obtained. yield 100%.

¹H-NMR (CDCl₃) δ: 1.29 (3H, t, J=7.1 Hz), 2.44-2.54 (2H, m), 2.72 (2H, t, J=7.7 Hz), 3.79 (3H, s), 4.18 (2H, q, J=7.1 Hz), 5.83 (1H, dt, J=15.7, 1.5 Hz), 6.83 (2H, d, J=8.8 Hz), 6.99 (1H, dt, J=15.7, 6.8 Hz), 7.09 (2H, d, J=8.8 Hz).

### Reference Example 39

### ethyl 5-(4-methoxyphenyl)-3-(nitromethyl)pentanoate

In the same manner as in Reference Example 7 and using ethyl 5-(4-methoxyphenyl)pent-2-enoate obtained in Reference Example 38, the title compound was obtained. yield 79%.

¹H-NMR (CDCl₃) δ: 1.27 (3H, t, J=7.1 Hz), 1.66-1.79 (2H, m), 2.45-2.51 (2H, m), 2.59-2.68 (3H, m), 3.79 (3H, s), 4.16 (2H, q, J=7.1 Hz), 4.47 (1H, dd, J=12.1, 6.1 Hz), 4.55 (1H, dd, J=12.1, 6.6 Hz), 6.83 (2H, d, J=8.8 Hz), 7.07 (2H, d, J=8.8 Hz).

### Reference Example 40

### 4-(2-(4-methoxyphenyl)ethyl)pyrrolidin-2-one

In the same manner as in Reference Example 8 and using ethyl 5-(4-methoxyphenyl)-3-(nitromethyl)pentanoate obtained in Reference Example 39, the title compound was obtained. yield 49%.

¹H-NMR (CDCl₃) δ: 1.73-1.83 (1H, m), 1.96-2.12 (1H, m), 2.38-2.50 (2H, m), 2.58 (2H, td, J=7.8, 3.2 Hz), 3.04 (1H, dd, J=9.5, 6.7 Hz), 3.42-3.52 (1H, m), 3.79 (3H, s), 6.83 (2H, d, J=8.8 Hz), 7.07 (2H, d, J=8.8 Hz).

### Reference Example 41

### 5-phenyl-1,3-oxazolidin-2-one

A mixture of 2-amino-1-phenylethanol (2.58 g, 18.8 mmol) and N,N′-carbonyldiimidazole (6.1 g, 37.6 mmol) in tetrahydrofuran (50 mL) was stirred at room temperature for 16 hr. The mixture was diluted with water, and extracted with ethyl acetate. The extract was washed with water, and dried over anhydrous sodium sulfate. The solvent was evaporated under reduced pressure, and the obtained residue was purified by silica gel column chromatography (hexane-ethyl acetate 1:4) to give the title compound (500 mg, yield 16%) as crystals.

melting point 86-87° C. (ethyl acetate-hexane)

¹H-NMR (CDCl₃) δ: 3.54 (1H, t, J=8.7 Hz), 3.98 (1H, t, J=8.7 Hz), 5.61 (1H, t, J=8.1 Hz), 6.46 (1H,br s), 7.30-7.49 (5H, m).

### Reference Example 42

### 3-(2-aminopyridin-4-yl)-5-phenyl-1,3-oxazolidin-2-one

To a solution of 4-iodopyridin-2-amine (2.0 g, 9.09 mmol), 5-phenyl-1,3-oxazolidin-2-one (1.7 g, 10.5 mmol) obtained in Reference Example 41 and potassium carbonate (2.5 g, 18.2 mmol) in 1,4-dioxane (30 mL) were added copper iodide (173 mg, 909 μmol) and N,N′-dimethylethylenediamine (80 mg, 909 μmol) at room temperature, and the mixture was heated under reflux for 16 hr under an argon stream. The solid was filtered off, and the filtrate was concentrated under reduced pressure. The obtained residue was purified by silica gel column chromatography (ethyl acetate) to give the title compound (1.78 g, yield 77%) as crystals.

melting point 155-156° C. (ethyl acetate-hexane)

¹H-NMR (CDCl₃) δ: 3.90 (1H, dd, J=9.0, 7.5 Hz), 4.35 (1H, t, J=9.0 Hz), 4.49 (2H, br s), 5.64 (1H, t, J=7.5 Hz), 6.67 (1H, dd, J=6.0, 2.1 Hz), 6.93 (1H, d, J=2.1 Hz), 7.34-7.50 (5H, m), 8.00 (1H, d, J=6.0 Hz).

### Reference Example 43

### 5-benzyl-1,3-oxazolidin-2-one

To a mixture of 2-benzyloxirane (2.5 g, 18.6 mmol) and methyl carbamate (1.68 g, 22.3 mmol) was added triethylamine (0.5 mL), and the mixture was stirred at 120° C. for 3 hr. The mixture was allowed to cool to room temperature, and diluted with ethyl acetate. This solution was washed with water, dried over anhydrous magnesium sulfate, and concentrated under reduced pressure. The obtained residue was purified by basic silica gel column chromatography (ethyl acetate) to give the title compound (1.1 g, yield 33%) as crystals. melting point 104-105° C. (ethyl acetate-hexane)

¹H-NMR (CDCl₃) δ: 2.95 (1H, dd, J=14.1, 6.9 Hz), 3.16 (1H, dd, J=14.1, 6.0 Hz), 3.33 (1H, t, J=7.8 Hz), 3.58 (1H, t, J=8.4 Hz), 4.81-4.92 (1H, m), 5.58 (1H, br s), 7.20-7.43 (5H, m).

### Reference Example 44

### 3-(2-aminopyridin-4-yl)-5-benzyl-1,3-oxazolidin-2-one

In the same manner as in Reference Example 42 and using 5-benzyl-1,3-oxazolidin-2-one obtained in Reference Example 43, the title compound was obtained as crystals. yield 70%.

melting point 181-182° C. (ethyl acetate-hexane)

¹H-NMR (CDCl₃) δ: 3.00 (1H, dd, J=13.8, 6.9 Hz), 3.23 (1H, dd, J=13.8, 6.0 Hz), 3.67 (1H, dd, J=9.0, 6.9 Hz), 3.93 (1H, t, J=8.4 Hz), 4.43 (2H, br s), 4.83-4.95 (1H, m), 6.61 (1H, dd, J=6.0, 2.1 Hz), 6.84 (1H, d, J=2.1 Hz), 7.22-7.39 (5H, m), 7.97 (1H, d, J=6.0 Hz).

### Reference Example 45

### 5-(2-phenylethyl)-1,3-oxazolidin-2-one

In the same manner as in Reference Example 43 and using 2-(2-phenylethyl)oxirane, the title compound was obtained as crystals. yield 53%.

melting point 82-83° C. (ethyl acetate-hexane)

¹H-NMR (CDCl₃) δ: 1.85-2.01 (1H, m), 2.05-2.23 (1H, m), 2.67-2.90 (2H, m), 3.23 (1H, t, J=8.1 Hz), 3.64 (1H, t, J=8.7 Hz), 4.56-4.67 (1H, m), 5.48 (1H, brs), 7.17-7.34 (5H, m).

### Reference Example 46

### 3-(2-aminopyridin-4-yl)-5-(2-phenylethyl)-1,3-oxazolidin-2-one

In the same manner as in Reference Example 42 and using 5-(2-phenylethyl)-1,3-oxazolidin-2-one obtained in Reference Example 45, the title compound was obtained as crystals. yield 80%.

melting point 143-144° C.

¹H-NMR (CDCl₃) δ: 1.93-2.28 (2H, m), 2.70-2.97 (2H, m), 3.56 (1H, dd, J=8.7, 7.2 Hz), 3.98 (1H, t, J=8.7 Hz), 4.47 (2H, br s), 4.58-4.65 (1H, m), 6.62 (1H, dd, J=6.0, 2.1 Hz), 6.89 (1H, d, J=2.1 Hz), 7.12-7.34 (5H, m), 7.98 (1H, d, J=6.0 Hz).

### Reference Example 47

### 5-(phenoxymethyl)-1,3-oxazolidin-2-one

In the same manner as in Reference Example 43 and using 2-(phenoxymethyl)oxirane, the title compound was obtained as crystals. yield 47%.

melting point 108-109° C. (ethyl acetate-hexane)

¹H-NMR (CDCl₃) δ: 3.64 (1H, dd, J=8.4, 6.0 Hz), 3.79 (1H, t, J=8.4 Hz), 4.09-4.22 (2H, m), 4.95-5.05 (1H, m), 5.40 (1H, brs), 6.91 (2H, d, J=7.8 Hz), 6.92-7.03 (1H, m), 7.24-7.35 (2H, m).

### Reference Example 48

### 3-(2-aminopyridin-4-yl)-5-(phenoxymethyl)-1,3-oxazolidin-2-one

In the same manner as in Reference Example 42 and using 5-(phenoxymethyl)-1,3-oxazolidin-2-one obtained in Reference Example 47, the title compound was obtained as crystals. yield 74%.

melting point 191-192° C.

¹H-NMR (CDCl₃) δ: 4.00-4.23 (4H, m), 4.47 (2H, br s), 4.90-5.10 (1H, m), 6.71 (1H, d, J=5.7 Hz), 6.86-7.03 (4H, m), 7.24-7.33 (2H, m), 8.02 (1H, d, J=5.7 Hz).

### Reference Example 49

### 5-((benzyloxy)methyl)-1,3-oxazolidin-2-one

In the same manner as in Reference Example 43 and using 2-((benzyloxy)methyl)oxirane, the title compound was obtained as an oil. yield 52%.

¹H-NMR (CDCl₃) δ: 3.45-3.70 (4H, m), 4.59 (2H, s), 4.72-4.83 (1H, m), 5.28 (1H, brs), 7.20-7.42 (5H, m).

### Reference Example 50

### 3-(2-aminopyridin-4-yl)-5-((benzyloxy)methyl)-1,3-oxazolidin-2-one

In the same manner as in Reference Example 42 and using 5-((benzyloxy)methyl)-1,3-oxazolidin-2-one obtained in Reference Example 49, the title compound was obtained as crystals. yield 79%. melting point 122-123° C.

¹H-NMR (CDCl₃) δ: 3.65-3.76 (2H, m), 3.87 (1H, dd, J=9.0, 6.0 Hz), 4.00 (1H, t, J=9.0 Hz), 4.49 (2H, br s), 4.58 (1H, d, J=12.0 Hz), 4.63 (1H, d, J=12.0 Hz), 4.64-4.83 (1H, m), 6.68 (1H, dd, J=5.7, 2.1 Hz), 6.91 (1H, s), 7.26-7.40 (5H, m), 8.00 (1H, d, J=5.4 Hz).

### Reference Example 51

### 3-(2-aminopyridin-4-yl)-5-phenyl-1,3-oxazol-2(3H)-one

A solution of 4-iodopyridin-2-amine (500 mg, 2.27 mmol), 5-phenyl-2-oxazoline (438 mg, 2.72 mmol), copper iodide (85.7 mg, 0.45 mmol), N,N′-dimethylcyclohexane-1,2-diamine (71.0 μl, 0.45 mmol) and potassium carbonate (627 mg, 4.54 mmol) in 1,4-dioxane (10 ml) was stirred with irradiating microwave (250 W) at 100° C. for 30 min. The reaction mixture was filtered, and the filtrate was evaporated under reduced pressure. The residue was purified by silica gel column chromatography (ethyl acetate) to give the title compound (120 mg, yield 21%) as a solid.

¹H-NMR (DMSO-d₆): δ 6.20 (2H, s), 6.82 (1H, dd, J=5.7, 2.1 Hz), 7.04 (1H, d, J=1.7 Hz), 7.33-7.41 (1H, m), 7.44-7.52 (2H, m), 7.60-7.67 (2H, m), 7.99 (1H, d, J=5.7 Hz), 8.30 (1H, s).

### Reference Example 52

### 3-(2-aminopyridin-4-yl)-5-(2-thienyl)-1,3-oxazolidin-2-one

In the same manner as in Reference Example 42 and using 5-(2-thienyl)-1,3-oxazolidin-2-one (Journal of Medicinal Chemistry, vol. 11, pages 1121-1125, 1968), the title compound was obtained as crystals. yield 56%.

melting point 151-153° C. (ethyl acetate-hexane)

¹H-NMR (CDCl₃) δ: 4.06 (1H, dd, J=9.0, 7.5 Hz), 4.34 (1H, t, J=9.0 Hz), 4.53 (2H, br s), 5.87 (1H, t, J=7.8 Hz), 6.68 (1H, dd, J=5.7, 1.8 Hz), 6.92 (1H, d, J=1.5 Hz), 7.05 (1H, dd, J=5.1, 3.3 Hz), 7.18-7.21 (1H, m), 7.41 (1H, dd, J=5.1, 1.5 Hz), 8.03 (1H, d, J=5.1 Hz).

### Reference Example 53

### ethyl 3,3-dimethyl-4-nitrobutanoate

In the same manner as in Reference Example 7 and using ethyl 3-methylbut-2-enoate, the title compound was obtained. yield 98%.

¹H-NMR (CDCl₃) δ: 1.16 (6H, s), 1.26 (3H, t, J=7.1 Hz), 2.43 (2H, s), 4.13 (2H, q, J=7.1 Hz), 4.52 (2H, s).

### Reference Example 54

### 4,4-dimethylpyrrolidin-2-one

In the same manner as in Reference Example 8 and using ethyl 3,3-dimethyl-4-nitrobutanoate obtained in Reference Example 53, the title compound was obtained. yield 25%.

¹H-NMR (CDCl₃) δ: 1.15 (6H s), 2.40 (2H, s), 3.55 (2H, s).

### Reference Example 55

### 2-phenyl-2-((trimethylsilyl)oxy)propanenitrile

To a mixture of acetophenone (9.7 g, 80.7 mmol) and zinc iodide (2.34 g, 7.33 mmol) was added trimethylsilyl cyanide (25 mL, 187 mmol), and the mixture was stirred for 3 hr. The mixture was diluted with water, and extracted with ethyl acetate. The extract was washed with water, and dried over anhydrous sodium sulfate. The solvent was evaporated under reduced pressure to give the title compound (14.3 g, yield 81%) as an oil.

¹H-NMR (CDCl₃) δ: 0.19 (9H, s), 1.87 (3H, s), 7.30-7.47 (3H, m), 7.52-7.58 (2H, m).

### Reference Example 56

### 5-methyl-5-phenyl-1,3-oxazolidin-2-one

To a solution (100 ml) of 2-phenyl-2-((trimethylsilyl)oxy)propanenitrile (14.3 g, 65.2 mmol) obtained in Reference Example 55 in tetrahydrofuran was added lithium aluminum hydride (2.97 g, 78.2 mmol) in small portions under ice-cooling, and the mixture was stirred at room temperature for 2 hr and at 50° C. for 1 hr. The reaction mixture was poured into ice water, and the resulting product was extracted with ethyl acetate. The extract was washed with water, dried over anhydrous magnesium sulfate, filtered, and concentrated under reduced pressure. The residue was crystallized from methanol to give 1-amino-2-phenylpropan-2-ol (11 g). A mixture of this compound and N,N′-carbonyldiimidazole (13.0 g, 80.0 mmol) in tetrahydrofuran (100 mL) was stirred at room temperature for 60 hr. The mixture was diluted with water, and extracted with ethyl acetate. The extract was washed with water, and dried over anhydrous sodium sulfate. The solvent was evaporated under reduced pressure, and the obtained residue was crystallized from hexane-ethyl acetate to give the title compound (3.8 g, yield 33%) as crystals.

melting point 144-145° C. (ethyl acetate-hexane)

¹H-NMR (CDCl₃) δ: 1.78 (3H, s), 3.69 (1H, d, J=9.3 Hz), 3.73 (1H, d, J=9.0 Hz), 6.14 (1H, brs), 7.25-7.50 (5H, m).

### Reference Example 57

### 3-(2-aminopyridin-4-yl)-5-methyl-5-phenyl-1,3-oxazolidin-2-one

In the same manner as in Reference Example 42 and using 5-methyl-5-phenyl-1,3-oxazolidin-2-one obtained in Reference Example 56, the title compound was obtained as crystals. yield 79%.

melting point 140-141° C.

H-NMR (CDCl₃) δ: 1.86 (3H, s), 4.05 (1H, d, J=8.4 Hz), 4.09 (1H, d, J=9.0 Hz), 4.48 (2H, br s), 6.64 (1H, dd, J=6.0, 1.8 Hz), 6.90 (1H, d, J=1.8 Hz), 7.28-7.50 (5H, m), 7.98 (1H, d, J=6.0 Hz).

### Reference Example 58

### ethyl (3-nitropyridin-2-yl)acetate

To a suspension of sodium hydride (60% dispersion in liquid paraffin, 4.24 g, 106 mmol) in DMSO (60 mL) was added diethyl malonate (17 g, 106 mmol) at 0° C. over 20 min in small portions, and the mixture was stirred at the same temperature for 1 hr. 2-Chloro-3-nitropyridine (7.2 g, 45.4 mmol) was added to the mixture, and the mixture was stirred at 100° C. for 15 min. Aqueous ammonium chloride solution was added to the reaction mixture, and the resulting product was extracted with ethyl acetate. The extract was washed with water, dried over anhydrous magnesium sulfate, and concentrated under reduced pressure to give a crude product (13 g) of diethyl (3-nitropyridin-2-yl)malonate. A mixture of this compound, lithium chloride (4.88 g, 115 mmol) and water (1 mL) in DMSO (150 mL) was stirred at 100° C. for 16 hr. The reaction mixture was diluted with saturated brine, and the resulting product was extracted with ethyl acetate. The extract was washed with water, dried over magnesium sulfate, and concentrated under reduced pressure. The obtained residue was purified by basic silica gel column chromatography (hexane:ethyl acetate=1:1) to give the title compound (6.4 g, yield 70%) as an oil.

¹H-NMR (CDCl₃) δ: 1.26 (3H, t, J=7.2 Hz), 4.19 (2H, q, J=7.2 Hz), 4.33 (2H, s), 7.48 (1H, dd, J=8.7, 5.1 Hz), 8.42 (1H, dd, J=8.7, 1.8 Hz), 8.79 (1H, dd, J=5.1, 1.8 Hz).

### Reference Example 59

### ethyl 2-methyl-2-(3-nitropyridin-2-yl)propanoate

To a solution of ethyl (3-nitropyridin-2-yl)acetate (6.4 g, 30.4 mmol) synthesized in Reference Example 58, methyl iodide (12.8 g, 91.3 mmol) and 18-crown-6 (804 mg, 3.04 mmol) in DMF (60 mL) was added sodium hydride (60% dispersion in liquid paraffin, 2.68 g, 5.45 mmol) at 5° C. over 20 min in small portions, and the mixture was stirred at the same temperature for 1 hr. Water was added to the reaction mixture, and the resulting product was extracted with ethyl acetate. The combined extracts were washed with water, dried over anhydrous magnesium sulfate, and concentrated under reduced pressure. The obtained residue was purified by silica gel column chromatography (hexane:ethyl acetate=3:2) to give the title compound (6.63 g, yield 92%) as an oil.

¹H-NMR (CDCl₃) δ: 1.20 (3H, t, J=7.2 Hz), 1.72 (6H, s), 4.12 (2H, q, J=7.2 Hz), 7.40 (1H, dd, J=8.1, 4.8 Hz), 8.23 (1H, dd, J=8.1, 1.5 Hz), 8.78 (1H, dd, J=1.8, 1.5 Hz).

### Reference Example 60

### 3,3-dimethyl-1,3-dihydro-2H-pyrrolo[3,2-b]pyridin-2-one

A mixture of ethyl 2-methyl-2-(3-nitropyridin-2-yl)propanoate (6.63 g, 27.8 mmol) obtained in Reference Example 59, 10%-palladium carbon (50% containing water, 600 mg) and ammonium formate (7.1 g, 111 mmol) in ethanol (50 mL) was heated under reflux for 2 hr. The solid was filtered off, and the filtrate was concentrated under reduced pressure. Water and ethyl acetate were added to the residue, the organic layer was separated, and the aqueous layer was extracted with ethyl acetate. The combined organic layers were washed with water, and dried over anhydrous sodium sulfate. The solvent was evaporated under reduced pressure, and the obtained residue was crystallized from ethyl acetate-hexane to give the title compound (3.5 g, yield 78%) as crystals.

melting point 148-149° C. (ethyl acetate-hexane)

¹H-NMR (DMSO-d₆) δ: 1.25 (3H, s), 1.31 (3H, s), 7.10-7.37 (2H, m), 8.06-8.17 (1H, m), 10.5 (0.5H, br s), 10.9 (0.5H, br s).

### Reference Example 61

### 1-(2-aminopyridin-4-yl)-3,3-dimethyl-1,3-dihydro-2H-pyrrolo[3,2-b]pyridin-2-one

In the same manner as in Reference Example 42 and using 3,3-dimethyl-1,3-dihydro-2H-pyrrolo[3,2-b]pyridin-2-one obtained in Reference Example 60, the title compound was obtained as crystals. yield 78%.

melting point 210-211° C. (ethyl acetate-hexane)

¹H-NMR (CDCl₃) δ: 1.53 (6H, s), 4.62 (2H, br s), 6.67 (1H, d, J=1.8 Hz), 6.75 (1H, dd, J=5.7, 1.8 Hz), 7.16 (1H, dd, J=8.1, 5.1 Hz), 7.33 (1H, dt, J=8.1, 0.6 Hz), 8.22 (1H, d, J=5.7 Hz), 8.31 (1H, d, J=5.1 Hz).

### Reference Example 62

### 1′-(2-aminopyridin-4-yl)spiro(cyclopentane-1,3′-indol)-2′(1′H)-one

In the same manner as in Reference Example 3, and using 4-iodopyridin-2-amine and spiro(cyclopentane-1,3′-indol)-2′(1′H)-one, the title compound was obtained. yield 45%.

¹H-NMR (DMSO-d₆) δ: 1.76-2.16 (8H, m), 6.17 (2H, s), 6.94 (1H, d, J=7.7 Hz), 7.11 (1H, t, J=7.3 Hz), 7.23 (1H, td, J=7.8, 1.2 Hz), 7.38 (1H, dd, J=7.3, 1.0 Hz), 8.04 (1H, brs).

### Reference Example 63

### 1-methyl-1,3-dihydro-2H-benzimidazol-2-one

A mixture of N-methylbenzene-1,2-diamine (4.0 g, 8.76 mmol), N,N′-carbonyldiimidazole (2.84 g, 17.5 mmol) and pyridine (1.41 mL, 17.5 mmol) in tetrahydrofuran (30 mL) was stirred at 65° C. for 1 hr. The mixture was diluted with water, and extracted with ethyl acetate. The extract was washed with water, and dried over anhydrous sodium sulfate. The solvent was evaporated under reduced pressure, and the obtained residue was crystallized from hexane-ethyl acetate to give the title compound (3.0 g, yield 67%) as crystals.

melting point 191-192° C. (ethyl acetate-hexane)

¹H-NMR (CDCl₃) δ: 3.44 (3H, s), 6.94-7.15 (4H, m), 10.4 (1H, br s).

### Reference Example 64

### 1-(2-aminopyridin-4-yl)-3-methyl-1,3-dihydro-2H-benzimidazol-2-one

In the same manner as in Reference Example 42 and using 1-methyl-1,3-dihydro-2H-benzimidazol-2-one obtained in Reference Example 63, the title compound was obtained as crystals. yield 80%

melting point 204-205° C. (ethyl acetate-hexane)

¹H-NMR (CDCl₃) δ: 3.48 (3H, s), 4.61 (2H, br s), 6.81 (1H, d, J=1.8 Hz), 6.11 (1H, dd, J=5.4, 1.8 Hz), 7.01-7.31 (4H, m), 8.19 (1H, d, J=5.4 Hz).

### Reference Example 65

### 1-phenyl-1,3-dihydro-2H-benzimidazol-2-one

In the same manner as in Reference Example 64 and using N-phenylbenzene-1,2-diamine, the title compound was obtained as crystals. yield 76%.

melting point 202-203° C. (ethyl acetate-hexane)

¹H-NMR (CDCl₃) δ: 7.02-7.17 (4H, m), 7.40-7.46 (1H, m), 7.53-7.58 (4H, m), 10.1 (1H, br s).

### Reference Example 66

### 1-(2-aminopyridin-4-yl)-3-phenyl-1,3-dihydro-2H-benzimidazol-2-one

In the same manner as in Reference Example 42 and using 1-phenyl-1,3-dihydro-2H-benzimidazol-2-one obtained in Reference Example 65, the title compound was obtained as crystals. yield 78%. melting point 184-185° C. (ethyl acetate-hexane)

¹H-NMR (CDCl₃) δ: 4.67 (2H, br s), 6.86 (1H, s), 6.96 (1H, dd, J=5.7, 1.8 Hz), 7.02-7.20 (3H, m), 7.26-7.29 (1H, m), 7.40-7.65 (5H, m), 8.21 (1H, d, J=5.4 Hz).

### Reference Example 67

### N-tert-butylbenzene-1,2-diamine

To a solution of 1-fluoro-2-nitrobenzene (5.00 g, 35.4 mmol) in DMF (50 mL) were added tert-butylamine (12.9 g, 177 mmol) and potassium carbonate (9.68 g, 70 mmol) at room temperature, and the mixture was stirred at 60° C. for 3 days. The reaction mixture was allowed to cool to room temperature, poured into water, and extracted with ethyl acetate. The organic layer was washed with saturated brine, dried over anhydrous sodium sulfate, and concentrated under reduced pressure to give crude product of N-tert-butyl-2-nitroaniline (6.0 g). A mixture of this compound and 10%-palladium carbon (50% in water, 600 mg) and ammonium formate (4.41 g, 70 mmol) in ethanol (100 mL) was heated under reflux for 2 hr. The solid was filtered off, and the filtrate was concentrated under reduced pressure. Water and ethyl acetate were added to the residue, the organic layer was separated, and the aqueous layer was extracted with ethyl acetate. The combined organic layers were washed with water, and dried over anhydrous sodium sulfate. The solvent was evaporated under reduced pressure to give the title compound (5.1 g, yield 88%).

¹H-NMR (CDCl₃) δ: 1.31 (9H, m), 2.80 (1H, brs), 3.60 (2H, brs), 6.60-7.02 (4H, m).

### Reference Example 68

### 1-tert-butyl-1,3-dihydro-2H-benzimidazol-2-one

In the same manner as in Reference Example 63 and using N-tert-butylbenzene-1,2-diamine obtained in Reference Example 67, the title compound was obtained as crystals. yield 56%.

melting point 146-147° C. (ethyl acetate-hexane)

¹H-NMR (CDCl₃) δ: 1.81 (9H, m), 6.94-7.09 (3H, m), 7.38 (1H, dd, J=6.9, 1.8 Hz), 9.8 (1H, br s).

### Reference Example 69

### 1-(2-aminopyridin-4-yl)-3-tert-butyl-1,3-dihydro-2H-benzimidazol-2-one

In the same manner as in Reference Example 42 and using 1-tert-butyl-1,3-dihydro-2H-benzimidazol-2-one obtained in Reference Example 68, the title compound was obtained as crystals. yield 86%.

melting point 177-178° C. (ethyl acetate-hexane)

¹H-NMR (CDCl₃) δ: 1.83 (9H, s), 4.57 (2H, br s), 6.76 (1H, s), 6.84 (1H, dd, J=5.7, 1.8 Hz), 7.01-7.12 (2H, m), 7.16-7.21 (1H, m), 7.43-7.48 (1H, m), 8.19 (1H, d, J=5.7 Hz).

### Reference Example 70

### 6-methyl-1,3-benzoxazol-2(3H)-one

In the same manner as in Reference Example 41 and using 2-amino-5-methylphenol, the title compound was obtained as crystals. yield 74%.

melting point 144-145° C. (ethyl acetate-hexane)

¹H-NMR (CDCl₃) δ: 2.39 (3H, s), 6.83-7.10 (3H, m), 9.44 (1H, br s).

### Reference Example 71

### 3-(2-aminopyridin-4-yl)-1,3-benzoxazol-2(3H)-one

In the same manner as in Reference Example 42 and using 1,3-benzoxazol-2(3H)-one, the title compound was obtained as crystals. yield 40%.

melting point 202-203° C. (ethyl acetate)

¹H-NMR (DMSO-d₆) δ: 6.20-7.38 (8H, m), 7.44 (1H, d, J=8.4 Hz).

### Reference Example 72

### 5-methyl-1,3-benzoxazol-2(3H)-one

In the same manner as in Reference Example 41 and using 2-amino-4-methylphenol, the title compound was obtained as crystals. yield 73%.

melting point 129-130° C. (ethyl acetate-hexane)

¹H-NMR (CDCl₃) δ: 2.38 (3H, s), 6.88-7.00 (2H, m), 7.08 (1H, d, J=8.1 Hz), 9.60 (1H, brs).

### Reference Example 73

### 3-(2-aminopyridin-4-yl)-6-methyl-1,3-benzoxazol-2(3H)-one

In the same manner as in Reference Example 42 and using 6-methyl-1,3-benzoxazol-2(3H)-one obtained in Reference Example 70, the title compound was obtained. yield 29%.

¹H-NMR (CDCl₃) δ: 2.42 (3H, s), 4.64 (2H, brs), 6.82 (1H, s), 6.91 (1H, dd, J=5.4, 1.8 Hz), 7.02 (1H, d, J=7.2 Hz), 7.10-7.17 (2H, m), 8.23 (1H, d, J=5.4 Hz).

### Reference Example 74

### 3-(2-aminopyridin-4-yl)-5-methyl-1,3-benzoxazol-2(3H)-one

In the same manner as in Reference Example 42 and using 5-methyl-1,3-benzoxazol-2(3H)-one obtained in Reference Example 72, the title compound was obtained as crystals. yield 25%.

melting point 173-174° C.

¹H-NMR (CDCl₃) δ: 2.40 (3H, s), 4.67 (2H, br s), 6.80 (1H, d, J=1.8 Hz), 6.90 (1H, dd, J=5.4, 1.8 Hz), 7.00 (1H, d, J=8.1 Hz), 7.06 (1H, s), 7.16 (1H, d, J=8.4 Hz), 8.23 (1H, d, J=5.4 Hz).

### Reference Example 75

### 1-(2-aminopyridin-4-yl)-3,4-dihydroquinolin-2(1H)-one

In the same manner as in Reference Example 42 and using 3,4-dihydroquinolin-2(1H)-one, the title compound was obtained as crystals. yield 46%.

melting point 204-205° C.

¹H-NMR (CDCl₃) δ: 2.75-2.83 (2H, s), 3.00-3.07 (2H, s), 4.59 (2H, s), 6.43 (1H, d, J=1.5 Hz), 6.48-6.55 (2H, m), 7.00-7.13 (2H, m), 7.21 (1H, dd, J=7.5, 0.9 Hz), 8.18 (1H, d, J=5.4 Hz).

### Reference Example 76

### 4-(2-aminopyridin-4-yl)-2H-1,4-benzoxazin-3(4H)-one

In the same manner as in Reference Example 42 and using 2H-1,4-benzoxazin-3(4H)-one, the title compound was obtained as crystals. yield 46%.

melting point 189-190° C.

¹H-NMR (CDCl₃) δ: 4.62 (2H, br s), 4.73 (2H, s), 6.48 (1H, s), 6.56-6.62 (2H, m), 6.88-7.09 (3H, m), 8.23 (1H, d, J=5.4 Hz).

### Reference Example 77

### N-(2-aminopyridin-4-yl)benzamide

In the same manner as in Reference Example 42 and using benzamide, the title compound was obtained as crystals. yield 97%.

melting point 187-188° C. (ethyl acetate-hexane)

¹H-NMR (CDCl₃) δ: 4.48 (2H, br s), 6.62 (1H, dd, J=5.4, 1.8 Hz), 7.26(1H, d, J=1.8 Hz), 7.47-7.62 (3H, m), 7.76 (1H,br s), 7.85 (2H, d, J=8.4 Hz), 8.01 (1H, d, J=6.0 Hz).

### Reference Example 78

### N-(2-aminopyridin-4-yl)pyridine-2-carboxamide

In the same manner as in Reference Example 42 and using pyridine-2-carboxamide, the title compound was obtained. yield 46%.

¹H-NMR (CDCl₃) δ: 4.53 (2H, br s), 6.78(1H, d, J=5.1 Hz), 7.30 (1H, br s), 7.51 (1H, dd, J=7.2, 5.1 Hz), 7.92 (1H, dt, J=7.5, 1.5 Hz), 8.01 (1H, d, J=4.8 Hz), 8.27(1H, d, J=7.8 Hz), 8.61 (1H, dd, J=5.1, 1.5 Hz), 10.0 (1H, s).

### Reference Example 79

### N-(2-aminopyridin-4-yl)-2-phenylacetamide

In the same manner as in Reference Example 42 and using 2-phenylacetamide, the title compound was obtained. yield 89%.

¹H-NMR (CDCl₃) δ: 3.73 (2H, s), 4.41 (2H, br s), 6.35 (1H, dd, J=5.7, 1.5 Hz), 7.01 (1H, s), 7.09 (1H, br s), 7.25-7.43 (5H, m), 7.88 (1H, d, J=6.0 Hz).

### Reference Example 80

### N-benzyl-N′-(5-bromopyridin-2-yl)urea

In the same manner as in Example 1 and using 2-amino-5-bromopyridine, the title compound was obtained. yield 79%.

¹H-NMR (CDCl₃) δ: 4.61 (2H, d, J=6.0 Hz), 6.81 (1H, d, J=8.8 Hz), 7.22-7.39 (5H, m), 7.62 (1H, dd, J=8.8, 2.5 Hz), 8.17 (1H, d, J=2.5 Hz), 9.51 (1H, br s), 9.70 (1H, br s).

### Reference Example 81

### 1-(6-aminopyridin-3-yl)pyrrolidin-2-one

A solution of palladium acetate (0.22 g, 1.0 mmol) and 5-bis(diphenylphosphino)-9,9-dimethylxanthine (0.58 g, 1.0 mmol) in toluene (30 mL) was stirred at room temperature for 15 min under an argon atmosphere. 5-Bromo-2-nitropyridine (1.0 g, 5.0 mmol), 2-pyrrolidone (0.64 g, 7.5 mmol), copper iodide(I) (0.19 g, 1.0 mmol) and cesium carbonate (2.4 g, 7.5 mmol) were added to the mixture, and the mixture was stirred at 75° C. for 24 hr. The reaction solution was filtered through glass filter (6.7 cmφ) in which silica gel (30 g) and ethyl acetate were charged to remove the insoluble material. The solvent was evaporated under reduced pressure, and the precipitated crystals were collected by filtration to give 1-(6-nitropyridin-3-yl)pyrrolidin-2-one (0.45 g, yield 44%) as pale-yellow crystals.

¹H-NMR (400 MHz, CDCl₃) δ: 2.30 (2H, dt, J=15.2, 7.7 Hz), 2.71 (2H, t, J=8.2 Hz), 3.99 (2H, t, J=7.0 Hz), 8.30 (1H, d, J=8.8 Hz), 8.67 (1H, dd, J=8.8, 2.4 Hz), 8.72 (1H, d, J=2.7 Hz)

1-(6-Nitropyridin-3-yl)pyrrolidin-2-one (2.3 g, 11 mmol) was dissolved in ethanol (220 mL), reduced iron (3.0 g, 54 mmol) and aqueous calcium chloride solution (0.60 g, 5.4 mmol, water 23 mL) were added, and the mixture was heated at 80° C. for 2 hr. The reaction mixture was concentrated under reduced pressure, and the residue was dissolved in a small amount of hot methanol, and purified by NH type-silica gel chromatography. The object fraction was concentrated to give the title compound (1.3 g, yield 68%) as a solid.

¹H-NMR (400 MHz, CD₃OD) δ: 2.17 (2H, dt, J=15.3, 7.6, 7.5 Hz), 2.54 (2H, t, J=8.1 Hz), 3.83 (2H, t, J=7.1 Hz), 6.60 (1H, d, J=9.0 Hz), 7.69 (1H, dd, J=8.8, 2.7 Hz), 8.04 (1H, d, J=2.7 Hz)

### Reference Example 82

### 1-(2-aminopyridin-3-yl)pyrrolidin-2-one

A solution of palladium acetate (1.1 g, 4.9 mmol) and 5-bis(diphenylphosphino)-9,9-dimethylxanthine (2.9 g, 4.9 mmol) in toluene (150 mL) was stirred at room temperature for 15 min under an argon atmosphere. 3-Bromo-2-nitropyridine (5.0 g, 25 mmol), 2-pyrrolidone (2.5 g, 30 mmol), copper iodide(I) (0.94 g, 4.9 mmol) and cesium carbonate (12 g, 37 mmol) were added to the mixture, and the mixture was stirred at 75° C. for 3 hr. Ethyl acetate (200 mL) was added, and the insoluble material was removed using filter paper. The solvent was evaporated under reduced pressure. The residue was purified by silica gel chromatography, the object fraction was concentrated, and the residue was purified by NH type-silica gel chromatography to give 1-(2-nitropyridin-3-yl)pyrrolidin-2-one (2.7 g) as an oil.

The obtained 1-(2-nitropyridin-3-yl)pyrrolidin-2-one (2.7 g, 13 mmol) was dissolved in ethanol (140 mL), reduced iron (3.6 g, 65 mmol) and aqueous calcium chloride solution (0.71 g, 6.4 mmol, water 14 mL) were added, and the mixture was heated at 80° C. for 2 hr. Ethyl acetate (200 mL) was added, and the insoluble material was removed using filter paper. The solvent was evaporated under reduced pressure. The residue was purified by NH type-silica gel chromatography, and the object fraction was concentrated. The obtained residue was recrystallized from ethyl acetate to give the title compound (0.74 g, yield 32%).

¹H-NMR (400 MHz, DMSO-d₆) δ: 2.10 (2H, dt, J=15.2, 7.5, 7.3 Hz), 2.38 (2H, t, J=8.1 Hz), 3.57 (2H, t, J=7.1 Hz), 5.87 (2H, s), 6.57 (1H, dd, J=7.6, 4.9 Hz), 7.31 (1H, dd, J=7.6, 1.7 Hz), 7.89 (1H, dd, J=4.9, 1.7 Hz)

### Example 1

### N-benzyl-N′-(4-(2-oxopyrrolidin-1-yl)pyridin-2-yl)urea

A solution (3 ml) of 1-(2-aminopyridin-4-yl)pyrrolidin-2-one (106 mg, 0.6 mmol) obtained in Reference Example 3 and benzyl isocyanate (74 μl, 0.6 mmol) in tetrahydrofuran was stirred at 80° C. for 6 hr. The mixture was filtered, and washed with ethanol to give the title compound. yield 66%.

melting point 214-215° C. (tetrahydrofuran)

¹H-NMR (DMSO-d₆) δ: 2.06 (2H, tt, J=8.0, 6.9 Hz), 2.53 (2H, J=8.0 Hz), 3.77 (2H, t, J=6.9 Hz), 4.40 (2H, d, J=6.0 Hz), 7.24-7.40 (6H m), 7.57 (1H, s), 8.08 (1H, d, J=6.0 Hz), 8.70 (1H, brs), 9.27 (1H, brs).

### Example 2

### N-ethyl-N′-(4-(2-oxopyrrolidin-1-yl)pyridin-2-yl)urea

In the same manner as in Example 1 and using ethyl isocyanate, the title compound was obtained. yield 40%.

melting point 171-173° C. (tetrahydrofuran)

¹H-NMR (DMSO-d₆) δ: 1.08 (2H, t, J=6.9 Hz), 2.06 (2H, tt, J=6.9, 6.3 Hz), 2.52 (2H, q, J=6.9 Hz), 3.18 (2H, t, J=6.3 Hz), 3.77 (2H, t, J=6.9 Hz), 7.39 (1H, dd, J=5.8, 1.6 Hz), 7.52 (1H, d, J=1.6 Hz), 8.08 (1H, d, J=5.8 Hz), 8.22 (1H, brs), 9.11 (1H, brs).

### Example 3

### N-benzyl-N′-(4-(2-oxo-4-phenylpyrrolidin-1-yl)pyridin-2-yl)urea

In the same manner as in Example 1 and using 1-(2-aminopyridin-4-yl)-4-phenylpyrrolidin-2-one obtained in Reference Example 4, the title compound was obtained. yield 57%.

melting point 165-166° C. (tetrahydrofuran)

¹H-NMR (DMSO-d₆) δ: 2.79 (1H, dd, J=17.1, 9.9 Hz), 2.91 (1H, dd, J=17.1, 7.7 Hz), 3.64-3.81 (2H, m), 4.16 (1H, t, J=7.7 Hz), 4.38 (2H, d, J=6.0 Hz), 7.16-7.42 (1H, m), 7.62 (1H, d, J=1.6 Hz), 8.07 (1H, d, J=6.0 Hz), 8.64 (1H, brs), 9.24 (1H, brs).

### Example 4

### N-(4-(2-oxo-4-phenylpyrrolidin-1-yl)pyridin-2-yl)-N′-(pyridin-2-ylmethyl)urea

To a solution of 1-(2-aminopyridin-4-yl)-4-phenylpyrrolidin-2-one (532 mg, 3 mmol) obtained in Reference Example 4 and triethylamine (0.26 ml, 3.6 mmol) in tetrahydrofuran (20 ml) was added 2,2,2-trichloroethyl chloroformate (0.5 ml, 3.6 mmol) at 0° C., and the mixture was stirred for 10 min. The mixture was diluted with water, and extracted with ethyl acetate. The extract was washed with water, and dried over anhydrous magnesium sulfate. A solution of the obtained residue, diisopropylethylamine (0.63 ml, 3.6 mmol) and 1-(pyridin-2-yl)methanamine (0.30 ml, 3 mmol) in DMSO (3 ml) was stirred at 70° C. for 3 hr. The mixture was diluted with water, and extracted with ethyl acetate. The extract was washed with water, and dried over anhydrous magnesium sulfate. The solvent was evaporated under reduced pressure, and the obtained residue was purified by silica gel column chromatography (ethyl acetate) to give the title compound (342 mg, yield 30%) as crystals.

melting point 152-153° C. (ethanol)

¹H-NMR (DMSO-d₆) δ: 2.80 (1H, dd, J=16.5, 9.6 Hz), 2.92 (1H, dd, J=16.5, 8.3 Hz), 3.65-3.83 (2H, m), 4.17 (1H, t, J=6.9 Hz), 4.48 (2H, d, J=5.5 Hz), 7.21-7.43 (7H, m), 7.64 (1H, d, J=1.6 Hz), 7.75 (1H, td, J=7.5, 1.6 Hz), 8.11 (1H, d, J=5.8 Hz), 8.51 (1H, d, J=5.8 Hz), 8.81 (1H, brs).

### Example 5

### (−)-N-(4-(2-oxo-4-phenylpyrrolidin-1-yl)pyridin-2-yl)-N′-(pyridin-2-ylmethyl)urea

A compound having a shorter retention time was obtained from N-(4-(2-oxo-4-phenylpyrrolidin-1-yl)pyridin-2-yl)-N′-(pyridin-2-ylmethyl)urea synthesized in Example 4 using high performance liquid chromatography (apparatus: Waters semi-preparative system, column: CHIRALCEL OJ (50(i,d)x 500 mm) manufactured by DIACEL CHEMICAL INDUSTRIES, LTD.), mobile phase: hexane:ethanol:diethylamine=50:50:0.1, flow rate: 75 mL/min, column temperature: 35° C., injection volume: 75 mg).

melting point 150-151° C.

[α]_{D}²⁰=−13.2° (c=0.5090, chloroform)

### Example 6

### (+)-N-(4-(2-oxo-4-phenylpyrrolidin-1-yl)pyridin-2-yl)-N′-(pyridin-2-ylmethyl)urea

A compound having a longer retention time was obtained from N-(4-(2-oxo-4-phenylpyrrolidin-1-yl)pyridin-2-yl)-N′-(pyridin-2-ylmethyl)urea synthesized in Example 4 using high performance liquid chromatography (apparatus: Waters semi-preparative system, column: CHIRALCEL OJ (50(i,d)x 500 mm) manufactured by DIACEL CHEMICAL INDUSTRIES, LTD.), mobile phase: hexane:ethanol:diethylamine=50:50:0.1, flow rate: 75 mL/min, column temperature: 35° C., injection volume: 75 mg).

melting point 151-153° C.

[α]_{D}²⁰=+14.5° (c=0.5195, chloroform)

### Example 7

### N-(4-(2-oxo-4-phenylpyrrolidin-1-yl)pyridin-2-yl)-N′-(pyridin-3-ylmethyl)urea

In the same manner as in Example 4, and using 1-(2-aminopyridin-4-yl)-4-phenylpyrrolidin-2-one obtained in Reference Example 4 and 1-(pyridin-3-yl)methanamine, the title compound was obtained. yield 77%.

melting point 179-180° C. (tetrahydrofuran)

¹H-NMR (DMSO-d₆) δ: 2.81 (1H, dd, J=16.5, 9.6 Hz), 2.91 (1H, dd, J=16.5, 7.7 Hz), 3.63-3.84 (2H m), 4.17(1H, d, J=6.9 Hz), 4.41 (2H, d, J=5.8 Hz), 7.18-7.44 (7H, m), 7.63 (1H, d, J=1.6 Hz), 7.70 (1H, d, J=8.80 Hz), 8.10 (1H, d, J=5.8 Hz), 8.44 (1H, dd, J=4.7, 1.6 Hz), 8.52 (1H, d, J=1.6 Hz), 8.74 (1H, brs), 9.31 (1H, brs).

### Example 8

### N-(4-(2-oxo-4-phenylpyrrolidin-1-yl)pyridin-2-yl)-N′-(pyridin-4-ylmethyl)urea

In the same manner as in Example 4, and using 1-(2-aminopyridin-4-yl)-4-phenylpyrrolidin-2-one obtained in Reference Example 4 and 1-(pyridin-4-yl)methanamine, the title compound was obtained. yield 78%.

melting point 174-176° C. (tetrahydrofuran)

¹H-NMR (DMSO-d₆) δ: 2.70-3.00 (2H, m), 3.59-3.85 (2H, m), 4.06-4.23 (1H, m), 4.41 (2H, d, J=6.0 Hz), 7.25 (3H, t, J=5.8 Hz), 7.30-7.46 (5H, m), 7.62 (1H, s), 8.10 (1H, d, J=5.8 Hz), 8.48 (2H, d, J=5.2 Hz), 8.79 (1H, s), 9.35 (1H, s).

### Example 9

### N-((1-methylpiperidin-2-yl)methyl)-N′-(4-(2-oxo-4-phenylpyrrolidin-1-yl)pyridin-2-yl)urea

In the same manner as in Example 4, and using 1-(2-aminopyridin-4-yl)-4-phenylpyrrolidin-2-one obtained in Reference Example 4 and 1-(1-methylpiperidin-2-yl)methanamine, the title compound was obtained. yield 70%.

melting point 144-146° C. (tetrahydrofuran)

¹H-NMR (DMSO-d₆) δ: 1.10-1.72 (5H, m), 1.83-2.02 (2H, m), 2.17 (3H, s), 2.68-2.96 (3H, m), 3.13-3.30 (2H, m), 3.66-3.80 (2H, m), 4.08-4.22 (1H, m), 7.20-7.43 (6H, m), 8.12 (1H, brs), 9.15 (1H, brs).

### Example 10

### N-(2-furylmethyl)-N′-(4-(2-oxo-4-phenylpyrrolidin-1-yl)pyridin-2-yl)urea

In the same manner as in Example 4, and using 1-(2-aminopyridin-4-yl)-4-phenylpyrrolidin-2-one obtained in Reference Example 4 and 1-(2-furyl)methanamine, the title compound was obtained. yield 95%.

melting point 158-160° C. (tetrahydrofuran)

¹H-NMR (DMSO-d₆) δ: 2.72-3.00 (2H, m), 3.63-3.84 (2H, m), 4.17 (1H, t, J=7.0 Hz), 4.37 (2H, d, J=5.5 Hz), 6.27 (1H, d, J=2.5 Hz), 6.34-6.46 (1H, m), 7.22-7.51 (6H, m), 7.59 (1H, s), 7.64 (1H, s), 8.10 (1H, d, J=6.0 Hz), 8.58 (1H, brs), 9.26 (1H, s).

### Example 11

### N-(4-(2-oxo-4-phenylpyrrolidin-1-yl)pyridin-2-yl)-N′-(2-thienylmethyl)urea

In the same manner as in Example 4, and using 1-(2-aminopyridin-4-yl)-4-phenylpyrrolidin-2-one obtained in Reference Example 4 and 1-(2-thienyl)methanamine, the title compound was obtained. yield 60%.

melting point 179-180° C. (tetrahydrofuran)

¹H-NMR (DMSO-d₆) δ: 2.79 (1H, dd, J=17.0, 9.1 Hz), 3.00 (1H, dd, J=17.0, 8.5 Hz), 3.66 (1H, m), 3.75-3.83 (1H, m), 4.13 (1H, dd, J=9.1, 8.5 Hz), 4.43 (2H, d, J=5.8 Hz), 6.75 (1H, dd, J=5.9, 2.1 Hz), 6.86 (2H, d, J=8.8 Hz), 6.95 (1H, d, J=1.4 Hz), 7.23-7.43 (7H, m), 8.04 (1H, d, J=6.0 Hz).

### Example 12

### N-(4-(2-oxo-4-phenylpyrrolidin-1-yl)pyridin-2-yl)-N′-(tetrahydrofuran-2-ylmethyl)urea

In the same manner as in Example 4, and using 1-(2-aminopyridin-4-yl)-4-phenylpyrrolidin-2-one obtained in Reference Example 4 and 1-(tetrahydrofuran-2-yl)methanamine, the title compound was obtained. yield 98%.

melting point 170-171° C. (tetrahydrofuran)

¹H-NMR (DMSO-d₆) δ: 1.43-1.59 (1H, m), 1.73-1.97 (3H, m), 2.80 (1H, dd, J=16.8, 9.9 Hz), 2.91 (1H, dd, J=16.8, 8.0 Hz), 3.19 (1H, t, J=6.0 Hz), 3.25-3.31 (1H, m), 3.56-3.68 (1H, m), 3.68-3.83 (3H, m), 3.83-3.96 (1H, m), 4.16 (1H, t, J=6.6 Hz), 7.22-7.32 (1H, m), 7.32-7.44 (5H, m), 7.60 (1H, d, J=1.1 Hz), 8.09 (1H, d, J=5.8 Hz), 8.34 (1H, s), 9.18 (1H, s).

### Example 13

### N-(1,3-benzodioxol-5-ylmethyl)-N′-(4-(2-oxo-4-phenylpyrrolidin-1-yl)pyridin-2-yl)urea

In the same manner as in Example 4, and using 1-(2-aminopyridin-4-yl)-4-phenylpyrrolidin-2-one obtained in Reference Example 4 and 1-(1,3-benzodioxol-5-yl)methanamine, the title compound was obtained. yield 57%.

melting point 185-188° C. (tetrahydrofuran)

¹H-NMR (DMSO-d₆) δ: 2.68-2.98 (2H, m), 3.64-3.84 (2H, m), 4.08-4.19 (1H, m), 4.28 (2H, d, J=5.8 Hz), 5.94-5.99 (3H, m), 6.67-6.90 (4H, m), 7.22-7.31 (1H, m), 7.31 (1H, m), 7.31-7.44 (4H, m), 7.63 (1H, brs), 8.08 (1H, d, J=5.8 Hz), 8.57 (1H, brs).

### Example 14

### N-(4-(2-oxo-4-phenylpyrrolidin-1-yl)pyridin-2-yl)-N′-(2-pyridin-2-ylethyl)urea

In the same manner as in Example 4, and using 1-(2-aminopyridin-4-yl)-4-phenylpyrrolidin-2-one obtained in Reference Example 4 and 2-(pyridin-2-yl)ethanamine, the title compound was obtained. yield 63%.

melting point 202-204° C. (tetrahydrofuran)

¹H-NMR (DMSO-d₆) δ: 2.69-3.03 (5H, m), 3.45-3.63 (2H, m), 3.63-3.82 (2H, m), 7.18-7.49 (8H, m), 7.57 (1H, s), 7.71 (1H, td, J=7.7, 1.6 Hz), 8.01 (1H, d, J=6.0 Hz), 8.41 (1H, brs), 8.53 (1H, d, J=4.4 Hz), 9.17 (1H, brs).

### Example 15

### N-(2-isopropoxyethyl)-N′-(4-(2-oxo-4-phenylpyrrolidin-1-yl)pyridin-2-yl)urea

In the same manner as in Example 4, and using 1-(2-aminopyridin-4-yl) -4-phenylpyrrolidin-2-one obtained in Reference Example 4 and 2-isopropoxyethanamine, the title compound was obtained. yield 89%.

melting point 170-173° C. (tetrahydrofuran)

¹H-NMR (DMSO-d₆) δ: 1.90 (6H, d, J=6.3 Hz), 2.80 (1H, dd, J=16.5, 5.5 Hz), 2.92 (1H, dd, J=16.5, 5.5 Hz), 3.29 (2H, t, J=5.5 Hz), 3.44 (2H, t, J=5.5 Hz), 3.57 (1H, q, J=6.3 Hz), 3.66-3.83 (2H, m), 4.17 (1H, t, J=6.6 Hz), 7.23-7.45 (6H, m), 7.59 (1H, s), 8.08 (1H, d, J=5.8 Hz), 8.43 (1H, s), 9.22 (1H, s).

### Example 16

### N-(2-cyanoethyl)-N′-(4-(2-oxo-4-phenylpyrrolidin-1-yl)pyridin-2-yl)urea

In the same manner as in Example 4, and using 1-(2-aminopyridin-4-yl)-4-phenylpyrrolidin-2-one obtained in Reference Example 4 and 3-aminopropanenitrile, the title compound was obtained. yield 50%.

melting point 260-263° C. (tetrahydrofuran)

¹H-NMR (DMSO-d₆) δ: 2.66 (4H, m), 3.43 (2H, q, J=5.8 Hz), 3.67-3.82 (2H, m), 4.02-4.28 (1H, m), 7.64 (1H, s), 8.12 (1H, d, J=5.8 Hz), 8.56 (1H, brs), 9.33 (1H, brs).

### Example 17

### N-(2-((5-nitropyridin-2-yl)amino)ethyl)-N′-(4-(2-oxo-5-phenyl-1,3-oxazolidin-3-yl)pyridin-2-yl)urea

In the same manner as in Example 4, and using 3-(2-aminopyridin-4-yl)-5-phenyl-1,3-oxazolidin-2-one obtained in Reference Example 42 and N-(5-nitropyridin-2-yl)ethane-1,2-diamine, the title compound was obtained. yield 8%.

melting point 210-213° C. (ethanol)

¹H-NMR (DMSO-d₆) δ: 3.32-3.48 (2H, m), 3.41-3.65 (2H, m), 3.91 (1H, t, J=8.5 Hz), 4.44 (1H, t, J=8.8 Hz), 5.78 (1H, t, J=8.1 Hz), 6.57 (1H, d, J=8.8 Hz), 7.17 (1H, d, J=7.4 Hz), 7.37-7.58 (6H, m), 7.97-8.16 (2H, m), 8.18-8.31 (1H, m), 8.30-8.43 (1H, m), 8.85 (1H, d, J=2.5 Hz), 9.24 (1H, s).

### Example 18

### N-(4-(2-oxo-5-phenyl-3-oxazolidinyl)-2-pyridyl)-N′-(2-(2-pyridylthio)ethyl)urea

In the same manner as in Example 4, and using 3-(2-aminopyridin-4-yl)-5-phenyl-1,3-oxazolidin-2-one obtained in Reference Example 42 and 2-(2-pyridylthio)ethanamine hydrochloride, the title compound was obtained. yield 28%.

melting point 209-211° C. (ethanol)

¹H-NMR (DMSO-d₆) δ: 3.23-3.34 (2H, m), 3.44 (2H, q, J=6.2 Hz), 3.92 (1H, t, J=8.5 Hz), 4.44 (1H, t, J=8.9 Hz), 5.77 (1H, t, J=8.1 Hz), 7.08 (1H, dd, J=7.2, 4.7 Hz), 7.20 (1H, dd, J=5.9, 1.9 Hz), 7.32 (1H, d, J=8.0 Hz), 7.36-7.56 (6H, m), 7.61 (1H, td, J=7.2, 1.9 Hz), 8.06 (1H, d, J=5.8 Hz), 8.34-8.46 (2H, m), 9.22 (1H, s).

### Example 19

### N-(4-(2-oxopyrrolidin-1-yl)pyridin-2-yl)-N′-((pyridin-2-yl)methyl)urea

In the same manner as in Reference Example 3, and using N-(4-iodopyridin-2-yl)-N′-((pyridin-2-yl)methyl)urea obtained in Reference Example 27 and pyrrolidin-2-one, the title compound was obtained. yield 45%.

melting point 234-235° C. (tetrahydrofuran)

¹H-NMR (DMSO-d₆) δ: 2.06 (2H, m), 2.48-2.58(2H, m), 3.77 (2H, t, J=7.1 Hz), 4.48 (2H, d, J=5.8 Hz), 7.26 (1H, d, J=7.7, 4.9 Hz), 7.32 (1H, d, J=7.7 Hz), 7.39 (1H, dd, J=6.0, 1.9 Hz), 7.56 (1H, d, J=1.9 Hz), 7.76 (1H, td, J=7.7, 1.9 Hz), 8.51 (1H, d, J=4.9 Hz), 8.87 (1H, brs), 9.35 (1H, brs).

### Example 20

### bis(2,2,2-trichloroethyl)(4-(4-(4-methoxyphenyl)-2-oxopyrrolidin-1-yl)pyridin-2-yl)imidodicarbonate

A solution of 4-iodopyridin-2-amine (440 mg, 2 mmol), 4-(4-methoxyphenyl)pyrrolidin-2-one (459 mg, 2.4 mmol) obtained in Reference Example 8, potassium phosphate (552 mg, 4 mmol), copper iodide (76 mg, 0.4 mmol) and N,N′-dimethylethylenediamine (43 μl, 0.4 mmol) in dioxane (10 ml) was stirred at 100° C. for 14 hr under an argon atmosphere. Water was added to the mixture, and the mixture was extracted with ethyl acetate. The extract was washed with water, and dried over anhydrous sodium hydrogensulfate. The solvent was evaporated under reduced pressure. To a solution of the obtained residue and triethylamine (0.35 ml, 4.8 mmol) in tetrahydrofuran (20 ml) was added 2,2,2-trichloroethyl chloroformate (0.6 ml, 4.4 mmol) at 0° C., and the mixture was stirred for 10 min. The mixture was diluted with water, and extracted with ethyl acetate. The extract was washed with water, and dried over anhydrous magnesium sulfate. The obtained residue was purified by silica gel column chromatography (hexane:ethyl acetate=1:1) to give the title compound (100 mg, yield 15%) as a solid.

¹H-NMR (CDCl₃) δ: 2.75-2.87 (1H, m), 2.98-3.10 (1H. m), 3.65-3.77 (1H, m), 3.77-3.85 (1H, m), 3.82 (3H, s), 4.19 (1H, dd, J=9.3, 8.0 Hz), 4.79-4.83 (4H, m), 6.91 (1H, d, J=8.5 Hz), 7.19 (1H, d, J=8.5 Hz), 7.70 (1H, dd, J=5.8, 1.6 Hz), 7.73 (1H, d, J=1.6 Hz), 8.47 (1H, d, J=5.8 Hz).

### Example 21

### N-(4-(4-(4-methoxyphenyl)-2-oxopyrrolidin-1-yl)pyridin-2-yl)-N′-(pyridin-2-ylmethyl)urea

A solution of bis(2,2,2-trichloroethyl)(4-(4-(4-methoxyphenyl)-2-oxopyrrolidin-1-yl)pyridin-2-yl)imidodicarbonate (100 mg, 0.16 mmol) obtained in Example 20, diisopropylethylamine (0.06 ml, 0.35 mmol) and 1-(pyridin-2-yl)methanamine (0.03 ml, 0.35 mmol) in DMF (3 ml) was stirred at 70° C. for 8 hr. The mixture was diluted with water, and extracted with ethyl acetate. The extract was washed with water, and dried over anhydrous magnesium sulfate. The solvent was evaporated under reduced pressure, and the obtained residue was purified by silica gel column chromatography (ethyl acetate) to give the title compound (30 mg, yield 45%).

melting point 159-161° C. (ethanol)

¹H-NMR (DMSO-d₆) δ: 2.70-2.93 (2H, m), 3.59-3.71 (2H, m), 3.72 (3H, s), 4.11 (1H, t, J=7.8 Hz), 4.46 (2H, d, J=5.8 Hz), 6.90 (2H, d, J=8.5 Hz), 7.18-7.33 (4H, m), 7.37 (1H, dd, J=5.8, 1.9 Hz), 7.61 (1H, d, J=1.6 Hz), 7.74 (1H, t, J=7.7, 1.6 Hz), 8.09 (1H, d, J=5.8 Hz), 8.50 (1H, d, J=4.9 Hz), 8.81 (1H, brs), 9.32 (1H, s).

### Example 22

### bis(2,2,2-trichloroethyl)(4-(4-(3-methylphenyl)-2-oxopyrrolidin-1-yl)pyridin-2-yl)imidodicarbonate

In the same manner as in Example 20 and using 4-(3-methylphenyl)pyrrolidin-2-one obtained in Reference Example 11, the title compound was obtained. yield 48%.

¹H-NMR (CDCl₃) δ: 2.85 (1H, dd, J=17.3, 8.8 Hz), 3.06 (1H, dd, J=17.3, 8.8 Hz), 3.61-3.78 (1H, m), 3.86 (1H, dd, J=9.6, 7.4 Hz), 4.21 (1H, dd, J=9.6, 8.2 Hz), 4.78-4.84 (4H, m), 7.03-7.09 (2H, m), 7.13 (1H, d, J=7.7 Hz), 7.23-7.28 (1H, m), 7.70 (1H, dd, J=5.8, 1.9 Hz), 7.74 (1H, d, J=1.9 Hz), 8.48 (1H, d, J=5.8 Hz).

### Example 23

### N-(4-(4-(3-methylphenyl)-2-oxopyrrolidin-1-yl)pyridin-2-yl)-N′-((pyridin-2-yl)methyl)urea

In the same manner as in Example 21 and using bis(2,2,2-trichloroethyl)(4-(4-(3-methylphenyl)-2-oxopyrrolidin-1-yl)pyridin-2-yl)imidodicarbonate obtained in Example 22, the title compound was obtained. yield 45%.

melting point 186-187° C. (ethanol)

¹H-NMR (DMSO-d₆) δ: 2.30 (3H, s), 2.78 (1H, dd, J=17.0, 8.2 Hz), 2.90 (1H, dd, J=17.0, 8.2 Hz), 3.63-3.81 (2H, m), 4.15 (1H, t, J=8.1 Hz), 4.48 (2H, d, J=5.8 Hz), 7.09 (1H, d, J=7.7 Hz), 7.14-7.29 (4H, m), 7.33 (1H, d, J=7.7 Hz), 7.39 (1H, dd, J=5.8, 2.1 Hz), 7.64 (1H, d, J=1.6 Hz), 7.76 (1H, td, J=7.7, 1.6 Hz), 8.12 (1H, d, J=5.8 Hz), 8.52 (1H, d, J=4.11 Hz), 8.82 (1H, s), 9.34 (1H, s).

### Example 24

### bis(2,2,2-trichloroethyl)(4-(2-oxo-4-(3-(trifluoromethyl)phenyl)pyrrolidin-1-yl)pyridin-2-yl)imidodicarbonate

In the same manner as in Example 20 and using 4-(3-(trifluoromethyl)phenyl)pyrrolidin-2-one obtained in Reference Example 14, the title compound was obtained. yield 80%.

¹H-NMR (CDCl₃) δ: 2.86 (1H, dd, J=17.3, 8.5 Hz), 3.12 (1H, dd, J=17.3, 8.5 Hz), 3.71-3.97 (2H, m), 4.21-4.32 (1H, m), 4.81 (4H, s), 7.41-7.63 (4H, m), 7.70 (1H, dd, J=5.8, 1.9 Hz), 7.74 (1H, d, J=1.9 Hz), 8.49 (1H, d, J=5.8 Hz).

### Example 25

### N-(4-(2-oxo-4-(3-(trifluoromethyl)phenyl)pyrrolidin-1-yl)pyridin-2-yl)-N′-(pyridin-2-ylmethyl)urea

In the same manner as in Example 21 and using bis(2,2,2-trichloroethyl)(4-(2-oxo-4-(3-(trifluoromethyl)phenyl)pyrrolidin-1-yl)pyridin-2-yl)imidodicarbonate obtained in Example 24, the title compound was obtained. yield 76%.

melting point 197-199° C. (ethanol)

¹H-NMR (DMSO-d₆) δ: 2.89 (3H, t, J=8.1 Hz), 3.72-3.90 (2H, m), 4.15-4.23 (1H, m), 4.46 (2H, d, J=5.5 Hz), 7.20-7.28 (1H, m), 7.31 (1H, d, J=7.7 Hz), 7.36 (1H, dd, J=6.0, 1.9 Hz), 7.56-7.68 (3H, m), 7.71-7.78 (2H, m), 7.80 (1H, s), 8.11 (1H, d, J=5.8 Hz), 8.50 (1H, d, J=3.8 Hz), 8.78 (1H, brs), 9.31 (1H, s).

### Example 26

### bis(2,2,2-trichloroethyl)(4-(4-(3-methoxyphenyl)-2-oxopyrrolidin-1-yl)pyridin-2-yl)imidodicarbonate

In the same manner as in Example 20 and using 4-(3-methoxyphenyl)pyrrolidin-2-one obtained in Reference Example 17, the title compound was obtained. yield 73%.

¹H-NMR (CDCl₃) δ: 2.84 (1H, dd, J=17.3, 8.4 Hz), 3.07 (1H, dd, J=17.3, 8.5 Hz), 3.65-3.79 (1H, m), 3.83 (3H, s), 3.84-3.90 (1H, m), 4.21 (1H, t, J=8.8 Hz), 4.81 (4H, s), 6.80 (1H, s), 6.82-6.89 (1H, m), 7.30 (1H, t, J=8.0 Hz), 7.70 (1H, dd, J=5.8, 1.9 Hz), 7.73 (1H, d, J=1.9 Hz), 8.48 (1H, d, J=5.8 Hz).

### Example 27

### N-(4-(4-(3-methoxyphenyl)-2-oxopyrrolidin-1-yl)pyridin-2-yl)-N′-(pyridin-2-ylmethyl)urea

In the same manner as in Example 21 and using bis(2,2,2-trichloroethyl)(4-(4-(3-methoxyphenyl)-2-oxopyrrolidin-1-yl)pyridin-2-yl)imidodicarbonate obtained in Example 26, the title compound was obtained. yield 41%.

melting point 189-190° C. (ethanol)

¹H-NMR (DMSO-d₆) δ: 2.89 (3H, t, J=8.1 Hz), 3.72-3.90 (2H, m), 4.15-4.23 (1H, m), 4.46 (2H, d, J=5.5 Hz), 7.20-7.28 (1H, m), 7.31 (1H, d, J=7.7 Hz), 7.36 (1H, dd, J=6.0, 1.9 Hz), 7.56-7.68 (3H, m), 7.71-7.78 (2H, m), 7.80 (1H, s), 8.11 (1H, d, J=5.8 Hz), 8.50 (1H, d, J=3.8 Hz), 8.78 (1H, brs), 9.31 (1H, s).

### Example 28

### bis(2,2,2-trichloroethyl)(4-(4-(3-fluorophenyl)-2-oxopyrrolidin-1-yl)pyridin-2-yl)imidodicarbonate

In the same manner as in Example 20 and using 4-(3-fluorophenyl)pyrrolidin-2-one obtained in Reference Example 20, the title compound was obtained. yield 70%.

¹H-NMR (CDCl₃) δ: 2.76-2.89 (1H, m), 3.02-3.16 (1H, m), 3.66-3.81 (1H, m), 3.79-3.90 (1H, m), 4.23 (1H, dd, J=9.4, 8.0 Hz), 4.81 (4H, s), 6.93-7.09 (3H, m), 7.29-7.42 (1H, m), 7.70 (1H, dd, J=5.8, 1.9 Hz), 7.74 (1H, d, J=2.2 Hz), 8.49 (1H, d, J=5.8 Hz).

### Example 29

### N-(4-(4-(3-fluorophenyl)-2-oxopyrrolidin-1-yl)pyridin-2-yl)-N′-(pyridin-2-ylmethyl)urea

In the same manner as in Example 21 and using bis(2,2,2-trichloroethyl)(4-(4-(3-fluorophenyl)-2-oxopyrrolidin-1-yl)pyridin-2-yl)imidodicarbonate obtained in Example 28, the title compound was obtained. yield 78%.

melting point 188-189° C. (ethanol)

¹H-NMR (DMSO-d₆) δ: 2.78-2.92 (2H, m), 3.68-3.81 (1H, m), 4.09-4.22 (1H, m), 4.46 (1H, d, J=5.8 Hz), 7.09 (1H, td, J=8.7, 2.9 Hz), 7.19-7.44 (6H, m), 7.65 (1H, d, J=1.9 Hz), 7.74 (1H, td, J=7.6, 1.9 Hz), 8.10 (1H, d, J=5.8 Hz), 8.50 (1H, d, J=4.7 Hz), 8.78 (1H, brs), 9.31 (1H, s).

### Example 30

### bis(2,2,2-trichloroethyl)(4-(4-(4-fluorophenyl)-2-oxopyrrolidin-1-yl)pyridin-2-yl)imidodicarbonate

In the same manner as in Example 20 and using 4-(4-fluorophenyl)pyrrolidin-2-one obtained in Reference Example 23, the title compound was obtained. yield 38%.

¹H-NMR (CDCl₃) δ: 2.81 (1H, m), 3.05 (1H, dd, J=17.2, 8.7 Hz), 3.59-3.81 (1H, m), 3.79-3.94 (1H, m), 4.27 (1H, t, J=8.9 Hz), 4.87 (2H, s), 7.06 (2H, t, J=8.5 Hz), 7.26 (2H, t, J=6.7 Hz), 7.84-8.00 (2H, m), 8.45 (1H, d, J=5.8 Hz), 10.50 (1H, s).

### Example 31

### N-(4-(4-(4-fluorophenyl)-2-oxopyrrolidin-1-yl)pyridin-2-yl)-N′-(pyridin-2-ylmethyl)urea

In the same manner as in Example 21 and using bis(2,2,2-trichloroethyl)(4-(4-(4-fluorophenyl)-2-oxopyrrolidin-1-yl)pyridin-2-yl)imidodicarbonate obtained in Example 30, the title compound was obtained. yield 35%.

melting point 153-155° C. (ethanol)

¹H-NMR (DMSO-d₆) δ: 2.71-2.84 (1H, m), 2.84-2.95 (1H, m), 3.68-3.80 (2H, m), 4.07-4.21 (1H, m), 4.46 (1H, d, J=5.8 Hz), 7.17 (2H, t, J=8.8 Hz), 7.25 (1H, dd, J=7.3, 4.5 Hz), 7.31 (1H, d, J=7.7 Hz), 7.36 (1H,dd, J=5.8, 1.8 Hz), 7.43 (2H, dd, J=8.5, 5.5 Hz), 7.64 (1H, s), 7.74 (1H, td, J=7.6, 1.5 Hz), 8.10 (1H, d, J=5.8 Hz), 8.50 (1H, d, J=4.9 Hz), 8.77 (1H, brs), 9.31 (1H, s).

### Example 32

### bis(2,2,2-trichloroethyl)(4-(4-(2-fluorophenyl)-2-oxopyrrolidin-1-yl)pyridin-2-yl)imidodicarbonate

In the same manner as in Example 20 and using 4-(2-fluorophenyl)pyrrolidin-2-one obtained in Reference Example 26, the title compound was obtained. yield 47%.

¹H-NMR (CDCl₃) δ: 2.70-3.12 (2H, m), 3.62-4.04 (2H, m), 4.11-4.41 (1H, m), 4.74-4.85 (4H, m), 6.99-7.45 (4H, m, J=4.7 Hz), 7.87-7.93 (1H, m), 7.95 (1H, s), 8.45 (1H, dd, J=15.5, 5.9 Hz).

### Example 33

### N-(4-(4-(2-fluorophenyl)-2-oxopyrrolidin-1-yl)pyridin-2-yl)-N′-(pyridin-2-ylmethyl)urea

In the same manner as in Example 21 and using bis(2,2,2-trichloroethyl)(4-(4-(2-fluorophenyl)-2-oxopyrrolidin-1-yl)pyridin-2-yl)imidodicarbonate obtained in Example 32, the title compound was obtained. yield 78%.

melting point 173-174° C. (ethanol)

¹H-NMR (DMSO-d₆) δ: 2.73-2.85 (1H, m), 2.88-3.03 (1H, m), 3.68-3.85 (1H, m), 3.84-4.02 (1H, m), 4.10-4.24 (1H, m), 4.47 (2H, d, J=5.8 Hz), 7.15-7.41 (6H, m), 7.46 (1H, t, J=7.7 Hz), 7.64 (3H, s), 7.74 (1H, td, J=7.7, 1.9 Hz), 8.10 (1H, d, J=6.0 Hz), 8.50 (1H, d, J=4.7 Hz), 8.78 (1H, s), 9.32 (1H, s).

### Example 34

### N-(4-(4-benzyl-2-oxopyrrolidin-1-yl)pyridin-2-yl)-N′-((pyridin-2-yl)methyl)urea

In the same manner as in Reference Example 3, and using N-(4-iodopyridin-2-yl)-N′-((pyridin-2-yl)methyl)urea obtained in Reference Example 27 and 4-benzylpyrrolidin-2-one, the title compound was obtained. yield 30%.

melting point 215-220° C. (tetrahydrofuran)

¹H-NMR (DMSO-d₆) δ: 2.23-2.86 (5H, m), 3.50 (1H, dd, J=12.2, 5.1 Hz), 3.80 (1H, dd, J=8.6, 6.4 Hz), 4.48 (2H, d, J=5.8 Hz), 7.17-7.36 (7H, m), 7.37-7.45 (1H, m), 7.49 (1H, s), 7.77 (1H, td, J=7.6, 1.8 Hz), 8.09 (1H, d, J=5.8 Hz), 8.52 (1H, d, J=4.7 Hz), 8.86 (1H, brs), 9.34 (1H, brs).

### Example 35

### ethyl 3-benzyl-5-oxo-1-(2-((((pyridin-2-ylmethyl)amino)carbonyl)amino)pyridin-4-yl)pyrrolidine-3-carboxylate

In the same manner as in Example 4 and using ethyl 1-(2-aminopyridin-4-yl)-3-benzyl-5-oxopyrrolidine-3-carboxylate obtained in Reference Example 28, the title compound was obtained. yield 45%.

melting point 158-160° C.

¹H-NMR (DMSO-d₆) δ: 1.15 (3H, t, J=7.1 Hz), 2.72-2.95 (2H, m), 3.10 (2H, s), 3.87 (1H, d, J=10.2 Hz), 4.00-4.16 (3H, m), 4.47 (2H, d, J=5.5 Hz), 7.09-7.18 (1H, m), 7.19-7.37 (6H, m), 7.51 (1H, s), 7.75 (1H, td, J=7.7, 1.6 Hz), 8.08 (1H, d, J=6.0 Hz), 8.51 (1H, d, J=4.89 Hz), 8.75 (1H, brs), 9.30 (1H, s).

### Example 36

### bis(2,2,2-trichloroethyl)(4-(2-oxo-4-(2-phenylethyl)pyrrolidin-1-yl)pyridin-2-yl)imidodicarbonate

In the same manner as in Example 20 and using 4-(2-phenylethyl)pyrrolidin-2-one obtained in Reference Example 31, the title compound was obtained. yield 45%.

¹H-NMR (CDCl₃) δ: 1.80-1.98 (2H, m), 2.30-2.60 (2H, m), 2.67-2.85 (3H, m), 3.48 (1H, dd, J=9.3, 7.1 Hz), 3.92 (1H, dd, J=9.3, 7.7 Hz), 4.81 (4H, s), 7.14-7.37 (5H, m), 7.65 (1H, d, J=1.4 Hz), 7.69 (1H, dd, J=5.6, 2.1 Hz), 8.45 (1H, d, J=5.8 Hz).

### Example 37

### N-(4-(2-oxo-4-(2-phenylethyl)pyrrolidin-1-yl)pyridin-2-yl)-N′-(pyridin-2-ylmethyl)urea

In the same manner as in Example 21 and using bis(2,2,2-trichloroethyl)(4-(2-oxo-4-(2-phenylethyl)pyrrolidin-1-yl)pyridin-2-yl)imidodicarbonate obtained in Example 36, the title compound was obtained. yield 80%.

melting point 177° C. (ethanol)

¹H-NMR (DMSO-d₆) δ: 1.66-1.86 (2H, m), 2.28-2.46 (2H, m), 2.57-2.70 (3H, m), 3.48 (1H, dd, J=9.3, 7.1 Hz), 3.84-3.99 (1H, m), 4.49 (1H, d, J=5.8 Hz), 7.12-7.40 (7H, m), 7.61 (1H, s), 7.75 (1H, td, J=7.6, 1.5 Hz), 8.10 (1H, d, J=6.0 Hz), 8.51 (1H, d, J=4.4 Hz), 8.81 (1H, brs), 9.32 (1H, s).

### Example 38

### bis(2,2,2-trichloroethyl)(4-(2-oxo-4-(phenoxymethyl)pyrrolidin-1-yl)pyridin-2-yl)imidodicarbonate

In the same manner as in Example 20 and using 4-(phenoxymethyl)pyrrolidin-2-one obtained in Reference Example 35, the title compound was obtained. yield 44%.

¹H-NMR (CDCl₃) δ: 2.52-2.71 (1H, m), 2.78-2.99 (1H, m), 2.94-3.07 (1H, m), 3.61-4.20 (4H, m), 4.81 (4H, s), 6.86-6.92 (2H, m), 6.97 (1H, td, J=7.3, 1.1 Hz), 7.24-7.34 (2H, m).

### Example 39

### N-(4-(2-oxo-4-(phenoxymethyl)pyrrolidin-1-yl)pyridin-2-yl)-N′-(pyridin-2-ylmethyl)urea

In the same manner as in Example 21 and using bis(2,2,2-trichloroethyl)(4-(2-oxo-4-(phenoxymethyl)pyrrolidin-1-yl)pyridin-2-yl)imidodicarbonate obtained in Example 38, the title compound was obtained. yield 78%.

melting point 135-137° C. (ethanol)

¹H-NMR (DMSO-d₆) δ: 2.39-2.46 (1H, m), 2.67-2.83 (1H,m), 2.85-2.99 (1H, m), 3.31 (3H, s), 3.98 (1H, dd, J=9.8, 8.1 Hz), 4.04 (2H, d, J=6.6 Hz), 4.47 (2H, d, J=5.8 Hz), 6.84-7.00 (3H, m), 7.20-7.35 (4H, m), 7.45 (1H, dd, J=6.0, 1.9 Hz), 7.57 (1H, d, J=1.8 Hz), 7.74 (1H, td, J=7.6, 1.8 Hz), 8.09 (1H, d, J=6.0 Hz), 8.50 (1H, d, J=4.7 Hz), 8.77 (1H, brs), 9.31 (1H, s).

### Example 40

### bis(2,2,2-trichloroethyl)(4-(4-(2-(4-methoxyphenyl)ethyl)-2-oxopyrrolidin-1-yl)pyridin-2-yl)imidodicarbonate

In the same manner as in Example 20 and using 4-(2-(4-methoxyphenyl)ethyl)pyrrolidin-2-one obtained in Reference Example 40, the title compound was obtained. yield 48%.

¹H-NMR (CDCl₃) δ: 1.74-1.94 (2H, m), 2.29-2.55 (2H, m), 2.65 (2H, t, J=7.6 Hz), 2.70-2.82 (1H, m), 3.46 (1H, dd, J=8.8, 7.4 Hz), 3.78 (3H, s), 3.86-3.96 (1H, m), 4.80 (4H, s), 6.84 (2H, t, J=8.5 Hz), 7.09 (2H, t, J=8.5 Hz), 7.65 (1H, s), 7.68 (1H, dd, J=5.8, 1.6 Hz), 8.44 (1H, d, J=5.8 Hz).

### Example 41

### N-(4-(4-(2-(4-methoxyphenyl)ethyl)-2-oxopyrrolidin-1-yl)pyridin-2-yl)-N′-(pyridin-2-ylmethyl)urea

In the same manner as in Example 21 and using bis(2,2,2-trichloroethyl)(4-(4-(2-(4-methoxyphenyl)ethyl)-2-oxopyrrolidin-1-yl)pyridin-2-yl)imidodicarbonate obtained in Example 40, the title compound was obtained. yield 81%.

¹H-NMR (DMSO-d₆) δ: 1.65-1.81 (2H, m), 2.28-2.44 (2H, m), 2.56 (2H, t, J=7.7 Hz), 2.59-2.74 (1H, m), 3.47 (1H, dd, J=9.2, 6.7 Hz), 3.71 (3H, s), 3.89 (1H, dd, J=8.8, 7.7 Hz), 4.48 (2H, d, J=5.8 Hz), 6.84 (2H, d, J=8.5 Hz), 7.14 (2H, d, J=8.5 Hz), 7.26 (1H, dd, J=7.7, 4.9 Hz), 7.29-7.39 (2H, m), 7.60 (1H, s), 7.76 (1H, d, J=7.7, 1.6 Hz), 8.09 (1H, d, J=5.8 Hz), 8.51 (1H, d, J=4.9 Hz), 8.79 (1H, s), 9.31 (1H, s).

### Example 42

### N-(4-(2-oxo-5-phenyl-1,3-oxazolidin-3-yl)pyridin-2-yl)-N′-(pyridin-2-ylmethyl)urea

To a solution of 3-(2-aminopyridin-4-yl)-5-phenyl-1,3-oxazolidin-2-one (1.78 g, 6.97 mmol) obtained in Reference Example 42 and triethylamine (1.46 mL, 10.5 mmol) in tetrahydrofuran (50 ml) was added 2,2,2-trichloroethyl formate (1.45 ml, 1.21 mmol) at room temperature, and the mixture was stirred at room temperature for 2 hr. Water was added to the reaction mixture, the organic layer was separated, and the aqueous layer was extracted with ethyl acetate. The combined organic layers were washed with 1N hydrochloric acid and saturated aqueous sodium hydrogen carbonate solution, dried over anhydrous magnesium sulfate, filtered, and concentrated under reduced pressure to give a crude mixture of 2,2,2-trichloroethyl(4-(2-oxo-5-phenyl-1,3-oxazolidin-3-yl)pyridin-2-yl)carbamate and bis(2,2,2-trichloroethyl)(4-(2-oxo-5-phenyl-1,3-oxazolidin-3-yl)pyridin-2-yl)imidodicarbonate. To a solution of the mixture and 1-(pyridin-2-yl)methanamine (1.88 g, 17.4 mmol) in DMF (20 ml) was added diisopropylethylamine (3.0 ml, 17.4 mmol) at room temperature, and the mixture was stirred with heating at 60° C. for 6 hr. The reaction mixture was allowed to cool to room temperature, poured into water, and extracted with ethyl acetate. The organic layer was washed with saturated brine, dried over anhydrous sodium sulfate, and concentrated under reduced pressure. The obtained residue was purified by silica gel column chromatography (ethyl acetate) to give the title compound (785 mg, yield 29%) as crystals. melting point 190-191° C. (ethanol-hexane)

¹H-NMR (CDCl₃) δ: 3.88 (1H, dd, J=7.5, 9.0, Hz), 4.32 (1H, t, J=9.0 Hz), 4.70 (2H, d, J=5.7 Hz), 5.65 (1H, t, J=7.5 Hz), 6.99 (1H, s), 7.13 (1H, dd, J=7.5, 4.8 Hz), 7.26-7.50 (7H, m), 7.60 (1H, dt, J=7.5, 1.8 Hz), 8.14 (1H, d, J=6.0 Hz), 8.48-8.52 (1H, m), 8.59 (1H, s), 9.73 (1H, br s).

### Example 43

### N-(4-(5-benzyl-2-oxo-1,3-oxazolidin-3-yl)pyridin-2-yl)-N′-(pyridin-2-ylmethyl)urea

In the same manner as in Example 42 and using 3-(2-aminopyridin-4-yl)-5-benzyl-1,3-oxazolidin-2-one obtained in Reference Example 44, the title compound was obtained as crystals. yield 39%.

melting point 191-193° C. (ethanol-hexane)

¹H-NMR (CDCl₃) δ: 3.01 (1H, dd, J=13.8, 6.9 Hz), 3.19 (1H, dd, J=13.8, 6.0 Hz), 3.65 (1H, dd, J=8.7, 6.9 Hz), 3.92 (1H, t, J=8.7 Hz), 4.70 (2H, d, J=5.4 Hz), 4.83-4.95 (1H, m), 6.89 (1H, s), 7.15-7.40 (8H, m), 7.59-7.67 (1H, m), 8.09 (1H, d, J=6.0 Hz), 8.55 (1H, d, J=4.2 Hz), 8.60 (1H, s), 9.75 (1H, br s).

### Example 44

### N-(4-(2-oxo-5-(2-phenylethyl)-1,3-oxazolidin-3-yl)pyridin-2-yl)-N′-(pyridin-2-ylmethyl)urea

In the same manner as in Example 42 and using 3-(2-aminopyridin-4-yl)-5-(2-phenylethyl)-1,3-oxazolidin-2-one obtained in Reference Example 46, the title compound was obtained as crystals. yield 13%.

melting point 194-195° C. (ethyl acetate-hexane)

¹H-NMR (CDCl₃) δ: 1.92-2.25 (2H, m), 2.71-2.95 (2H, m), 3.54 (1H, t, J=8.4 Hz), 3.95 (1H, t, J=9.0 Hz), 4.57-4.72 (3H, m), 6.97 (1H, s), 6.92 (1H, s), 7.10-7.39 (8H, m), 7.62 (1H, t, J=7.8 Hz), 8.11 (1H, d, J=6.0 Hz), 8.51 (1H, d, J=4.5 Hz), 8.65 (1H, br s), 9.76 (1H, br s).

### Example 45

### N-(4-(2-oxo-5-(phenoxymethyl)-1,3-oxazolidin-3-yl)pyridin-2-yl)-N′-(pyridin-2-ylmethyl)urea

In the same manner as in Example 42 and using 3-(2-aminopyridin-4-yl)-5-(phenoxymethyl)-1,3-oxazolidin-2-one obtained in Reference Example 48, the title compound was obtained as crystals. yield 31%.

melting point 155-156° C. (ethyl acetate-hexane)

¹H-NMR (CDCl₃) δ: 3.96-4.26 (4H, m), 4.71 (2H, d, J=5.7 Hz), 4.94-5.08 (1H, m), 6.85-7.42 (9H, m), 7.61 (1H, dt, J=7.5, 1.8 Hz), 8.14 (1H, d, J=6.0 Hz), 8.54 (1H, d, J=3.3 Hz), 9.00 (1H, br s), 9.74 (1H, br s).

### Example 46

### N-(4-(5-((benzyloxy)methyl)-2-oxo-1,3-oxazolidin-3-yl)pyridin-2-yl)-N′-(pyridin-2-ylmethyl)urea

In the same manner as in Example 42 and using 3-(2-aminopyridin-4-yl)-5-((benzyloxy)methyl)-1,3-oxazolidin-2-one obtained in Reference Example 50, the title compound was obtained as crystals. yield 54%.

melting point 134-135° C. (ethyl acetate-hexane)

H-NMR (CDCl₃) δ: 3.63-3.76 (2H, m), 3.86 (1H, t, J=9.0 Hz), 3.96 (1H, t, J=9.0 Hz), 4.57 (1H, d, J=12.0 Hz), 4.62 (1H, d, J=12.0 Hz), 4.69-4.82 (3H, m), 6.97 (1H, s), 7.11-7.17 (1H, m), 7.20-7.32 (7H, m), 7.62 (1H, t, J=7.5 Hz), 8.12 (1H, d, J=6.0 Hz), 8.54 (1H, d, J=5.1 Hz), 8.60 (1H, br s), 9.76 (1H, br s).

### Example 47

### N-(4-(2-oxo-5-phenyl-1,3-oxazol-3(2H)-yl)pyridin-2-yl)-N′-(pyridin-2-ylmethyl)urea

To a solution of 3-(2-aminopyridin-4-yl)-5-phenyl-1,3-oxazol-2(3H)-one (230 mg, 0.91 mmol) obtained in Reference Example 51 and triethylamine (144 μl, 2.00 mmol) in tetrahydrofuran (7 ml) was added dropwise 2,2,2-trichloroethyl chlorocarbonate (275 μl, 2.00 mmol) at 0° C., and the mixture was stirred at room temperature for 2 hr. Water was added to the reaction mixture, and the mixture was extracted with ethyl acetate. The extract was washed with water and saturated brine, and dried over anhydrous magnesium sulfate. The solvent was evaporated under reduced pressure to give a solid (456 mg). 1-(Pyridin-2-yl)methanamine (167 μl, 1.65 mmol), diisopropylethylamine (287 μl, 1.65 mmol) and DMSO (5 ml) were added thereto, and the mixture was stirred at 70° C. for 6 hr. The reaction mixture was poured into water, and extracted with ethyl acetate. The extract was washed with water, and dried over anhydrous magnesium sulfate. The solvent was evaporated under reduced pressure. The residue was purified by preparative HPLC to give the title compound (79.0 mg, yield 27%) as a solid.

melting point 208-209° C. (ethyl acetate)

¹H-NMR (d₆-DMSO): δ 4.50 (2H, d, J=5.7 Hz), 7.25-7.32 (2H, m), 7.33-7.42 (2H, m), 7.49 (2H, t, J=7.5 Hz), 7.63-7.69 (2H, m), 7.78 (1H, td, J=7.7 Hz and 1.8 Hz), 8.13 (1H, d, J=1.9 Hz), 8.30 (1H, d, J=5.8 Hz), 8.35 (1H, s), 8.41-8.50 (1H, m), 8.51-8.56 (1H, m), 9.54 (1H, s).

### Example 48

### N-(4-(2-oxo-5-(2-thienyl)-1,3-oxazolidin-3-yl)pyridin-2-yl)-N′-(pyridin-2-ylmethyl)urea

In the same manner as in Example 42 and using 3-(2-aminopyridin-4-yl)-5-(2-thienyl)-1,3-oxazolidin-2-one obtained in Reference Example 52, the title compound was obtained as crystals. yield 42%.

melting point 187-188° C. (ethyl acetate-hexane)

¹H-NMR (CDCl₃) δ: 4.03 (1H, dd, J=9.0, 7.2 Hz), 4.32 (1H, t, J=9.0 Hz), 4.91 (2H, t, J=5.7 Hz), 5.88 (1H, t, J=7.5 Hz), 7.00-7.52 (7H, m), 7.62 (1H, dt, J=7.8, 1.8 Hz), 8.15 (1H, d, J=6.3 Hz), 8.52 (1H, d, J=3.9 Hz), 8.91 (1H, brs), 9. 75 (1H, brs).

### Example 49

### N-(4-(5-methyl-2-oxo-5-phenyl-1,3-oxazolidin-3-yl)pyridin-2-yl)-N′-(pyridin-2-ylmethyl)urea

In the same manner as in Example 42 and using 3-(2-aminopyridin-4-yl)-5-methyl-5-phenyl-1,3-oxazolidin-2-one obtained in Reference Example 57, the title compound was obtained as crystals. yield 67%.

melting point 194-195° C. (ethyl acetate-hexane)

¹H-NMR (CDCl₃) δ: 1.85 (3H, s), 4.06 (1H, d, J=9.0 Hz), 4.09 (1H, d, J=9.0 Hz), 4.71 (2H, d, J=5.7 Hz), 7.02 (1H, d, J=1.8 Hz), 7.15 (1H, dd, J=7.2, 5.4 Hz), 7.30-7.49 (7H, m), 7.62 (1H, dt, J=7.8, 1.8 Hz), 8.13 (1H, d, J=6.0 Hz), 8.53 (1H, d, J=4.2 Hz), 8.98 (1H, br s), 9.64 (1H, br s).

### Example 50

### bis(2,2,2-trichloroethyl)(4-(3-methyl-2-oxopyrrolidin-1-yl)pyridin-2-yl)imidodicarbonate

In the same manner as in Example 20 and using 3-methylpyrrolidin-2-one, the title compound was obtained. yield 45%.

¹H-NMR (CDCl₃) δ: 1.31 (3H, d, J=7.1 Hz), 1.73-1.92 (1H, m), 2.35-2.51 (1H, m), 2.63-2.83 (1H, m), 3.65-3.87 (2H, m), 4.81 (4H, s), 7.68 (1H, d, J=2.2 Hz), 7.75 (1H, dd, J=5.8, 2.2 Hz), 8.46 (1H, d, J=5.8 Hz).

### Example 51

### N-(4-(3-methyl-2-oxopyrrolidin-1-yl)pyridin-2-yl)-N′-((pyridin-2-yl)methyl)urea

In the same manner as in Example 21 and using bis(2,2,2-trichloroethyl)(4-(3-methyl-2-oxopyrrolidin-1-yl)pyridin-2-yl)imidodicarbonate obtained in Example 50, the title compound was obtained. yield 17%.

melting point 190-191° C. (ethanol)

¹H-NMR (DMSO-d₆) δ: 1.14 (3H, d, J=6.9 Hz), 1.53-1.80 (1H, m), 2.23-2.40 (1H, m), 2.57-2.79 (1H, m), 3.57-3.78 (1H, m), 4.47 (1H, d, J=5.5 Hz), 7.20-7.29 (1H, m), 7.31 (1H, d, J=8.0 Hz), 7.40 (1H, d, J=6.0 Hz), 7.57 (1H, s), 7.75 (1H, t, J=6.9 Hz), 8.08 (1H, d, J=6.0 Hz), 8.50 (1H, d, J=5.5 Hz), 8.83 (1H, s), 9.32 (1H, s).

### Example 52

### bis(2,2,2-trichloroethyl)(4-(2-methyl-5-oxopyrrolidin-1-yl)pyridin-2-yl)imidodicarbonate

In the same manner as in Example 20 and using 5-methylpyrrolidin-2-one, the title compound was obtained. yield 48%.

¹H-NMR (CDCl₃) δ: 1.33 (3H, d, J=6.0 Hz), 1.77-1.92 (1H, m)), 2.38 (1H, dd, J=12.6, 8.0 Hz), 2.49-2.64 (1H, m), 2.66-2.84 (1H, m), 4.41 (1H, dd, J=5.1 Hz and J=6.2 Hz), 4.71-4.87 (4H, m), 7.63 (1H, dd, J=5.5, 1.9 Hz), 7.70 (1H, d, J=1.9 Hz), 8.47 (1H, d, J=5.5 Hz).

### Example 53

### N-(4-(2-methyl-5-oxopyrrolidin-1-yl)pyridin-2-yl)-N′-((pyridin-2-yl)methyl)urea

In the same manner as in Example 21 and using bis(2,2,2-trichloroethyl)(4-(2-methyl-5-oxopyrrolidin-1-yl)pyridin-2-yl)imidodicarbonate obtained in Example 52, the title compound was obtained as a solid. yield 35%.

melting point 155-160° C. (ethanol)

¹H-NMR (DMSO-d₆) δ: 1.22 (3H, d, J=6.0 Hz), 1.62-1.78 (1H, m), 2.17-2.47 (2H, m), 2.60-2.76 (1H, m), 4.27-4.43 (1H, m), 4.48 (2H, d, J=5.8 Hz), 7.20-7.37 (3H, m), 7.56 (1H, s), 7.76 (1H, dt, J=7.7, 1.6 Hz), 8.11 (1H, d, J=5.8 Hz), 8.51 (1H, d, J=4.9 Hz), 8.81 (1H, brs), 9.34 (1H, s).

### Example 54

### N-(4-(3-oxo-2-azaspiro[4.5]dec-2-yl)pyridin-2-yl)-N′-(pyridin-2-ylmethyl)urea

In the same manner as in Reference Example 3, and using N-(4-iodopyridin-2-yl)-N′-((pyridin-2-yl)methyl)urea obtained in Reference Example 27 and 2-azaspiro[4.5]decan-3-one, the title compound was obtained. yield 15%.

melting point 191-192° C. (ethanol)

¹H-NMR (DMSO-d₆) δ: 1.26-1.57 (10H, m), 3.53 (2H, s), 4.47 (2H, d, J=5.8 Hz), 7.25 (1H, dd, J=6.9, 5.5 Hz), 7.31 (1H, d, J=7.7 Hz), 7.35 (1H, dd, J=6.1, 2.1 Hz), 7.61 (1H, d, J=1.6 Hz), 7.74 (1H, td, J=7.7, 1.6 Hz), 8.08 (1H, d, J=6.0 Hz), 8.50 (1H, d, J=4.1 Hz), 8.75 (1H, s), 9.29 (1H, s).

### Example 55

### bis(2,2,2-trichloroethyl)(4-(4,4-dimethyl-2-oxopyrrolidin-1-yl)pyridin-2-yl)imidodicarbonate

In the same manner as in Example 20 and using 4,4-dimethylpyrrolidin-2-one obtained in Reference Example 54, the title compound was obtained. yield 25%.

¹H-NMR (CDCl₃) δ: 1.25 (6H, s), 2.49 (2H, s), 3.55 (2H, s), 4.81 (4H, s), 7.66 (1H, d, J=2.2 Hz), 7.69 (1H, d, J=5.8, 2.2 Hz), 8.45 (1H, d, J=5.8 Hz).

### Example 56

### N-(4-(4,4-dimethyl-2-oxopyrrolidin-1-yl)pyridin-2-yl)-N′-(pyridin-2-ylmethyl)urea

In the same manner as in Example 21 and using bis(2,2,2-trichloroethyl)(4-(4,4-dimethyl-2-oxopyrrolidin-1-yl)pyridin-2-yl)imidodicarbonate obtained in Example 55, the title compound was obtained. yield 55%.

melting point 171-172° C. (ethanol)

¹H-NMR (DMSO-d₆) δ: 1.15 (6H, s), 2.40 (2H, s), 3.52 (2H, s), 4.48 (2H, d, J=5.8 Hz), 7.26 (1H, dd, J=7.0, 5.1 Hz), 7.32 (1H, d, J=7.7 Hz), 7.37 (1H, dd, J=5.8, 1.9 Hz), 7.58 (1H, d, J=1.9 Hz), 7.76 (1H, td, J=7.7, 1.9 Hz), 8.09 (1H, d, J=5.8 Hz), 8.51 (1H, d, J=4.1 Hz), 8.72 (1H, brs), 9.31 (1H, s).

### Example 57

### N-benzyl-N′-(4-(3,3-dimethyl-2-oxo-2,3-dihydro-1H-indol-1-yl)pyridin-2-yl)urea

In the same manner as in Example 1 and using 3,3-dimethyl-1,3-dihydro-2H-indol-2-one, the title compound was obtained. yield 60%.

melting point 182-183° C. (ethanol)

¹H-NMR (DMSO-d₆) δ: 1.40 (6H, s), 4.40 (2H, d, J=5.8 Hz), 7.05 (1H, d, J=8.0 Hz), 7.11 (1H, dd, J=5.5, 1.9 Hz), 7.16 (1H, td, J=7.4, 1.1 Hz), 7.20-7.39 (6H, m), 7.49 (1H, d, J=7.4 Hz), 7.67 (1H,d,J=1.4 Hz), 8.27 (1H, brs), 8.34 (1H, d, J=6.0 Hz), 9.45 (1H, brs).

### Example 58

### N-(4-(3,3-dimethyl-2-oxo-2,3-dihydro-1H-indol-1-yl)pyridin-2-yl)-N′-((pyridin-2-yl)methyl)urea

In the same manner as in Reference Example 3, and using 1-(4-iodopyridin-2-yl)-3-((pyridin-2-yl)methyl)urea obtained in Reference Example 27 and 3,3-dimethyl-1,3-dihydro-2H-indol-2-one, the title compound was obtained. yield 25%.

melting point 191-192° C. (ethanol)

¹H-NMR (DMSO-d₆) δ: 1.40 (6H, s), 4.49 (2H, d, J=5.8 Hz), 7.04 (1H, d, J=7.4 Hz), 7.11 (1H, dd, J=5.5, 1.1 Hz), 7.16 (1H, d, J=7.4 Hz), 7.27 (2H, t, J=7.2 Hz), 7.34 (1H, d, J=7.4 Hz), 7.48 (1H, d, J=7.2 Hz), 7.66 (1H, s), 7.77 (1H, td, J=7.7, 1.1 Hz), 8.36 (1H, d, J=5.5 Hz), 8.43 (1H, brs).

### Example 59

### N-(4-(3,3-dimethyl-2-oxo-2,3-dihydro-1H-pyrrolo[3,2-b]pyridin-1-yl)pyridin-2-yl)-N′-(pyridin-2-ylmethyl)urea

In the same manner as in Example 42 and using 1-(2-aminopyridin-4-yl)-3,3-dimethyl-1,3-dihydro-2H-pyrrolo[3,2-b]pyridin-2-one obtained in Reference Example 61, the title compound was obtained as crystals. yield 22%.

melting point 160-163° C. (ethyl acetate-hexane)

H-NMR (CDCl₃) δ: 1.54 (6H, s), 4.72 (2H, d, J=6.0 Hz), 7.07-7.19 (4H, m), 7.33-7.42 (2H, m), 7.63 (1H, t, J=7.5 Hz), 8.28-8.34 (2H, m), 8.56 (1H, d, J=4.8 Hz), 9.24 (1H, s), 9.72 (1H, br s).

### Example 60

### N-(4-(2′-oxospiro(cyclopentane-1,3′-indol)-1′(2′H)-yl)pyridin-2-yl)-N′-(pyridin-2-ylmethyl)urea

In the same manner as in Example 4 and using 1′-(2-aminopyridin-4-yl)spiro(cyclopentane-1,3′-indol)-2′(1′H)-one obtained in Reference Example 62, the title compound was obtained. yield 40%.

melting point 135° C.

¹H-NMR (DMSO-d₆) δ: 1.87-2.35 (8H, m), 4.73 (2H, d, J=5.8 Hz), 7.01-7.23 (6H, m), 7.24-7.30 (1H, m), 7.37 (1H, d, J=7.7 Hz), 7.63 (1H, td, J=7.7, 1.8 Hz), 8.31 (1H, d, J=5.5 Hz), 8.56 (1H, d, J=4.1 Hz), 8.61 (1H, brs), 9.82 (1H, brs).

### Example 61

### N-(4-(3-methyl-2-oxo-2,3-dihydro-1H-benzimidazol-1-yl)pyridin-2-yl)-N′-(pyridin-2-ylmethyl)urea

To a solution of 1-(2-aminopyridin-4-yl)-3-methyl-1,3-dihydro-2H-benzimidazol-2-one (350 mg, 1.46 mmol) obtained in Reference Example 64 and triethylamine (250 mg, 808 μmol) in tetrahydrofuran (20 ml) was added 2,2,2-trichloroethyl formate (296 mg, 1.21 mmol) at room temperature, and the mixture was stirred at room temperature for 2 hr. Water was added to the reaction mixture, the organic layer was separated, and the aqueous layer was extracted with ethyl acetate. The combined organic layers were washed with 1N hydrochloric acid and saturated aqueous sodium hydrogen carbonate solution, dried over anhydrous magnesium sulfate, filtered, and concentrated under reduced pressure to give a crude mixture of 2,2,2-trichloroethyl(4-(3-methyl-2-oxo-2,3-dihydro-1H-benzimidazol-1-yl)pyridin-2-yl)carbamate and bis(2,2,2-trichloroethyl)(4-(3-methyl-2-oxo-2,3-dihydro-1H-benzimidazol-1-yl)pyridin-2-yl)imidodicarbonate. To a solution of the mixture (400 mg, 850 mmol) and 1-(pyridin-2-yl)methanamine (275 mg, 2.55 mmol) in DMSO (20 mL) was added diisopropylethylamine (445 ml, 2.55 mmol) at room temperature, and the mixture was stirred with heating at 60° C. for 6 hr. The reaction mixture was allowed to cool to room temperature, poured into water, and extracted with chloroform. The organic layer was washed with saturated brine, dried over anhydrous sodium sulfate, and concentrated under reduced pressure. The obtained residue was crystallized from ethanol to give the title compound (196 mg, yield 28%) as crystals.

melting point 238-239° C. (ethanol)

¹H-NMR (DMSO-d₆) δ: 3.40 (3H, s), 4.50 (2H, d, J=5.7 Hz), 7.10-7.42 (7H, m), 7.73-7.78 (2H, m), 8.35 (1H, d, J=5.7 Hz), 8.56-8.62 (2H, m), 9.56 (1H, s).

### Example 62

### 1-(4-(3-phenyl-2-oxo-2,3-dihydro-1H-benzimidazol-1-yl)pyridin-2-yl)-3-(pyridin-2-ylmethyl)urea

In the same manner as in Example 61 and using 1-(2-aminopyridin-4-yl)-3-phenyl-1,3-dihydro-2H-benzimidazol-2-one obtained in Reference Example 66, the title compound was obtained as crystals. yield 17%.

melting point 231-232° C. (ethyl acetate-hexane)

¹H-NMR (CDCl₃) δ: 4.75 (2H, d, J=6.0 Hz), 7.07-7.72 (14H, m), 8.35 (1H, d, J=5.4 Hz), 8.56 (1H, d, J=3.9 Hz), 8.65 (1H, brs), 9.88 (1H, brs).

### Example 63

### N-(4-(3-tert-butyl-2-oxo-2,3-dihydro-1H-benzimidazol-1-yl)pyridin-2-yl)-N′-(pyridin-2-ylmethyl)urea

In the same manner as in Example 61 and using 1-(2-aminopyridin-4-yl)-3-tert-butyl-1,3-dihydro-2H-benzimidazol-2-one obtained in Reference Example 69, the title compound was obtained as crystals. yield 47%.

melting point 164-165° C. (ethyl acetate-hexane)

¹H-NMR (CDCl₃) δ: 1.83 (9H, s), 4.72 (2H, d, J=5.7 Hz), 6.98-7.30 (6H, m), 7.36 (1H, d, J=7.8 Hz), 7.47 (1H, d, J=8.4 Hz), 7.61 (1H, t, J=7.8 Hz), 8.31 (1H, d, J=6.0 Hz), 8.55 (1H, d, J=5.1 Hz), 8.85 (1H, s), 9.84 (1H, br s).

### Example 64

### N-(4-(2-oxo-1,3-benzoxazol-3(2H)-yl)pyridin-2-yl)-N′-(pyridin-2-ylmethyl)urea

In the same manner as in Example 61 and using 3-(2-aminopyridin-4-yl)-1,3-benzoxazol-2(3H)-one obtained in Reference Example 71, the title compound was obtained as a solid. yield 25%.

melting point 237-238° C. (ethyl acetate-hexane)

¹H-NMR (DMSO-d₆) δ: 4.50 (2H, d, J=6.0 Hz), 7.10-7.52 (7H, m), 7.70-7.80 (2H, m), 8.34-8.55 (3H, m), 9.61 (1H, s).

### Example 65

### 1-(4-(6-methyl-2-oxo-1,3-benzoxazol-3(2H)-yl)pyridin-2-yl)-3-(pyridin-2-ylmethyl)urea

In the same manner as in Example 61 and using 3-(2-aminopyridin-4-yl)-6-methyl-1,3-benzoxazol-2(3H)-one obtained in Reference Example 73, the title compound was obtained as a solid. yield 43%.

melting point 231-232° C. (ethyl acetate)

¹H-NMR (CDCl₃) δ: 2.40 (3H, s), 4.74 (2H, d, J=5.4 Hz), 6.93 (1H, d, J=8.4 Hz), 7.09-7.47 (6H, m), 7.62 (1H, t, J=7.5 Hz), 8.33 (1H, d, J=5.4 Hz), 8.55 (1H, d, J=5.1 Hz), 9.46 (1H, s), 9.79 (1H, br s).

### Example 66

### N-(4-(5-methyl-2-oxo-1,3-benzoxazol-3(2H)-yl)pyridin-2-yl)-N′-(pyridin-2-ylmethyl) urea

In the same manner as in Example 61 and using 3-(2-aminopyridin-4-yl)-5-methyl-1,3-benzoxazol-2(3H)-one obtained in Reference Example 74, the title compound was obtained as a solid. yield 48%.

melting point 205-206° C. (ethanol)

¹H-NMR (CDCl₃) δ: 2.39 (3H, s), 4.73 (2H, d, J=5.7 Hz), 7.02 (1H, d, J=9.3 Hz), 7.10 (1H, s), 7.15-7.26 (4H, m), 7.37 (1H, d, J=7.5 Hz), 7.64 (1H, dt, J=7.5, 1.8 Hz), 8.37 (1H, d, J=5.7 Hz), 8.54 (1H, s), 8.56 (1H, d, J=4.8 Hz), 9.74 (1H, s).

### Example 67

### 1-(4-(2-oxo-3,4-dihydroquinolin-1(2H)-yl)pyridin-2-yl)-3-(pyridin-2-ylmethyl)urea

In the same manner as in Example 61 and using 1-(2-aminopyridin-4-yl)-3,4-dihydroquinolin-2(1H)-one obtained in Reference Example 75, the title compound was obtained as crystals. yield 20%.

melting point 194-195° C. (ethyl acetate-hexane)

¹H-NMR (CDCl₃) δ: 2.75-2.83 (2H, s), 3.00-3.08 (2H, s), 4.69 (2H, d, J=6.0 Hz), 6.46 (1H, d, J=8.1 Hz), 6.78 (1H, dt, J=5.4, 1.5 Hz), 6.92 (1H, s), 7.00-7.30 (4H, m), 7.36 (1H, d, J=7.8 Hz), 7.62 (1H, dt, J=7.8, 1.5 Hz), 8.28 (1H, d, J=5.4 Hz), 8.55 (1H, d, J=4.8 Hz), 9.07 (1H, s), 9.78 (1H, s).

### Example 68

### 1-(4-(3-oxo-2,3-dihydro-4H-1,4-benzoxazin-4-yl)pyridin-2-yl)-3-(pyridin-2-ylmethyl)urea

In the same manner as in Example 61 and using 4-(2-aminopyridin-4-yl)-2H-1,4-benzoxazin-3(4H)-one obtained in Reference Example 76, the title compound was obtained as crystals. yield 21%.

melting point 206-207° C. (ethyl acetate-hexane)

¹H-NMR (CDCl₃) δ: 4.70 (2H, d, J=5.4 Hz), 4.73 (2H, s), 6.56 (1H, d, J=7.5 Hz), 6.81-7.00 (3H, m), 7.01-7.20 (3H, m), 7.35 (1H, d, J=7.5 Hz), 7.63 (1H, dt, J=7.8, 1.5 Hz), 8.33 (1H, d, J=5.4 Hz), 8.56 (1H, d, J=4.5 Hz), 8.99 (1H, s), 9.77 (1H, br s).

### Example 69

### N-(2-(((pyridin-2-ylmethyl)carbamoyl)amino)pyridin-4-yl)benzamide

In the same manner as in Example 42 and using N-(2-aminopyridin-4-yl)benzamide obtained in Reference Example 77, the title compound was obtained as crystals. yield 61%.

melting point 222-223° C. (ethanol-hexane)

¹H-NMR (DMSO-d₆) δ: 4.49 (2H, d, J,=5.7 Hz), 7.23-7.42 (3H, m), 7.50-7.65 (3H, m), 7.77 (1H, t, J=7.5 Hz), 7.86 (1H, s), 7.95 (2H, dd, J=7.2, 0.6 Hz), 8.09 (1H, d, J=5.7 Hz), 8.52 (1H, d, J=4.8 Hz), 8.82-8.95 (1H, m), 9.39 (1H, s), 10.6 (1H, s).

### Example 70

### N-(2-(((pyridin-2-ylmethyl)carbamoyl)amino)pyridin-4-yl)pyridine-2-carboxamide

In the same manner as in Example 42 and using N-(2-aminopyridin-4-yl)pyridine-2-carboxamide obtained in Reference Example 78, the title compound was obtained as crystals. yield 48%.

melting point 206-207° C. (ethyl acetate-hexane)

¹H-NMR (CDCl₃) δ: 4.74 (2H, d, J=5.7 Hz), 7.14-7.21 (2H, m), 7.37-7.43 (2H, m), 7.49-7.56 (1H, m), 7.65 (2H, dt, J=7.8, 4.8 Hz), 7.94 (1H, dt, J=7.8, 4.8 Hz), 8.17 (1H, d, J=5.7 Hz), 8.28 (1H, dd, J=8.1, 1.2 Hz), 8.54-8.64 (2H, m), 9.95 (1H, br s), 10.2 (1H, s).

### Example 71

### 2-phenyl-N-(2-(((pyridin-2-ylmethyl)carbamoyl)amino)pyridin-4-yl)acetamide

In the same manner as in Example 42 and using N-(2-aminopyridin-4-yl)-2-phenylacetamide obtained in Reference Example 79, the title compound was obtained as crystals. yield 42%.

melting point 219-220° C. (ethyl acetate)

¹H-NMR (DMSO-d₆) δ: 3.66 (2H, s), 4.47 (2H, d, J=5.4 Hz), 7.21-7.33 (8H, m), 7.57 (1H, d, J=1.2 Hz), 7.75 (1H, dt, J=7.5, 2.1 Hz), 8.03 (1H, d, J=5.7 Hz), 8.51 (1H, d, J=4.5 Hz), 8.80 (1H, br s), 9.34 (1H, s), 10.5 (1H, br s).

### Example 72

### bis(2,2,2-trichloroethyl)(4-((tert-butoxycarbonyl)amino)pyridin-2-yl)imidodicarbonate

In the same manner as in Example 20 and using tert-butyl carbamate, the title compound was obtained. yield 50%.

¹H-NMR (CDCl₃) δ: 1.53 (9H, s), 4.80 (4H, s), 7.28 (1H, s), 7.53 (1H, s), 8.34 (1H, d, J=5.5 Hz).

### Example 73

### tert-butyl (2-((((pyridin-2-ylmethyl)amino)carbonyl)amino)pyridin-4-yl)carbamate

In the same manner as in Example 21 and using bis(2,2,2-trichloroethyl)(4-((tert-butoxycarbonyl)amino)pyridin-2-yl)imidodicarbonate obtained in Example 72, the title compound was obtained. yield 55%.

melting point 192-193° C. (ethanol)

¹H-NMR (DMSO-d₆) δ: 1.45 (9H, s), 4.46 (2H, d, J=5.8 Hz), 6.92 (1H, dd, J=5.8, 1.7 Hz), 7.24 (1H, dd, J=7.7, 4.9 Hz), 7.94 (1H, d, J=5.8 Hz), 8.50 (1H, d, J=4.1 Hz), 8.96 (1H, brs), 9.25 (1H, s), 9.77 (1H, s).

The compounds obtained in Examples 1-73 are shown in the following Table 1.

TABLE 1

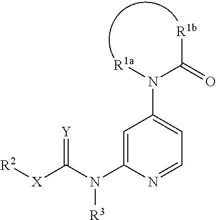

| Ex. No. | 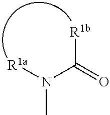 | R² | R³ | X | Y |
|---|---|---|---|---|---|
| 1 | 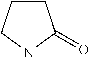 | Bn | H | NH | O |
| 2 | 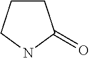 | Et | H | NH | O |
| 3 | 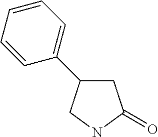 | Bn | H | NH | O |
| 4 | 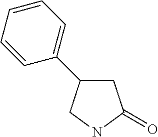 | 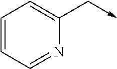 | H | NH | O |
| 5 | 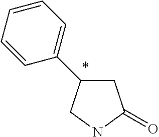 | 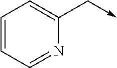 | H | NH | O |
| 6 | 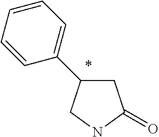 | 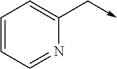 | H | NH | O |
| 7 | 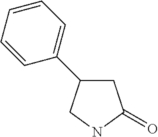 | 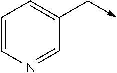 | H | NH | O |
| 8 | 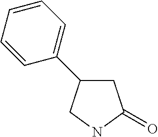 | 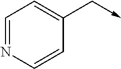 | H | NH | O |
| 9 | 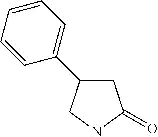 | 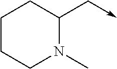 | H | NH | O |
| 10 | 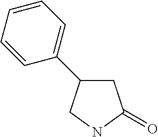 | 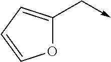 | H | NH | O |
| 11 | 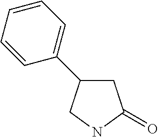 | 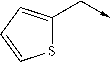 | H | NH | O |
| 12 | 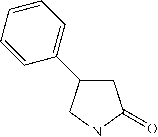 | 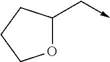 | H | NH | O |
| 13 | 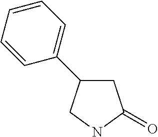 | 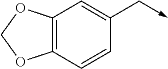 | H | NH | O |
| 14 | 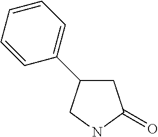 | 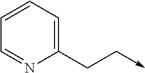 | H | NH | O |
| 15 | 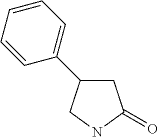 | 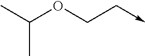 | H | NH | O |
| 16 | 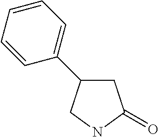 | 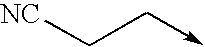 | H | NH | O |
| 17 | 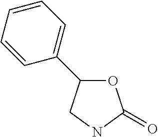 | 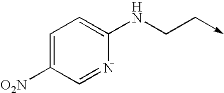 | H | NH | O |
| 18 | 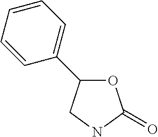 | 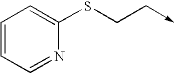 | H | NH | O |
| 19 | 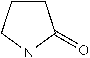 | 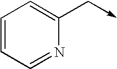 | H | NH | O |
| 20 | 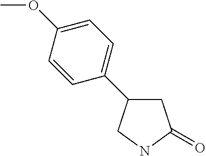 | 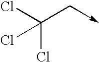 | Troc | O | O |
| 21 | 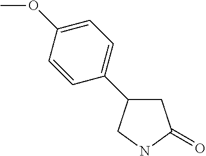 | 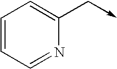 | H | NH | O |
| 22 | 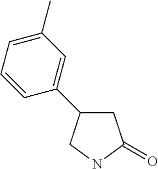 | 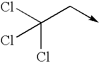 | Troc | O | O |
| 23 | 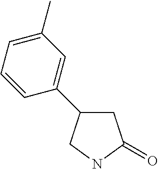 | 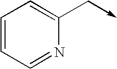 | H | NH | O |
| 24 | 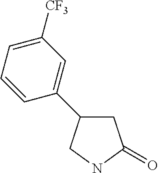 | 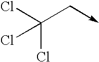 | Troc | O | O |
| 25 | 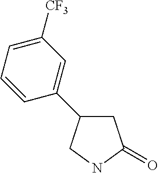 | 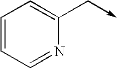 | H | NH | O |
| 26 | 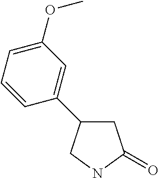 | 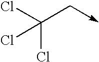 | Troc | O | O |
| 27 | 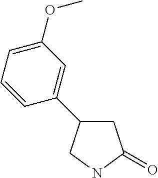 | 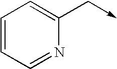 | H | NH | O |
| 28 | 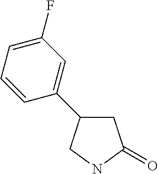 | 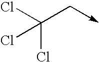 | Troc | O | O |
| 29 | 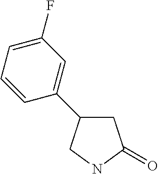 | 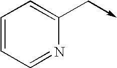 | H | NH | O |
| 30 | 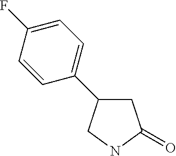 | 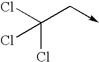 | Troc | O | O |
| 31 | 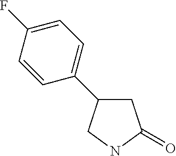 | 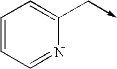 | H | NH | O |
| 32 | 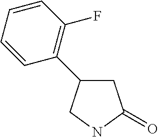 | 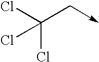 | Troc | O | O |
| 33 | 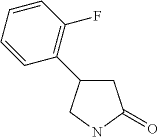 | 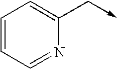 | H | NH | O |
| 34 | 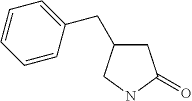 | 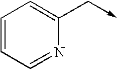 | Troc | O | O |
| 35 | 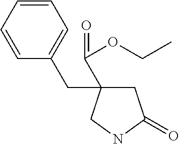 | 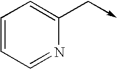 | H | NH | O |
| 36 | 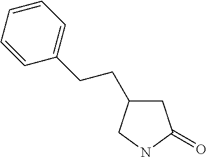 | 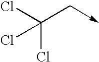 | Troc | O | O |
| 37 | 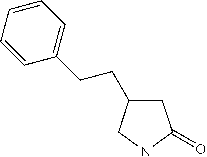 | 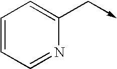 | H | NH | O |
| 38 | 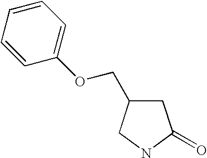 | 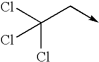 | Troc | O | O |
| 39 | 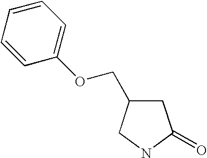 | 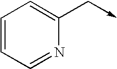 | H | NH | O |
| 40 | 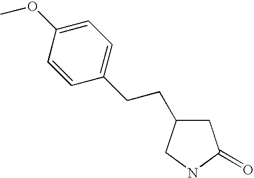 | 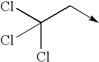 | Troc | O | O |
| 41 | 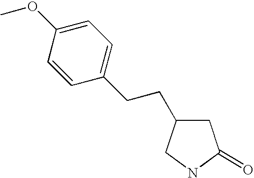 | 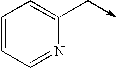 | H | NH | O |
| 42 | 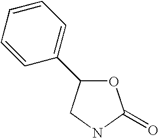 | 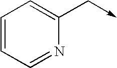 | H | NH | O |
| 43 | 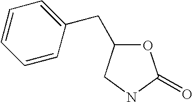 | 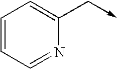 | H | NH | O |
| 44 | 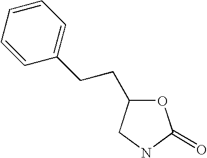 | 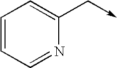 | H | NH | O |
| 45 | 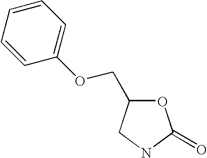 | 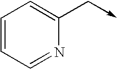 | H | NH | O |
| 46 | 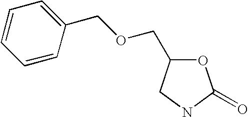 | 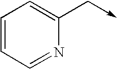 | H | NH | O |
| 47 | 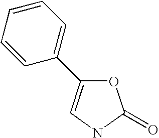 | 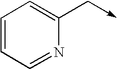 | H | NH | O |
| 48 | 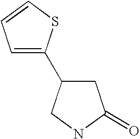 | 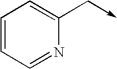 | H | NH | O |
| 49 | 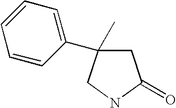 | 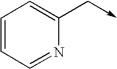 | H | NH | O |
| 50 | 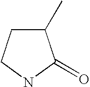 | 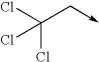 | Troc | O | O |
| 51 | 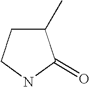 | 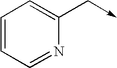 | H | NH | O |
| 52 | 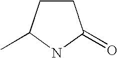 | 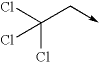 | Troc | O | O |
| 53 | 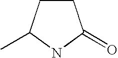 | 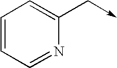 | H | NH | O |
| 54 | 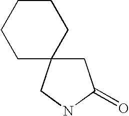 | 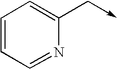 | H | NH | O |
| 55 | 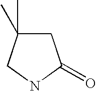 | 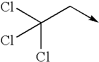 | Troc | O | O |
| 56 | 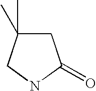 | 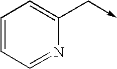 | H | NH | O |
| 57 | 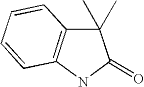 | Bn | H | NH | O |
| 58 | 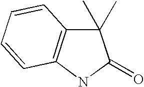 | 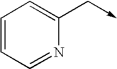 | H | NH | O |
| 59 | 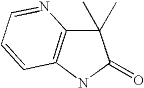 | 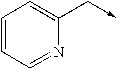 | H | NH | O |
| 60 | 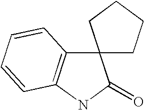 | 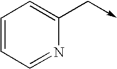 | H | NH | O |
| 61 | 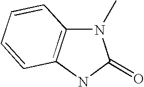 | 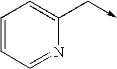 | H | NH | O |
| 62 | 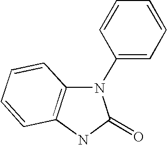 | 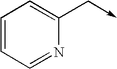 | H | NH | O |
| 63 | 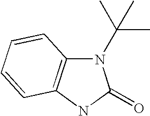 | 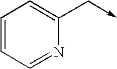 | H | NH | O |
| 64 | 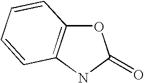 | 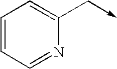 | H | NH | O |
| 65 | 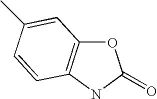 | 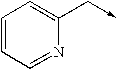 | H | NH | O |
| 66 | 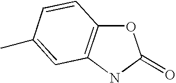 | 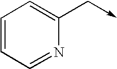 | H | NH | O |
| 67 | 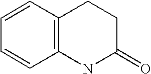 | 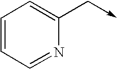 | H | NH | O |
| 68 | 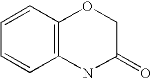 | 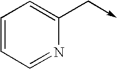 | H | NH | O |
| 69 | 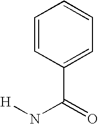 | 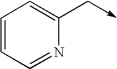 | H | NH | O |
| 70 | 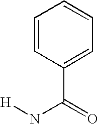 | 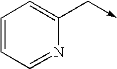 | H | NH | O |
| 71 | 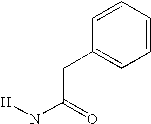 | 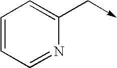 | H | NH | O |
| 72 | 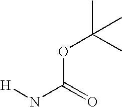 | 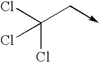 | Troc | O | O |
| 73 | 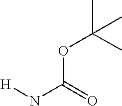 | 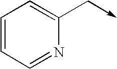 | H | NH | O |

### Example 74

### N-(4-(2-oxopyrrolidin-1-yl)pyridin-2-yl)-2-(thiophen-2-yl)acetamide

To a solution (1 ml) of 1-(2-aminopyridin-4-yl)pyrrolidin-2-one (106 mg, 0.6 mmol) obtained in Reference Example 3 and triethylamine (52 μl, 0.72 mmol) in tetrahydrofuran was added 2-(thiophen-2-yl)acetyl chloride (89 μl, 0.72 mmol) at 0° C., and the reaction mixture was stirred at 80° C. for 24 hr. The mixture was diluted with water, and extracted with ethyl acetate. The extract was washed with water, and dried over anhydrous magnesium sulfate. The solvent was evaporated under reduced pressure, and the obtained residue was purified by silica gel column chromatography (ethyl acetate) to give the title compound (100 mg, yield 55%).

melting point 197-198° C. (ethanol)

¹H-NMR (DMSO-d₆) δ: 2.06 (2H, m), 2.39-2.60 (2H, m), 3.80 (2H, t, J=7.1 Hz), 3.95 (2H, s), 6.92-6.99 (2H, m), 7.38 (1H, dd, J=4.1, 1.9 Hz), 7.53 (1H, dd, J=5.8, 1.9 Hz), 8.21 (1H, d, J=5.8 Hz), 8.30 (1H, s), 10.68 (1H, s).

### Example 75

### N-(4-(2-oxo-4-phenylpyrrolidin-1-yl)pyridin-2-yl)-3-(pyridin-2-yl)propanamide

A solution (5 ml) of 1-(2-aminopyridin-4-yl)-4-phenylpyrrolidin-2-one (253 mg, 1 mmol) obtained in Reference Example 4, WSC (230 mg, 1.2 mmol), HOBt (162 mg, 1.2 mmol) and 3-(pyridin-2-yl)propanoic acid (181 mg, 1.2 mmol) in 1-methylpyrrolidin-2-one was stirred with irradiating microwave at 100° C. for 20 min. The mixture was diluted with water, and extracted with diethyl ether. The extract was washed with water, and dried over anhydrous magnesium sulfate. The solvent was evaporated under reduced pressure, and the obtained residue was purified by silica gel column chromatography (ethyl acetate) to give the title compound (30 mg, yield 8%) as crystals. melting point 145-146° C. (ethanol)

¹H-NMR (DMSO-d₆) δ: 2.72-2.92 (4H, m), 3.02 (2H, t, J=7.3 Hz), 3.61-3.85 (2H, m), 4.18 (1H, t, J=7.7 Hz), 7.10-7.21 (1H, m), 7.21-7.44 (6H, m), 7.50 (1H, d, J=4.9 Hz), 7.67 (1H, t, J=7.4 Hz), 8.19 (1H, d, J=5.5 Hz), 8.34 (1H s), 8.45 (1H, d, J=4.4 Hz), 10.46 (1H, brs).

### Example 76

### N-(4-(2-oxo-5-phenyl-1,3-oxazolidin-3-yl)pyridin-2-yl)-3-(pyridin-2-yl)propanamide

In the same manner as in Example 75, and using 3-(2-aminopyridin-4-yl)-5-phenyl-1,3-oxazolidin-2-one obtained in Reference Example 42 and 3-(pyridin-2-yl)propanoic acid, the title compound was obtained. yield 30%.

melting point 169-171° C. (ethanol)

¹H-NMR (DMSO-d₆) δ: 2.84 (2H, t, J=7.3 Hz), 3.03 (2H, t, J=7.3 Hz), 3.96 (1H, dd, J=8.9, 8.1 Hz), 4.49 (1H, t, J=8.9 Hz), 5.78 (1H, t, J=8.1 Hz), 7.18 (1H, dd, J=7.6, 4.7 Hz), 7.28 (1H, d, J=7.6 Hz), 7.34 (1H, dd, J=5.8, 1.9 Hz), 7.39-7.56 (5H, m), 7.68 (1H, td, J=7.6, 1.9 Hz), 8.21 (1H, d, J=5.8 Hz), 8.33 (1H, d, J=1.9 Hz), 8.46 (1H, d, J=4.7 Hz), 10.51 (1H, s)

### Example 77

### N-(4-(2-oxo-5-phenyl-1,3-oxazolidin-3-yl)pyridin-2-yl)-3-(pyridin-3-yl)propanamide

In the same manner as in Example 75, and using 3-(2-aminopyridin-4-yl)-5-phenyl-1,3-oxazolidin-2-one obtained in Reference Example 42 and 3-(pyridin-3-yl)propanoic acid, the title compound was obtained as crystals. yield 73%.

melting point 171-172° C. (ethanol)

¹H-NMR (DMSO-d₆) δ: 2.73 (2H, t, J=7.4 Hz), 2.90 (2H, t, J=7.4 Hz), 3.95 (1H, dd, J=8.9, 8.1 Hz), 4.48 (1H, t, J=8.9 Hz), 5.78 (1H, t, J=8.1 Hz), 7.25-7.34 (2H, m), 7.39-7.48 (3H, m), 7.48-7.54 (2H, m), 7.64 (1H, dt, J=7.9, 1.9 Hz), 8.20 (1H, d, J=5.8 Hz), 8.33 (1H, d, J=1.9 Hz), 8.37 (1H, dd, J=4.7, 1.6 Hz), 8.45 (1H, d, J=1.9 Hz), 10.48 (1H, s).

### Example 78

### 3-(1,3-benzothiazol-2-yl)-N-(4-(2-oxo-5-phenyl-1,3-oxazolidin-3-yl)pyridin-2-yl)propanamide

In the same manner as in Example 75 and using 3-(benzothiazol-2-yl)propanoic acid, the title compound was obtained. yield 18%.

melting point 229-230° C. (ethanol)

¹H-NMR (DMSO-d₆) δ: 3.30 (2H, t, J=7.0 Hz), 3.41 (2H, t, J=7.0 Hz), 3.96 (1H, t, J=8.5 Hz), 4.49 (1H, t, J=9.1 Hz), 5.78 (1H, t, J=8.1 Hz), 7.27-7.57 (8H, m), 7.91 (1H, d, J=8.3 Hz), 8.03 (1H, d, J=7.6 Hz), 8.23 (1H,d, J=5.7 Hz), 8.34 (1H, s), 10.63 (1H, s).

### Example 79

### N-(4-(2-oxo-5-phenyl-1,3-oxazolidin-3-yl)pyridin-2-yl)-2-(2-thienyl)acetamide

In the same manner as in Example 75 and using 2-(thiophen-2-yl)acetic acid, the title compound was obtained. yield 17%.

melting point 109-110° C. (ethyl acetate-hexane)

¹H-NMR (DMSO-d₆) δ: 3.87-4.08 (3H, m), 4.49 (1H, t, J=8.9 Hz), 5.78 (1H, t, J=8.1 Hz), 6.90-7.02 (2H, m), 7.31-7.58 (7H, m), 8.24 (1H, d, J=5.8 Hz), 8.32 (1H, d, J=1.9 Hz), 10.72 (1H, s).

### Example 80

### N-(4-(2-oxo-phenylpyrrolidin-1-yl)pyridin-2-yl)benzamide

A mixture of 1-(2-aminopyridin-4-yl)-4-phenylpyrrolidin-2-one (15.2 mg, 0.06 mmol), benzoyl chloride (0.09 mmol), triethylamine (0.12 mmol) and N,N-dimethylacetamide (0.5 ml) was stirred at 80° C. for 24 hr. The reaction mixture was purified by preparative HPLC to give the title compound (5.8 mg, yield 27%) as an oil.

LC-MS 358 (M+H)

¹H-NMR (CDCl₃) δ: 2.83-2.93 (1H, m), 3.02-3.11 (1H, m), 3.69-3.80 (1H, m), 3.97 (1H, J=7.8 Hz and J=9.8 Hz), 4.33 (1H, dd, J=9.7, 8.2 Hz), 7.28-7.34 (3H, m) 7.36-7.42 (2H, m), 7.51 (2H, t, J=7.3 Hz), 7.56-7.61 (1H, m), 7.89-7.94 (2H, m), 8.09 (1H, dd, J=5.9, 2.2 Hz), 8.21-8.26 (2H, m), 8.73 (1H, s).

The compounds obtained in Examples 74-80 are shown in the following Table 2.

TABLE 2

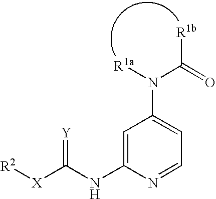

| Ex. No. | 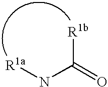 | 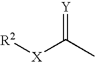 |
|---|---|---|
| 74 | 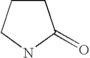 | 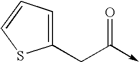 |
| 75 | 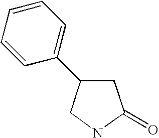 | 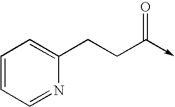 |
| 76 | 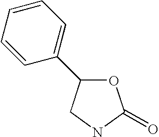 | 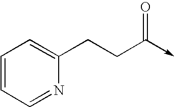 |
| 77 | 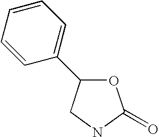 | 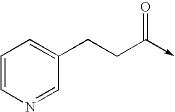 |
| 78 | 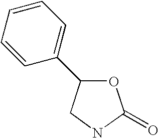 | 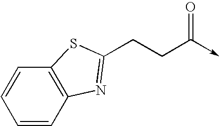 |
| 79 | 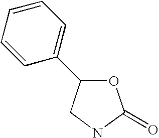 | 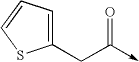 |
| 80 | 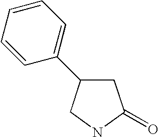 | 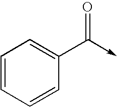 |

### Examples 81-119

In the same manner as in Example 80, the reaction of 1-(2-aminopyridin-4-yl)-4-phenylpyrrolidin-2-one with various acid chlorides or isocyanates was performed under similar conditions except that triethylamine was not added when the reagent was isocyanate to give the compounds of Examples 81-119. The synthesized compounds are shown in Table 3.

TABLE 3

| Example No. | Chemical Structure | MS (m/z) |
|---|---|---|
| 81 | 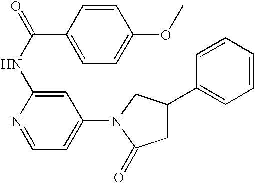 | 388 |
| 82 | 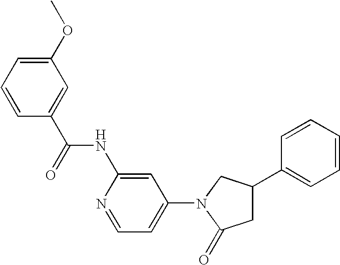 | 388 |
| 83 | 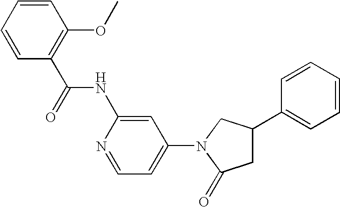 | 388 |
| 84 | 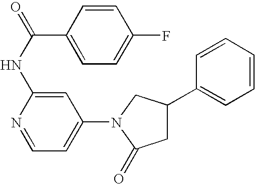 | 376 |
| 85 | 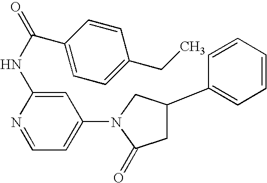 | 386 |
| 86 | 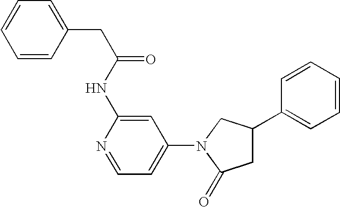 | 372 |
| 87 | 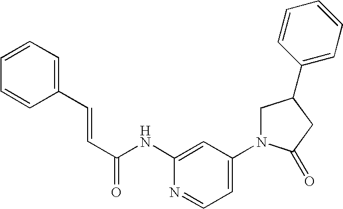 | 384 |
| 88 | 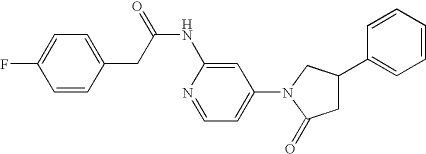 | 390 |
| 89 | 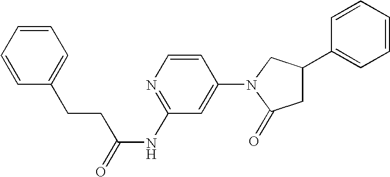 | 386 |
| 90 | 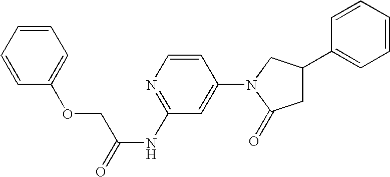 | 388 |
| 91 | 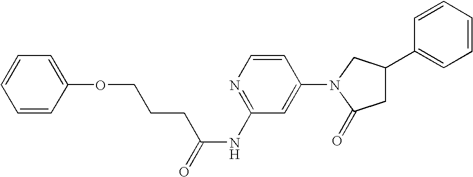 | 416 |
| 92 | 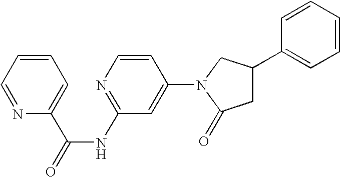 | 359 |
| 93 | 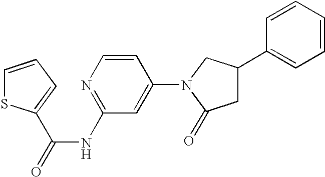 | 364 |
| 94 | 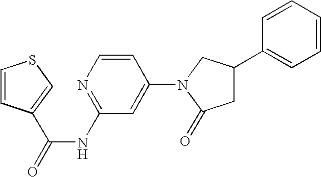 | 364 |
| 95 | 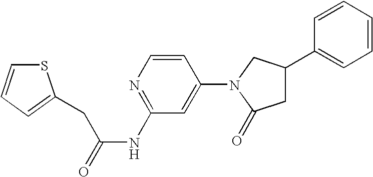 | 378 |
| 96 | 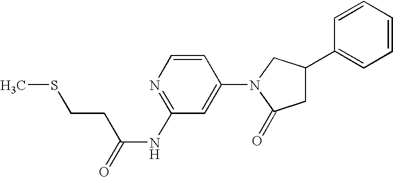 | 356 |
| 97 | 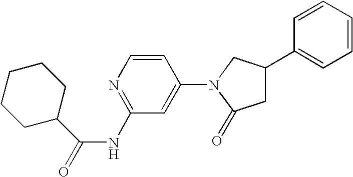 | 364 |
| 98 | 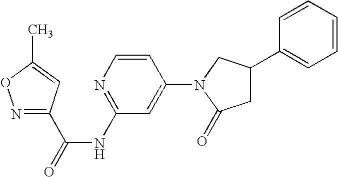 | 363 |
| 99 | 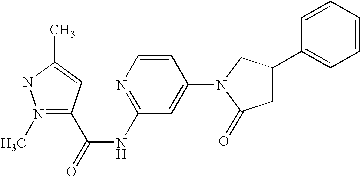 | 376 |
| 100 | 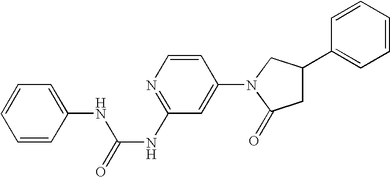 | 373 |
| 101 | 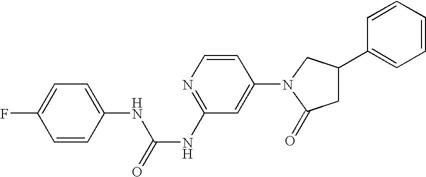 | 391 |
| 102 | 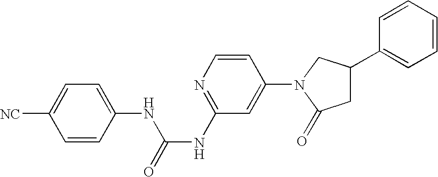 | 398 |
| 103 | 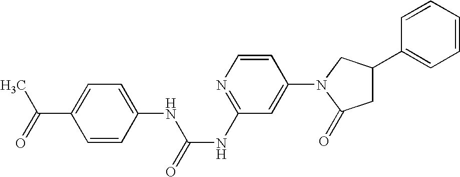 | 415 |
| 104 | 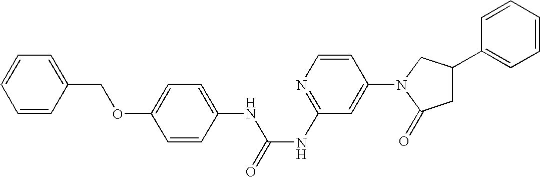 | 479 |
| 105 | 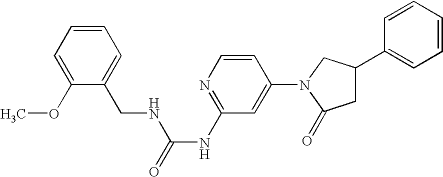 | 417 |
| 106 | 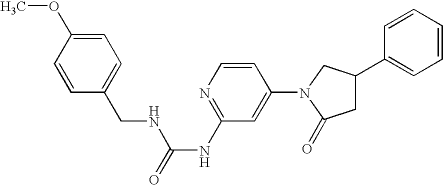 | 417 |
| 107 | 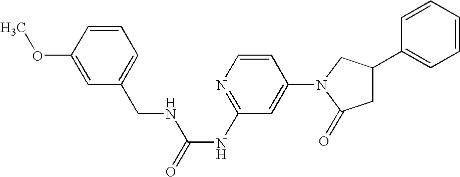 | 417 |
| 108 | 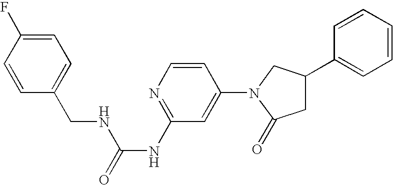 | 405 |
| 109 | 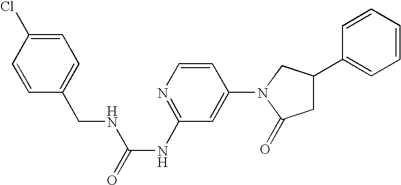 | 421 |
| 110 | 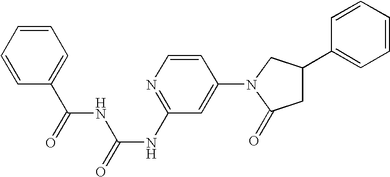 | 401 |
| 111 | 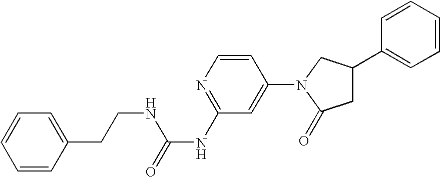 | 401 |
| 112 | 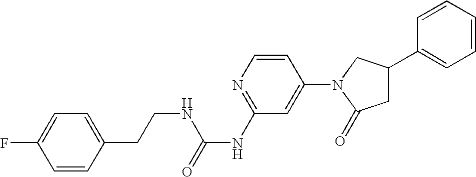 | 419 |
| 113 | 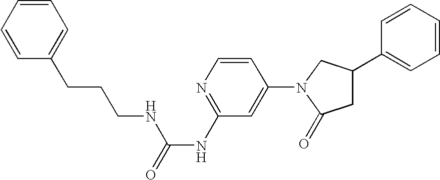 | 415 |
| 114 | 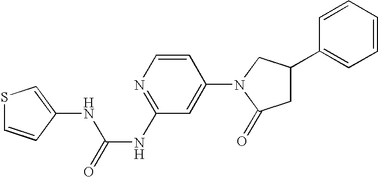 | 379 |
| 115 | 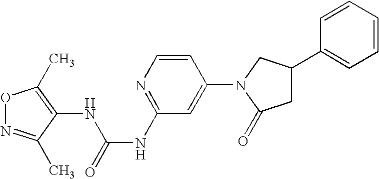 | 392 |
| 116 | 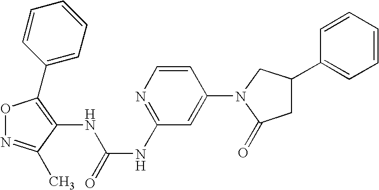 | 454 |
| 117 | 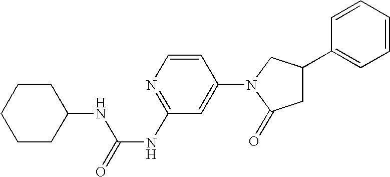 | 379 |
| 118 | 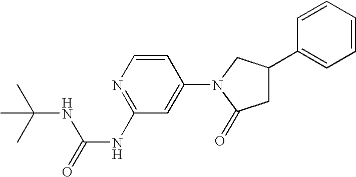 | 353 |
| 119 | 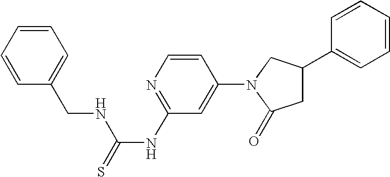 | 402 |

### Example 120

### N-benzyl-N′-(5-(2-oxopyrrolidin-1-yl)pyridin-2-yl)urea

In the same manner as in Example 20, step 1 and using N-benzyl-N′-(5-bromopyridin-2-yl)urea obtained in Reference Example 80 and pyrrolidin-2-one, the title compound was obtained. yield 60%.

melting point 185-186° C. (tetrahydrofuran)

¹H-NMR (DMSO-d₆) δ: 1.99-2.09 (2H, m), 2.42-2.49 (2H, m), 3.78 (2H, t, J=7.0 Hz), 4.37 (2H, d, J=6.0 Hz), 7.20-7.32 (5H, m), 7.43 (1H, d, J=9.1 Hz), 7.99 (1H, dd, J=9.1, 2.7 Hz), 8.29 (1H, br s), 8.42 (1H, d, J=2.7 Hz), 9.25 (1H, br s).

### Example 121

### N-benzyl-N′-(5-(2-oxo-4-phenylpyrrolidin-1-yl)pyridin-2-yl)urea

In the same manner as in Example 20, step 1 and using N-benzyl-N′-(5-bromopyridin-2-yl)urea obtained in Reference Example 80 and 4-phenylpyrrolidin-2-one, the title compound was obtained. yield 56%.

melting point 172-173° C. (tetrahydrofuran)

¹H-NMR (DMSO-d₆) δ: 2.64-2.77 (1H, m), 2.81-2.93 (1H, m), 3.74 (1H, m), 3.84 (1H, t, J=8.5 Hz), 4.17 (1H, t, J=8.5 Hz), 4.39 (2H, d, J=5.8 Hz), 7.19-7.43 (10H, m), 7.47 (1H, d, J=9.1 Hz), 8.03 (1H, dd, J=9.1, 2.7 Hz), 8.29 (1H, br s), 8.49 (1H, d, J=2.5 Hz).

### Examples 122-174

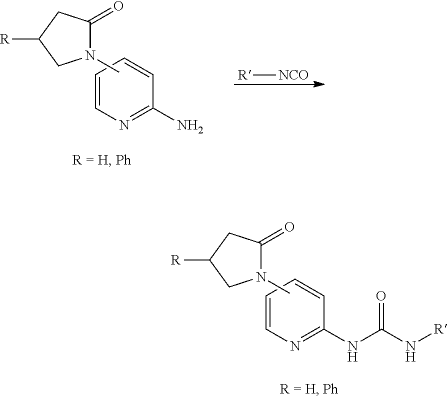

Library synthesis was performed using a glass tube (13 mL), micro stirring rack and Orbital Shaker on a 40 micro mol-scale. To 1-(2-aminopyridin-4-yl)pyrrolidin-2-one obtained in Reference Example 3, 1-(2-aminopyridin-4-yl)-4-phenylpyrrolidin-2-one obtained in Reference Example 4, 1-(6-aminopyridin-3-yl)pyrrolidin-2-one obtained in Reference Example 81 or 1-(2-aminopyridin-3-yl)pyrrolidin-2-one obtained in Reference Example 82 (40 μmol, THF/DMA=1/1, 1.0 mL solution) was added isocyanic acid R′NCO (60 μmol, 1.5 eq), and the mixture was stirred at room temperature for 1 hr. Ethyl acetate (3 mL) and 5% aqueous sodium hydrogen carbonate solution (1 mL) were added to the mixture, and the mixture was vigorously stirred using Orbital Shaker. The organic layer was separated using Tecan dispenser and upper layer Phase Sep, and the solvent was evaporated using soltrapper. The residue was dissolved in DMSO/MeOH=1/1 (0.5 mL), and purified by reversed-phase preparative apparatus to give the object compound shown in Table 4.

### N-(3,5-dimethoxyphenyl)-N′-[3-(2-oxopyrrolidin-1-yl)pyridin-2-yl]urea (Example 142)

¹H-NMR (400 MHz, MeOD) δ: 2.25 (2H, dt, J=14.98, 7.91, 7.63 Hz) 2.65 (2H, t, J=8.10 Hz) 3.78 (6H, s) 3.94 (2H, t, J=7.06 Hz) 6.28 (1H, t, J=1.98 Hz) 6.78 (1H, d, J=2.26 Hz) 7.46 (1H, d, J=9.42 Hz) 8.55 (1H, dd, J=9.61, 2.45 Hz) 8.85 (1H, d, J=2.45 Hz)

### N-[4-(2-oxo-4-phenylpyrrolidin-1-yl)pyridin-2-yl]-N′-(2-phenylethyl)urea (Example 160)

¹H-NMR (400 MHz, MeOD) δ: 2.87 (2H, t, J=7.16 Hz) 2.95 (1H, d, J=9.98 Hz) 3.03 (1H, d, J=8.48 Hz) 3.52 (2H, t, J=7.16 Hz) 3.74-3.86 (1H, m) 3.91-3.98 (1H, m) 4.35 (1H, dd, J=9.32, 8.57 Hz) 7.17-7.41 (10H, m) 7.60 (1H, dd, J=7.16, 2.07 Hz) 7.73 (1H, d, J=0.56 Hz) 8.11 (1H, d, J=7.16 Hz)

TABLE 4

| Example No. | Chemical Structure | salt | MS(m/Z) |
|---|---|---|---|
| 122 | 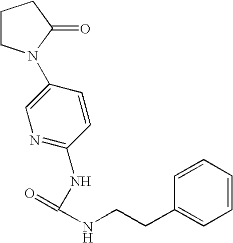 | CF3COOH | 438.40 |
| 123 | 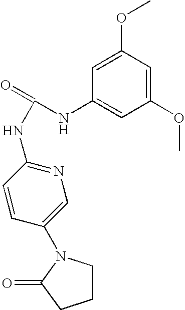 | CF3COOH | 470.40 |
| 124 | 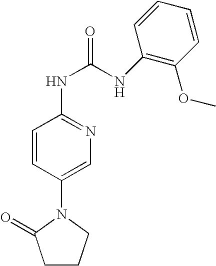 | CF3COOH | 440.37 |
| 125 | 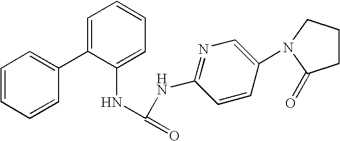 | CF3COOH | 486.44 |
| 126 | 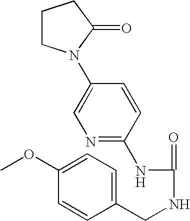 | CF3COOH | 454.40 |
| 127 | 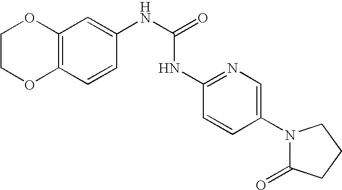 | CF3COOH | 468.38 |
| 128 | 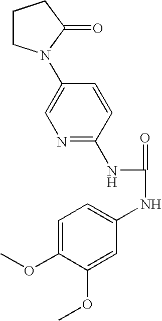 | CF3COOH | 470.40 |
| 129 | 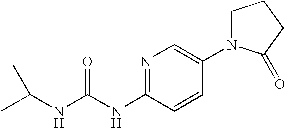 | CF3COOH | 376.33 |
| 130 | 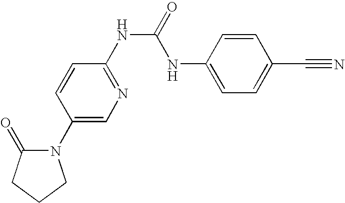 | CF3COOH | 435.36 |
| 131 | 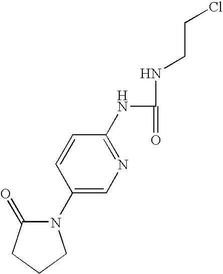 | CF3COOH | 396.75 |
| 132 | 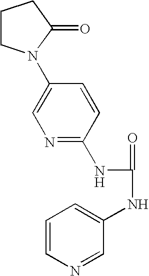 | 2CF3COOH | 525.36 |
| 133 | 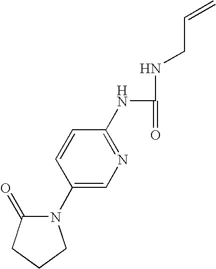 | CF3COOH | 374.32 |
| 134 | 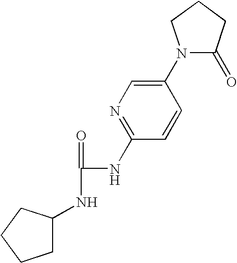 | CF3COOH | 402.37 |
| 135 | 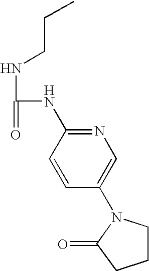 | CF3COOH | 376.33 |
| 136 | 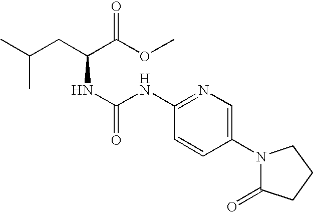 | CF3COOH | 462.42 |
| 137 | 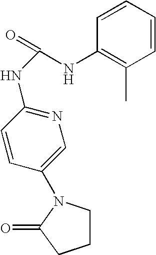 | CF3COOH | 424.37 |
| 138 | 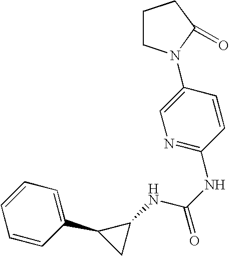 | CF3COOH | 450.41 |
| 139 | 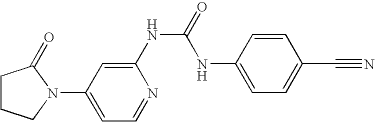 | CF3COOH | 435.36 |
| 140 | 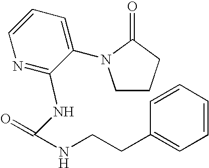 | CF3COOH | 438.40 |
| 141 | 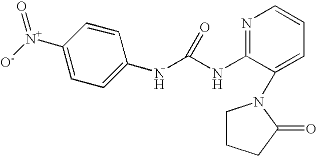 | CF3COOH | 455.34 |
| 142 | 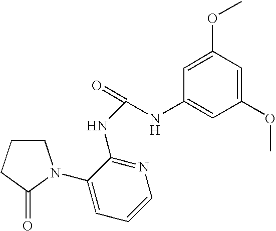 | CF3COOH | 470.40 |
| 143 | 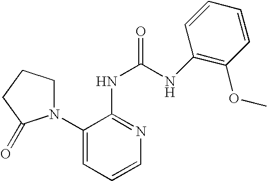 | CF3COOH | 440.37 |
| 144 | 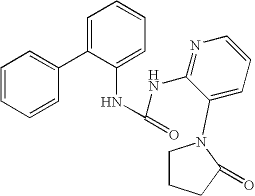 | CF3COOH | 486.44 |
| 145 | 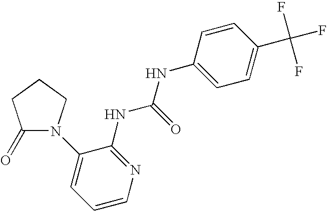 | CF3COOH | 478.35 |
| 146 | 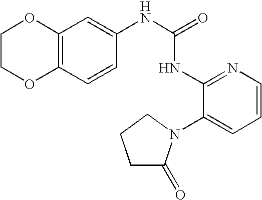 | CF3COOH | 468.38 |
| 147 | 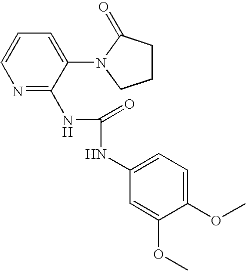 | CF3COOH | 470.40 |
| 148 | 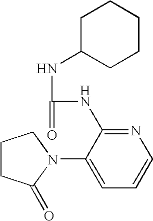 | CF3COOH | 416.39 |
| 149 | 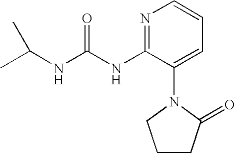 | CF3COOH | 376.33 |
| 150 | 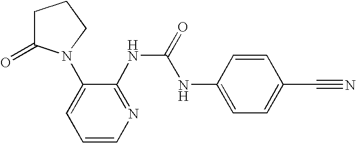 | CF3COOH | 435.36 |
| 151 | 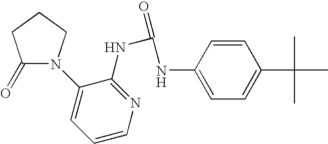 | CF3COOH | 466.45 |
| 152 | 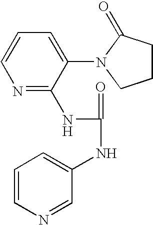 | 2CF3COOH | 525.36 |
| 153 | 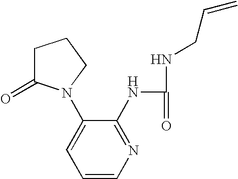 | CF3COOH | 374.32 |
| 154 | 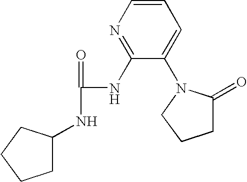 | CF3COOH | 402.37 |
| 155 | 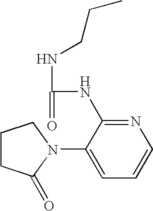 | CF3COOH | 376.33 |
| 156 | 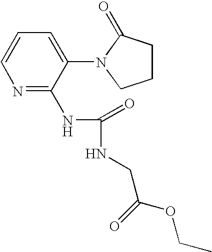 | CF3COOH | 420.34 |
| 157 | 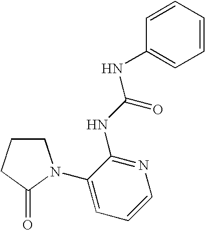 | CF3COOH | 410.35 |
| 158 | 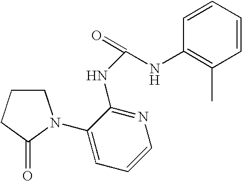 | CF3COOH | 424.37 |
| 159 | 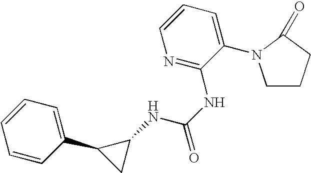 | CF3COOH | 450.41 |
| 160 | 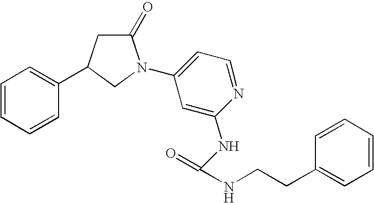 | CF3COOH | 514.50 |
| 161 | 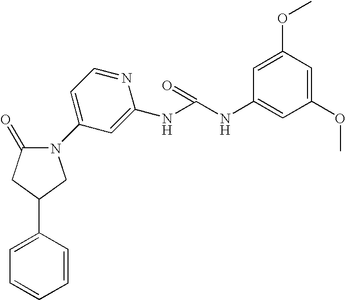 | CF3COOH | 546.50 |
| 162 | 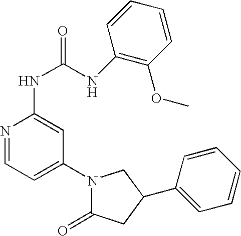 | CF3COOH | 516.47 |
| 163 | 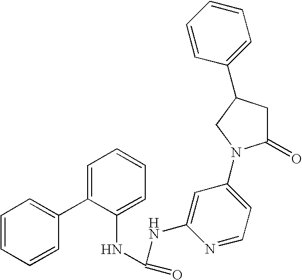 | CF3COOH | 562.54 |
| 164 | 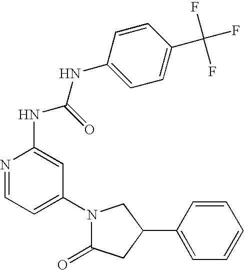 | CF3COOH | 554.44 |
| 165 | 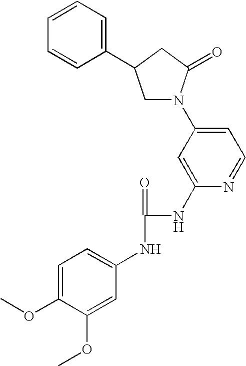 | CF3COOH | 546.50 |
| 166 | 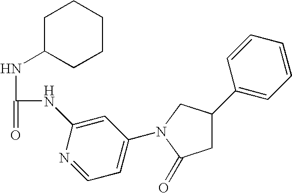 | CF3COOH | 492.49 |
| 167 | 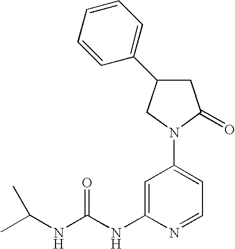 | CF3COOH | 452.43 |
| 168 | 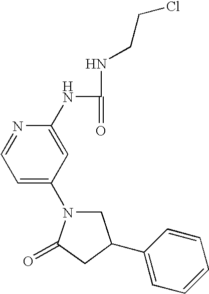 | CF3COOH | 472.85 |
| 169 | 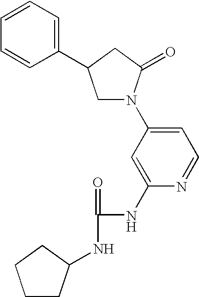 | CF3COOH | 478.46 |
| 170 | 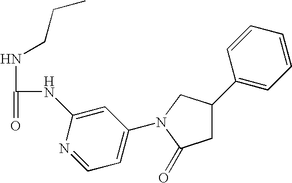 | CF3COOH | 452.43 |
| 171 | 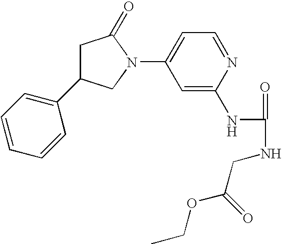 | CF3COOH | 496.44 |
| 172 | 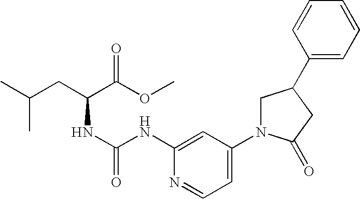 | CF3COOH | 538.52 |
| 173 | 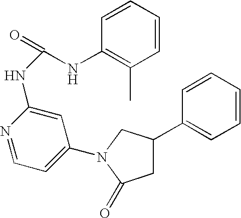 | CF3COOH | 500.47 |
| 174 | 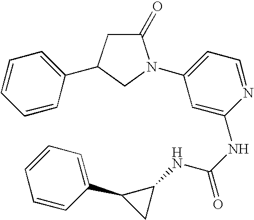 | CF3COOH | 526.51 |

| | |
|---|---|
| (1) compound of Example 1 | 50.0 mg |
| (2) lactose | 34.0 mg |
| (3) cornstarch | 10.6 mg |
| (4) cornstarch (paste) | 5.0 mg |
| (5) magnesium stearate | 0.4 mg |
| (6) calcium carboxymethylcellulose | 20.0 mg |
| total | 120.0 mg |

According to a conventional method, the above-mentioned (1)-(6) are mixed and tableted by a tableting machine to give a tablet.

### Formulation Example 2

| | |
|---|---|
| (1) the compound of Example 1 | 10.0 mg |
| (2) lactose | 60.0 mg |
| (3) cornstarch | 35.0 mg |
| (4) gelatin | 3.0 mg |
| (5) magnesium stearate | 2.0 mg |

A mixture of the compound (10.0 mg) of Example 1, lactose (60.0 mg) and cornstarch (35.0 mg) is granulated using 10% aqueous gelatin solution (0.03 mL, 3.0 mg as gelatin) and by passing through a 1 mm mesh sieve, dried at 40° C. and passed through a sieve again. The thus-obtained granules are mixed with magnesium stearate (2.0 mg), and the mixture is compressed. The obtained core tablet is coated with a sugar coating of a suspension of saccharose, titanium dioxide, talc and gum arabic in water. The coated tablet is polished with beeswax to give a coated tablet.

### Formulation Example 3

| | |
|---|---|
| (1) the compound of Example 1 | 10.0 mg |
| (2) lactose | 70.0 mg |
| (3) cornstarch | 50.0 mg |
| (4) soluble starch | 7.0 mg |
| (5) magnesium stearate | 3.0 mg |

The compound of Example 1 (10.0 mg) and magnesium stearate (3.0 mg) are granulated with an aqueous soluble starch solution (0.07 mL, 7.0 mg as soluble starch), dried, and mixed with lactose (70.0 mg) and cornstarch (50.0 mg). The mixture is compressed to give a tablet.

### Experimental Example 1

### GSK-3β Inhibitory Activity Evaluation

### (1) Cloning of Human GSK-3β Gene and Preparation of Recombinant Baculovirus

Human GSK-3β gene was cloned by PCR using human brain cDNA (Clontech; trade name: QUICK-Clone cDNA) as a template and a primer set (GSK3β-U: 5′-AAAGAATTCACCATGGACTACAAGGACGACGATGACAAGTCAGGGCGGCCCAGAACCACCTC CTT-3′ (SEQ ID NO: 1) and GSK3β-L: 5′-AAAAGTCGACTCAGGTGGAGTTGGAAGCTGATGCAGAAG-3′ (SEQ ID NO: 2)) prepared by reference to the base sequence of GSK-3β gene registered under an accession No. NM_{—}002093 in the GenBank.

PCR was performed according to the protocol attached to KOD plus DNA polymerase (TOYOBO CO., LTD.). The obtained PCR product was subjected to agarose gel (1%) electrophoresis, and DNA fragment (1.2 kb) containing GSK-3β gene was recovered from the gel, and digested with restriction enzymes EcoR I and Sal I. DNA after treatment with restriction enzymes was subjected to agarose gel (1%) electrophoresis, and the obtained DNA fragment was recovered, and ligated to plasmid pFASTBAC1 (Invitrogen) digested with restriction enzymes EcoR I and Sal I to give expression plasmid pFB-GSK3β. The base sequence of the inserted fragment was confirmed to match the object sequence. Using BAC-TO-BAC Baculovirus Expression System (Invitrogen), virus stock BAC-GSK3β of recombinant Baculovirus was prepared.

### (2) Preparation of Recombinant GSK-3β Enzyme

Sf-21 cells (Invitrogen) were inoculated to 150 ml Sf-900 II SEM medium (Invitrogen) containing 10% fetal bovine serum to 1×10⁶ cells/ml and cultured at 27° C. for 24 hr. To the obtained culture medium was added the virus stock BAC-GSK3β of recombinant Baculovirus obtained above by 150 μl each, and the cells were further cultured for 60 hr. The culture medium was centrifuged (3000 rpm, 10 min) to separate the cells, and the cells were washed once with PBS. The cells were suspended in 10 ml of cell lysis buffer (25 mM HEPES (pH 7.5), 1% Triton X, 130 mM sodium chloride, 1 mM EDTA, 1 mM Dithiothreitol, 25 mM β-glycerophosphate, Protease inhibitor Complete (Boehringer), 1 mM sodium orthovanadate), treated 4 times in a homogenizer (POLYTRON) at 20000 rpm, 30 sec to disrupt the cells. The cell disrupt solution was centrifuged (40000 rpm, 45 min), and GSK-3β was purified from the obtained supernatant using Anti-FLAG M2 Affinity Gel (Sigma Ltd.).

### (3) Experiment Method

To 37.5 μl of a reaction solution (25 mM HEPES (pH 7.5), 10 mM magnesium acetate, 1 mM DTT, 0.01% bovine serum albumin (Wako Pure Chemical Industries, Ltd.)) containing recombinant GSK-3β enzyme (100 ng) obtained above and substrate peptide (YRRAAVPPSPSLSRHSSPHQpSEDEEE (SEQ ID NO: 3), pS is phosphorylated serine) (100 ng) derived from glycogen synthase was added a test compound (2.5 μl) dissolved in DMSO, and the mixture was incubated at room temperature for 5 min. To the obtained mixture was added ATP solution (2.5 μM ATP, 10 μl), and the mixture was reacted at room temperature for 30 min. After the reaction, the reaction was quenched by adding 50 μL of Kinase Glo Reagent (Promega) to the reaction solution. After reaction at room temperature for 10 min, the luminescence amount was measured using an ARVO multilabel counter (PerkinElmer Life Sciences). The concentration of the test compound necessary for inhibiting the luminescence amount by 50% (IC₅₀ value) was calculated by PRISM 3.0 (Graphpad software). The results are shown in Table 5.

TABLE 5

| Example | IC₅₀ (nM) |
|---|---|
| 4 | <100 |
| 5 | <100 |
| 7 | 190 |
| 13 | 280 |
| 21 | 120 |
| 25 | 100 |
| 29 | 100 |
| 34 | <100 |
| 37 | <100 |
| 42 | <100 |
| 43 | <100 |
| 44 | <100 |
| 45 | <100 |
| 46 | <100 |
| 47 | <100 |
| 48 | <100 |
| 49 | <100 |
| 51 | <100 |
| 53 | 100 |
| 54 | <100 |
| 58 | <100 |
| 59 | <100 |
| 61 | <100 |
| 64 | <100 |
| 65 | <100 |
| 66 | <100 |
| 69 | <100 |
| 75 | 220 |
| 76 | 160 |
| 77 | 190 |
| 78 | <100 |
| 79 | 230 |

### Sequence Listing Free Text

[SEQ ID NO: 1] primer for cloning human GSK-3β gene
[SEQ ID NO: 2] primer for cloning human GSK-3β gene
[SEQ ID NO: 3] substrate peptide derived from glycogen synthase

### INDUSTRIAL APPLICABILITY

The 2-aminopyridine of the present invention is useful as an agent for the prophylaxis or treatment of GSK-3β-related pathology or diseases.

This application is based on patent application No. 2006-278026 filed in Japan, the contents of which are incorporated in full herein by this reference.

1. A compound represented by the formula (IA): 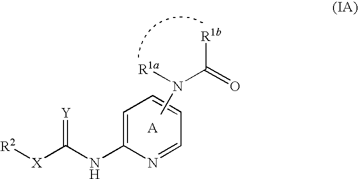
wherein
R^{1a} is a hydrogen atom, a hydrocarbon group optionally having substituent(s) or a heterocyclic group optionally having substituent(s);
R^{1b} is a hydrocarbon group optionally having substituent(s), a hydrocarbon-oxy group optionally having substituent(s) or a monocyclic heterocyclic group optionally having substituent(s); or,
R^{1a} and R^{1b} optionally form, together with the nitrogen atom and carbon atom they are bonded to, a monocyclic to tricyclic nitrogen-containing heterocycle having an oxo group and optionally having substituent(s) besides the oxo group;
R² is a hydrocarbon group optionally having substituent(s) or a heterocyclic group optionally having substituent(s);
X is an imino optionally having a substituent, —O—, —CO—NH— or a bond;
Y is an oxygen atom or a sulfur atom; and
ring A is a pyridine ring optionally further having 1 to 3 substituents selected from a halogen atom and a lower alkyl group,
or a salt thereof,
provided that tert-butyl [2-({[(9-oxo-9H-fluoren-4-yl)amino]carbonyl}amino)pyridin-4-yl]carbamate is excluded.

2. The compound of claim 1, which is a compound represented by the formula (I): 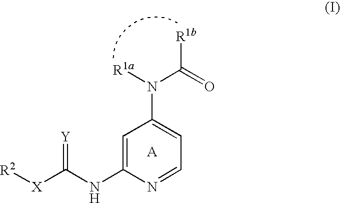
wherein each symbol is as defined in claim 1,
or a salt thereof
provided that tert-butyl [2-({[(9-oxo-9H-fluoren-4-yl)amino]carbonyl}amino)pyridin-4-yl]carbamate is excluded.

3. The compound of claim 1, wherein R^{1a} is a hydrogen atom or a hydrocarbon group optionally having substituent(s).

4. The compound of claim 1, wherein R^{1b} is a hydrocarbon group optionally having substituent(s) or a 5- or 6-membered aromatic heterocyclic group optionally having substituent(s).

5. The compound of claim 1, wherein
R^{1a} and R^{1b} optionally form, together with the nitrogen atom and carbon atom they are bonded to, a monocyclic to tricyclic nitrogen-containing heterocycle having an oxo group and optionally having substituent(s) besides the oxo group, wherein the nitrogen-containing heterocycle is
(a) a 5-membered nitrogen-containing heterocycle,
(b) a bicyclic nitrogen-containing heterocycle formed by condensation of a 5-membered nitrogen-containing heterocycle and a 6-membered aromatic ring or a C₅₋₆ cycloalkane,
(c) a bicyclic nitrogen-containing heterocycle which is a spiro ring formed by a 5-membered nitrogen-containing heterocycle and a 6-membered aromatic ring or a C₅₋₆ cycloalkane, or
(d) a tricyclic nitrogen-containing heterocycle wherein a 5-membered nitrogen-containing heterocycle and a benzene ring are condensed, and the 5-membered nitrogen-containing heterocycle and a C₅₋₆ cycloalkane form a spiro ring.

6. The compound of claim 1, wherein R² is a C₁₋₄ alkyl group substituted by 5- or 6-membered nitrogen-containing heterocyclic group(s).

7. The compound of claim 1, wherein X is an imino optionally having a substituent or a bond.

8. The compound of claim 1, wherein Y is an oxygen atom.

9. The compound of claim 1, wherein ring A is a pyridine ring without further substituent.

10. The compound of claim 1, which is
N-(4-(2-oxo-4-phenylpyrrolidin-1-yl)pyridin-2-yl)-N′-(pyridin-2-ylmethyl)urea,
N-(4-(2-oxo-5-phenyl-1,3-oxazolidin-3-yl)pyridin-2-yl)-N′-(pyridin-2-ylmethyl)urea,
1-(4-(6-methyl-2-oxo-1,3-benzoxazol-3 (2H)-yl)pyridin-2-yl)-3-(pyridin-2-ylmethyl)urea, or
N-(2-(((pyridin-2-ylmethyl)carbamoyl)amino)pyridin-4-yl)pyridine-2-carboxamide.

11. A prodrug of the compound of claim 1.

12. A pharmaceutical agent comprising the compound of claim 1 or a prodrug thereof.

13. The pharmaceutical agent of claim 12, which is a GSK-3 inhibitor.

14. The pharmaceutical agent of claim 13, wherein the GSK-3 is GSK-3β.

15. The pharmaceutical agent of claim 12, which is a neural stem cell differentiation promoter.

16. The pharmaceutical agent of claim 12, which is an agent for the prophylaxis or treatment of neurodegenerative disease or diabetes.

17. The pharmaceutical agent of claim 12, which is a hypoglycemic agent.

18. A method of inhibiting GSK-3β in a mammal, which comprises administering the compound of claim 1 or a prodrug thereof to the mammal.

19. The method of claim 18, wherein the GSK-3 is GSK-3β.

20. A method of promoting differentiation of neural stem cells in a mammal, which comprises administering the compound of claim 1 or a prodrug thereof to the mammal.

21. A method for the prophylaxis or treatment of neurodegenerative disease or diabetes in a mammal, which comprises administering the compound of claim 1 or a prodrug thereof to the mammal.

22. A method of decreasing blood glucose in a mammal, which comprises administering the compound of claim 1 or a prodrug thereof to the mammal.

23-27. (canceled)

28. A compound represented by the formula (I″): 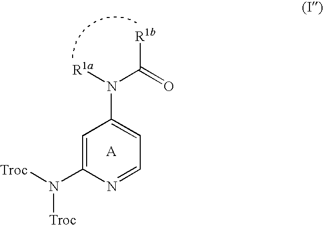
wherein
R^{1a} is a hydrogen atom or a hydrocarbon group optionally having substituent(s);
R^{1b} is a hydrocarbon group optionally having substituent(s), a hydrocarbon-oxy group optionally having substituent(s) or a 5- or 6-membered aromatic heterocyclic group optionally having substituent(s); or,
R^{1a} and R^{1b} optionally form, together with the nitrogen atom and carbon atom they are bonded to, a monocyclic to tricyclic nitrogen-containing heterocycle having an oxo group and optionally having substituent(s) besides the oxo group;
Troc is a 2,2,2-trichloroethoxycarbonyl group; and
ring A is a pyridine ring optionally further having 1 to 3 substituents selected from a halogen atom and a lower alkyl group,
or a salt thereof.


SEQUENCE LISTING (3 sequences)

SEQ ID NO: 1 - DNA, 65 bp, Artificial sequence
  Synthetic construct; primer for cloning human GSK3beta gene
aaagaattcaccatggactacaaggacgacgatgacaagtcagggcggcccagaaccacctcctt

SEQ ID NO: 2 - DNA, 39 bp, Artificial sequence
  Synthetic construct; primer for cloning human GSK3beta gene
aaaagtcgactcaggtggagttggaagctgatgcagaag

SEQ ID NO: 3 - protein, 26 aa, Artificial sequence
  Synthetic construct; substrate peptide derived from glycogen synthase
YRRAAVPPSPSLSRHSSPHQSEDEEE